(19) United States
(12) Patent Application Publication
(10) Pub. No.: US 2025/0332351 A1
(43) Pub. Date: Oct. 30, 2025

(54) PRIMARY CONTAINERS WITH IMPROVED PROTEIN DRUG STABILITY AND LOWER IMMUNE RESPONSE

(71) Applicant: SIO2 MEDICAL PRODUCTS, LLC, Auburn, AL (US)

(72) Inventors: Robert S. Abrams, New York, NY (US); Robert J. Pangborn, Harbor Springs, MI (US); Theodore W. Randolph, Niwot, CO (US); Christopher Weikart, Auburn, AL (US)

(21) Appl. No.: 19/186,968

(22) Filed: Apr. 23, 2025

Related U.S. Application Data
(63) Continuation of application No. 17/290,226, filed on Apr. 29, 2021, now abandoned.
(60) Provisional application No. 62/752,007, filed on Oct. 29, 2018.
(60) Provisional application No. 62/891,467, filed on Aug. 26, 2019.
(60) Provisional application No. 62/893,829, filed on Aug. 30, 2019.

(51) Int. Cl. A61M 5/31; A61M 5/315; A61M 5/32; C09D 183/04; C09D 189/00

(52) U.S. Cl. CPC A61M 5/3129; A61M 5/315; A61M 5/3202; C09D 183/04; C09D 189/00; A61M 2205/0238; A61M 2205/331; A61M 2205/70; A61M 2207/00

(57) ABSTRACT

A primary drug container is described having an injection-molded thermoplastic wall having an internal surface defining a lumen, a PECVD (plasma-enhanced chemical vapor deposition) drug-contact coating, and a polypeptide composition contained in the lumen. The drug-contact coating is on or adjacent to the internal surface, positioned to contact a fluid in the lumen, and consists essentially of SiOxCyHz. The primary drug container contains between a lower limit of 1,000 and an upper limit of 100,000 particles having effective spherical diameters greater than 2 and no more than 10 micrometers (μm) per mL of solution.

### PRIORITY AND INCORPORATION BY REFERENCE

This application claims the priority of each of the following: U.S. Provisional Patent Application Ser. No. 62/752,007, filed Oct. 29, 2018; U.S. Provisional Application Ser. No. 62/891,467, filed Aug. 26, 2019; and U.S. Provisional Application Ser. No. 62/893,829, filed Aug. 30, 2019. Each application identified in this paragraph is incorporated by reference in this specification in its entirety to provide continuity of disclosure.

### BACKGROUND

Biologic drugs are a class of therapeutics that are produced by means of biological processes involving recombinant DNA. Biologic drugs include therapeutic proteins. Traditionally, these drugs have been stored in primary containers composed of Type 1 borosilicate glass. These primary containers include vials, pre-filled syringes and cartridges. The drugs are stored in the primary containers through their shelf life.

It has been observed that proteins based drugs can denature. Proteins may denature by unfolding or partially unfolding. Such conformationally-perturbed species are prone to aggregate, which may result in the presence of particles in the drug product. The primary container can cause protein to denature. The following factors have been identified:
The air/liquid interface—the air headspace in the container. The air/liquid interface is a major source of protein aggregation. Typically, this interface is much larger in vials than pre-filled syringes.
The interface between the drug and container walls—solid/liquid interface
Silicone oil lubricant—Droplets of silicone oil detach from the syringe wall and interact with the drug. These droplets cause proteins in the liquid to unfold and potentially to aggregate.

A big problem with biologic drugs is the possibility that they may provoke an adverse immune response when administered to patients. An immune response can be caused by aggregates (particles) in the drug that are injected into the patient. These aggregates cause the production of antibodies in the patient that may: (1) render the drug ineffective or (2) cause a severe immune response. A small quantity of particles can cause an immune response. The proportion of proteins that have aggregated in the drug may be very, very small but can cause an immune response. For example, patients treated with biologic drugs for multiple sclerosis and Crohn's Disease patients may develop an immune response within 2 years. This may reduce the efficacy of the drug, require the patient to stop taking the drug and/or require the patient to switch drugs.

The number of protein-based drugs has increased significantly over the past five years and this trend will continue. Drug therapies are being used to treat more chronic indications. This means that patients are taking the drugs longer and are more prone to side effects caused by the drug. Previously, protein drugs were taken for acute indications and side effects were limited.

The amount of particulate contaminants in the drug may increase over the shelf life. Millions of particles per mL may be detected in formulations of protein therapeutics. This number of concentration typically represents a very small mass of particles, but this may be enough to cause an immune response.

A primary drug container is a container with which the drug makes direct contact during storage. Several non-limiting examples of primary drug containers are prefilled syringes, cartridges, and vials.

The problems of particle contamination in primary drug containers are particularly acute in such containers made of glass. See for example Pharmacopeia (USP), Chapter 1660, Evaluation of The Inner Surface Durability of Glass Containers, which identifies inhomogeneity in the glass surface, caused by phase separation and other factors, which can lead to breakdown of the glass, producing small particles in a drug contacting the glass.

### SUMMARY

One aspect of the present invention is a primary drug container comprising an injection-molded thermoplastic wall having an internal surface defining a lumen, a PECVD (plasma-enhanced chemical vapor deposition) drug-contact coating, and a polypeptide composition contained in the lumen. The drug-contact coating is on or adjacent to the internal surface, positioned to contact a fluid in the lumen, and consists essentially of SiOₓC_{y}H_{z}. In SiOₓC_{y}H_{z}, x is between 0.5 and 2.4, optionally between 1.3 and 1.9, as measured by x-ray photoelectron spectroscopy (XPS), y is between 0.6 and 3, optionally between 0.8 and 1.4, as measured by XPS; and z is between 2 and 9, optionally between 2 and 6, as measured by Rutherford backscattering. The primary drug container contains between a lower limit of 1,000 and an upper limit of 100,000 particles having effective spherical diameters greater than 2 and no more than 10 micrometers (μm) per mL of solution.

Another aspect of the invention is a primary drug container including a wall and a PECVD drug-contact coating. The wall has an internal surface defining a lumen. The PECVD drug-contact coating is supported on or adjacent to the internal surface and positioned to contact a fluid in the lumen. The primary drug container contains less than 10000 particles having diameters between 2 and 50 micrometers (μm) per mL of solution, measured by light obscuration particle count testing.

Other aspects of the invention will be apparent from the specification, drawings, and claims of this specification.

### BRIEF DESCRIPTION OF DRAWING FIGURES

FIG. 1 is a plot showing the number of less than 10 μm (micrometer) diameter particles per mL in lots of uncoated 6 mL vials using the particle count protocol stated in Example 1 later in this specification.

FIG. 2 is a plot showing the number of less than 25 μm (micrometer) diameter particles per mL in lots of uncoated 6 mL vials using the particle count protocol stated in Example 1 later in this specification.

FIG. 3 is a plot showing the number of less than 50 μm (micrometer) diameter particles per mL in lots of uncoated 6 mL vials using the particle count protocol stated in Example 1 later in this specification.

FIG. 4 is a plot showing the number of 50 to 100 μm (micrometer) diameter particles per 100 containers in lots of uncoated 6 mL vials using the particle count protocol stated in Example 1 later in this specification.

FIG. 5 is a plot showing the number of greater than 100 μm (micrometer) diameter particles per 100 containers in lots of uncoated 6 mL vials using the particle count protocol stated in Example 1 later in this specification.

FIG. 6 is a diagrammatic sectional view, with enlarged portion, of a staked-needle cyclic olefin polymer syringe barrel or wall, showing a trilayer coating (adhesion, barrier, and pH protective), with the pH protective coating on the drug-contact surface, and optionally a further coating of a silicon oil free lubricant (PECVD lubricious coating) instead defining the drug contact surface.

FIG. 7 is a comparison of the number of particles per mL resulting from the trilayer and optional further coating of FIG. 6, on the left, with the number of particles per mL on a glass syringe conventionally lubricated with a surface deposit of silicone oil, on the right, showing more than 10 times as many particles of 2-50 μm diameter, using the particle count protocol stated in Example 1 later in this specification.

FIG. 8 is an output field of the Beckman Coulter HIAC 9703+ Liquid Particle Counter using the corresponding particle count protocol stated later in this specification for the glass syringe conventionally lubricated with a surface deposit of silicone oil (polydimethylsiloxane) of FIG. 7, identifying numerous fluid lubricant particles.

FIG. 9 is a plot superimposing the data of FIGS. 21 (C3a data, lower plot) and 22 (C5a data, upper plot) for particles having diameters from 2 to 10 microns for all particulate samples generated by various stresses/formulations/container types.

FIG. 10 is a plot similar to FIG. 9 showing the corresponding data for particles having diameters greater than 10 microns. No correlation is demonstrated for complement activation vs. particle concentration for particles greater than 10 micron diameter.

FIG. 11 is a plot similar to FIG. 9 showing that complement activation in response to IVIg particles in vials is particle-mediated for particles having diameters from 2 to 10 microns.

FIG. 12 is a plot similar to FIG. 11 showing that complement activation in response to IVIg particles in syringes is particle-mediated for particles having diameters from 2 to 10 microns.

FIG. 13 is a collage resulting from flow imaging microscopy of stressed IVIg samples processed as described in Example 4. The collage is a randomly-selected set of images of particles present in IVIg samples that had been processed by overnight shaking in glass syringes on an orbital shaker.

FIG. 14 is a collage similar to FIG. 13 resulting from flow imaging microscopy of stressed IVIg samples. The collage is a randomly-selected set of images of particles present in IVIg samples that had been processed by 10 days of end-over-end rotation in SiOPlas™ syringes.

FIG. 15 is a collage similar to FIG. 13 resulting from flow imaging microscopy of stressed IVIg samples. The collage is a randomly-selected set of images of particles present in IVIg samples that had been processed by overnight shaking in SiOPlas™ syringes on an orbital shaker.

FIG. 16 is a collage similar to FIG. 13 resulting from flow imaging microscopy of stressed IVIg samples. The collage is a randomly-selected set of images of particles present in IVIg samples that had been processed by freeze-thawing six times in glass vials.

FIG. 17 is a collage similar to FIG. 13 resulting from flow imaging microscopy of stressed IVIg samples. The collage is a randomly-selected set of images of particles present in IVIg samples that had been processed by 10 days of end-over-end rotation in silicone oil-lubricated glass syringes.

FIG. 18 is a collage similar to FIG. 13 resulting from flow imaging microscopy of stressed IVIg samples. The collage is a randomly-selected set of images of particles present in IVIg samples that had been processed by freeze-thawing six times in SiOPlas™ vials.

FIG. 19 is a plot of Example 4 data showing that protein particles within IVIg samples did not stimulate release of C4a concentration in human serum samples. Concentrations are reported as fold increases vs. C4a levels for saline control samples. Particle concentrations refer to concentrations in IVIg formulations prior to 10-fold dilution into human serum. Line represents a least-squares linear fit, with correlation coefficient r²=0.005.

FIG. 20 is a plot of Example 4 data showing the effect of sample particle levels on Bb concentration in human serum samples. Concentrations are reported as fold increases vs. Bb levels for saline control samples. Particle concentrations refer to concentrations in IVIg formulations prior to 10-fold dilution into human serum. Line represents a least-squares linear fit, with correlation coefficient r²=0.94.

FIG. 21 is a plot of Example 4 data showing the effect of sample particle levels on C3a concentration in human serum samples. Concentrations are reported as fold increases vs. C3a levels for saline control samples. Particle concentrations refer to concentrations in IVIg formulations prior to 10-fold dilution into human serum. Line represents a least-squares linear fit, with correlation coefficient r²=0.85.

FIG. 22 is a plot of Example 4 data showing the effect of sample particle levels on C5a concentration in human serum samples. Concentrations are reported as fold increases vs. C5a levels for saline control samples. Particle concentrations refer to concentrations in IVIg formulations prior to 10-fold dilution into human serum. Line represents a least-squares linear fit, with correlation coefficient r²=0.99.

### DETAILED DESCRIPTION

### I-A Drug

Optionally in any embodiment, the primary drug container contains a polypeptide composition, for example a biopharmaceutical composition, in the lumen in contact with the PECVD coating.

Optionally in any embodiment, the primary drug container contains a protein composition in the lumen in contact with the PECVD coating.

Optionally in any embodiment, the primary drug container contains a biopharmaceutical drug from the following list of drugs and their indications, or any combination of two or more of these, contained in the lumen in contact with the PECVD coating.

### List of Biological Drugs:

1. TNF mAb, rDNA (Inflectra—infliximab-dyyb)) granted on Apr. 5, 2016 to Janssen/Johnson & Johnson as a biosimilar of Remicade, including extrapolation of all of Remicade's current approved indications: manufactured by Celltrion.
2. Polydeoxyribonucleotides, porcine-derived (Defitelio—defibrotide sodium; polydeoxyribonucleotide, sodium salt) granted on Mar. 30, 2016 to Jazz Pharmaceuticals for treatment of hepatic veno-occlusive disease (VOD) with additional kidney or lung abnormalities after receiving a stem cell transplant from blood or bone marrow called hematopoietic stem cell transplantation (HSCT).
3. IL-17 Mab, rDNA (Taltz; ixekizumab) granted on Mar. 22, 2016 to Eli Lilly & Co. for treatment of adults with moderate-to-severe plaque psoriasis.
4. Anthrax Mab, rDNA (Anthim; Bacillus anthracis (anthrax) Protective Antigen (PA) monoclonal antibody, recombinant; ETI-204) granted on Mar. 22, 2016 to Elusys Therapeutics, Inc. for prophylaxis against inhalational anthrax infection (for the U.S. biodefense Strategic National Stockpile (SNS)).
5. Factor VIII. rDNA (Kovaltry; Antihemophilic Factor (Recombinant)) granted on Mar. 17, 2016 to Bayer AG for treatment of hemophilia (with less frequent injections required).
6. Insulin glargine. rDNA (Basaglar; Abasria; LY2963016) granted on Dec. 16, 2015 to Boehringer Ingelheim for treatment of diabetes; a 505(b)(2) drug approval, which many would now call a biosimilar.
7. Factor X, blood-derived (Coagadex; Coagulation Factor X (Human)) granted on Dec. 16, 2015 to Bio Products Laboratory Ltd. for treatment of for hereditary Factor X deficiency.
8. von Willebrand factor, rDNA (Vonvendi) granted on Dec. 9, 2015 to Baxalta U.S. (formerly Baxter) for treatment of von Willebrand's disease (VWD).
9. Lyosomal acid lipase, chicken egg-expressed (Kanuma; sebelipase alfa) granted on Dec. 8, 2015 to Alexion (from acquisition of Synageva) for treatment of lyosomal acid lipase (LAL) deficiency (2nd product from genetically engineered animals).
10. Influenza vaccine, quadravelent (Fluad; influenza vaccine, inactivated, egg-cultured, Quadravalent with MF59/squalene adjuvant) granted on Dec. 3, 2015 to Novartis (with product and its approval to be transferred to Seqirus, part of CSL Group) for prevention of influenza; first U.S. non-aluminum-based adjuvanted influenza vaccine.
11. SLAMF7 mAb, rDNA (Empliciti; elotuzumab; Signaling Lymphocyte Activation Molecule Family member 7 monoclonal antibody, recombinant) granted on Nov. 30, 2015 to Bristol Myers Squibb (BMS; with Abbvie) for use in combination with lenalidomide and dexamethasone for the treatment of patients with multiple myeloma who have received 1-3 prior therapies.
12. EGFr mAb, rDNA (Portrazza—necitumumab; epidermal growth factor receptor monoclonal antibody) granted on Nov. 24, 2015 to Janssen Biotech for treatment of metastatic squamous non-small cell lung cancer(VWD) in combination with gemcitabine and cisplatin.
13. CD38 mAb, rDNA Darzalex—daratumumab) granted on Nov. 16, 2015 to Eli Lilly for treatment of multiple myeloma.
14. Factor VIII, rDNA, pegylated (Adynovate—BAX 111) granted on Nov. 11, 2015 to Baxalta (formerly Baxter) for treatment of hemophilia A.
15. IL-5 mAb, rDNA (Nucala; mepolizumab) granted on Nov. 4, 2015 to GlaxoSmithKline (GSK) for treatment of asthma.
16. HSV-1/GM-CSF, rDNA, rDNA (Imlygic—alimogene laherparepvec; a live herpes simplex virus type 1 (HSV-1) oncolytic virus resulting in expression of GM-CSF) granted on Oct. 27, 2015 to Alexion Pharmaceuticals for treatment of unresectable recurrent cutaneous melanoma.
17. Alkaline phosphatase, rDNA (Strensiq; asfotase alfa; an alkaline phosphatase catalytic domain fusion protein) granted on Oct. 23, 2015 to Alexion Pharmaceuticals for treatment of perinatal, infantile and juvenile-onset hypophosphatasia (HPP).
18. Dabigatran mAb, rDNA (Praxbind—idarucizumab) granted on Oct. 16, 2015 to Boehringer Ingelheim to reverse the blood-thinning effects of Pradaxa (dabigatran).
19. insulin degludec, rDNA (Tresiba—insulin degludec) granted on Oct. 16, 2015 to Novo Nordisk for treatment of diabetes mellitus.
20. Insulin degludec/aspart, rDNA (Ryzodeg 70/30—a 70/30 mixture of insulin degludec (approved the same day) and insulin aspart) granted on Oct. 16, 2015 to Novo Nordisk for treatment of diabetes mellitus.
21. Factor VIII, rDNA (Nuwiq) granted on Oct. 16, 2015 to Octapharma USA for treatment of hemophilia A.
22. PCSK9 mAb, rDNA (Repatha—evolocumab; proprotein convertase subtilisin kexin type 9 monoclonal antibody) approval granted on Aug. 27, 2015 to Amgen Inc. for treatment of high low-density lipoprotein (LDL) cholesterol levels.
23. PCSK9 mAb, rDNA (Praluent—alirocumab; proprotein convertase subtilisin kexin type 9 monoclonal antibody) approval granted on Jul. 24, 2015 to Sanofi and Regeneron Pharmaceuticals for treatment of high low-density lipoprotein (LDL) cholesterol levels.
24. Crotalidae Immune F(ab′)2 (Equine) (Anavip) approval granted on May 6, 2015 to ProFibrix, BV with manufacture by Instituto Bioclon S.A. (Mexico) for treatment of North American rattlesnake bites.
25. Fibrin Sealant (RAPLIXA; RaplixaSpray—human plasma-derived fibrinogen and thrombin) approval granted on Apr. 30, 2015 to The Medicines Company (originally developed by ProFibrix, BV, which was acquired) for treatment of mild to moderate bleeding in adults undergoing surgery when control of bleeding by standard surgical techniques is ineffective or impractical.
26. Factor IX, rDNA (Coagulation Factor IX (Recombinant)—Ixinity) approval granted on Apr. 29, 2015 to Cangene/Emergent Biosolutions for treatment of hemophilia B.
27. DTaP-IPV vaccine (Quadracel—Diphtheria and Tetanus Toxoids and Acellular Pertussis Absorbed and Inactivated Poliovirus) approval granted on Mar. 26, 2015 to Sanofi Pasteur for active immunization against diphtheria, tetanus, pertussis and poliomyelitis in children 4 through 6 years of age.
28. Anthrax immune globulin (Anthrax Immune Globulin Intravenous (Human)—Anthracil; AIGIV) approval granted on Mar. 24, 2015 to Emergent BioSolutions Inc. for treatment of inhalational anthrax in combination with appropriate antibacterial drugs.
29. GD2 mAb, rDNA (dinutuximab—Unituxin; ch14.18) approval granted on Mar. 10, 2015 to United Therapeutics Corp. (in combination with granulocyte-macrophage colony-stimulating factor (GM-CSF), interleukin-2 (IL-2), and 13-cis-retinoic acid (RA)) for the treatment of pediatric patients with high-risk neuroblastoma.
30. G-CSF, rDNA/Sandoz (filgrastim-sndz—Zarxio; Zarzio; Granulocyte Colony Stimulating Factor, recombinant) approval granted on Mar. 6, 2015 to Sandoz/Novartis; first biosimilar approval; for treatment of neutropenia (same indications as Neupogen).
31. PD-1 mAb, rDNA/Sandoz (nivolumab—Opdivo; ONO-4538; BMS-936558; MDX1106; programmed cell death 1 monoclonal antibody, recombinant) approval granted on Mar. 4, 2015 to Bristol-Myers Squibb Co. (BMS; licensed from Ono Pharm.) for treatment of patients with metastatic squamous non-small cell lung cancer (NSCLC) with progression on or after platinum-based chemotherapy.
32. Insulin glargine, rDNA (Toujeo—Gly(A21)-human Insulin Arg(B31)-Arg(B32)-OH, recombinant) approval granted on Feb. 25, 2015 to Sanofi for once-daily long-acting basal insulin treatment in adults living with type 1 and type 2 diabetes.
33. Parathyroid hormone (1-84), rDNA (Natpara; Preos; Preotact) approval granted on Jan. 25, 2015 to NPS Pharmaceuticals, being acquired by Shire, for treatment of hypocalcemia (low blood calcium levels) in patients with hypoparathyroidism.
34. Neisseria meningitidis vaccine (secukinumab—Bexsero; Cosentyx) approval granted on Jan. 23, 2015 to Novartis for prevention of invasive meningococcal disease.
35. IL-17 mAb, rDNA (secukinumab—Cosentyx) approval granted on Jan. 21, 2015 to Novartis for treatment of adults with moderate to severe plaque psoriasis.
36. Programmed death receptor-1 mAb, rDNA (nivolumab—Opdivo) approval granted on Dec. 22, 2014 to Bristol-Myers Squibb for treatment of advanced melanoma (for patients with unresectable or metastatic melanoma and disease progression after treatment with Yervoy, in patients with BRAF V600 mutation positive tumors).
37. Influenza Vaccine, 4-valent, i.d. (Fluzone Intradermal Quadrivalent) approval granted on Dec. 12, 2014 to Sanofi Pasteur for prophylactic use in adults age 18-64.
38. HPV vaccine, 9-valent, rDNA (Human Papillomavirus 9-valent Vaccine, Recombinant—Gardasil 9) approval granted on Dec. 10, 2014 to Merck & Co. for prophylactic use in females age 9-26.
39. CD3-CD19 bi-specific mAb, rDNA (blinatumomab—AMG103; CD19-CD3 bispecific monoclonal antibody; CD3-CD19bi-specific T-cell engager (BiTE)) accelerated approval granted on Dec. 3, 2014 to Amgen for treatment of Philadelphia chromosome-negative relapsed/refractory B-precursor acute lymphoblastic leukemia (ALL); breakthrough therapy designation.
40. Meningococcal B vaccine (meningococcal group B vaccine—Trumemba) accelerated approval granted on Oct. 29, 2014 to Pfizer for active immunization to prevent invasive Neisseria meningitidis serogroup B in those 10-25 years of age.
41. Factor VIII, porcine rDNA (Antihemophilic Factor (Recombinant), Porcine (pig) Sequence—Obizur; OBI-1; Factor VIII, porcine recombinant) approval granted on Oct. 24, 2014 to Baxter for treatment of adult patients with adults with acquired (not-hereditary) hemophilia A.
42. Glucagon-like peptide-1, rDNA (Trulicity) approval granted on Sep. 18, 2014 to Eli Lilly & Co. for treatment of adult patients with adults with type 2 diabetes.
43. Immune Globulin & Hyaluronidase rDNA (Immune Globulin Infusion 10% (Human) with Recombinant Human Hyaluronidase—HYQVIA; Gammagard combined with Hylenex) approval granted on Sep. 12, 2014 to Baxter (and Halozyme Therapeutics) for treatment of adult patients with primary immunodeficiency (PI).
44. PD-1 mAb, rDNA (Keytruda—pembrolizumab; MK-3475) approval granted on Sep. 4, 2014 to Merck & Co. for treatment of patients with advanced melanoma not responding to other therapies.
45. Insulin glargine, rDNA/Lilly (Basaglar) tentative approval granted on Aug. 18, 2014 to Eli Lilly & Co., partnered with Boehringer Ingelheim, for the treatment of diabetes.
46. Interferon beta-1a, PEG-, rDNA (Plegridy) granted on Aug. 15, 2014 to Biogen Idec for the treatment of relapsing forms of multiple sclerosis (RMS).
47. C1-esterase inhibitor, rDNA (conestat alfa—Rhucin; Ruconest; C1INH; C1-INH; human complement C1 esterase inhibitor, recombinant, transgenic rabbits) granted on Jul. 17, 2014 to Salix Pharmaceuticals, Ltd. (and Pharming Group NV) for the treatment of acute angioedema attacks in adult and adolescent patients with hereditary angioedema (HAE).
48. Insulin, rDNA, inhaled/MannKind (insulin human (rDNA origin)) Inhalation Powder—Afrezza; Afresa Inhalation Powder; Technosphere insulin) granted on Jun. 27, 2014 to MannKind Corp, to improve glycemic control in adults with diabetes mellitus.
49. Factor VIII/Biogen-Idec, rDNA (Eloctate) granted on Jun. 6, 2014 to Biogen Idec Inc. for treatment of hemophilia A.
50. Integrin mAb, rDNA (Entyvio—vedolizumab) granted on May 20, 2014 to Takeda Pharmaceuticals America, Inc. for treatment of ulcerative colitis and Crohn's disease.
51. IL-6 mAb, rDNA (Sylvant—siltuximab; CNTO 328) granted on Apr. 23, 2014 to Janssen/J&J for treatment of multicentric Castleman's disease (MCD).
52. VEGF-2 mAb, rDNA (VEGRFr mAb—Cyramza; ramucirumab) granted on Apr. 21, 2014 to Eli Lilly & Co. for treatment of advanced gastic cancers.
53. GLP-1/Albumin fusion protein, rDNA (Tanzeum—glucagon-like peptide-1 (GLP-1)-albumin fusion protein) granted on Apr. 15, 2014 to GlaxoSmithKline (GSK) for glycemic control in type 2 diabetes.
54. Factor IX-Fc fusion protein, rDNA (Coagulation Factor IX (Recombinant), Fc Fusion Protein—Alprolix; Factor IX-XTEN) granted on Mar. 28, 2014 to Biogen Idec for treatment of hemophilia B.
55. Hyaluronic acid, cross-linked (Monovisc) granted on Feb. 25, 2014 to Anika Therapeutics for osteoarthritis treatment of the knee.
56. Leptin, rDNA (Metreleptin—methionyl human leptin, recombinant) granted on Feb. 24, 2014 as replacement therapy to treat the complications of leptin deficiency.
57. N-acetylgalactosamine-6-sulfatase, rDNA (elosulfase alfa—Vimizim; N-acetylgalactosamine-6-sulfatase; rhGALNS; BMN-110, elosulfase alfa; chondroitin sulfatase) granted on Feb. 14, 2014 to BioMarin (marketed by DePuy Synthes, a unit of Johnson & Johnson) for treatment of mucopolysaccharidosis type IV A (Morquio A syndrome).
58. Factor XIII, rDNA (Coagulation Factor XIII A-Subunit (Recombinant)—Tretten) granted on Dec. 23, 2013 to Novo Nordisk A/S for routine prevention of bleeding in adults and children with congenital Factor XIII A-subunit deficiency (hemophilia).
59. Influenza vaccine, H5N1 (Influenza A (H5N1) Virus Monovalent Vaccine, Adjuvanted) granted on Nov. 22, 2013 to ID Biomedical/GSK for prevention of H5N1 influenza, commonly known as avian or bird flu; full BLA, but only intended for pandemic/biodefense stockpile use, granted to this egg-cultured AS03-adjuvanted vaccine.
60. CD20 mAb, rDNA/Roche (obinutuzumab—Gazyva; GA101) granted on Nov. 1, 2013 to Genentech/Roche for use in combination with chlorambucil chemotherapy for the treatment of previously untreated chronic lymphocytic leukemia (CLL).
61. Tetanus and Diphtheria Toxoids Adsorbed (Tenivac) granted on Oct. 25, 2013 to Sanofi for tetanus and diphtheria prevention.
62. Factor VIII, rDNA/Novo (Antihemophilic Factor (Recombinant)—NovoEight; Factor VIII, recombinant) granted on Oct. 15, 2013 to Novo Nordisk for treatment of hemophilia A.
63. Influenza vaccine, quadrivalent/GSK (Flulaval Quadrivalent) granted on Aug. 16, 2013 to GlaxoSmithKline (GSK) for influenza prophylaxis.
64. TNF Mab, rDNA, human/J&J (golimumab—Simponi Aria) full BLA granted on Jul. 18, 2013 to Janssen Biotech, Johnson & Johnson for treatment of moderately to severely active rheumatoid arthritis.
65. Factor IX, rDNA/Baxter (Coagulation Factor IX (Recombinant)—Rixubis) granted on Jun. 27, 2013 to Baxter Healthcare for treatment of hemophilia B.
66. Influenza vaccine, quadrivalent/Sanofi (Fluzone Quadrivalent) granted on Jun. 10, 2013 to Sanofi for influenza prophylaxis.
67. Prothrombin Complex/CSL (Prothrombin Complex Concentrate (Human)—Kcentra) granted on Apr. 29, 2013 to CSL Behring GmbH for urgent reversal of acquired coagulation factor deficiency induced by vitamin K antagonist (VKA, e.g., warfarin) therapy in adult patients with acute major bleeding.
68. Botulism Antitoxin/A-G (Botulism Antitoxin Heptavalent (A, B, C, D, E, F, G)-(Equine); Clostridium botulinum toxin immune globulin, equine) granted on Mar. 23, 2013 to Cangene Corp. for treatment of botulism following documented or suspected exposure to botulinum neurotoxin.
69. HER2 receptor Mab-DM1, rDNA (ado-trastuzumab emtansine—Kadcyla; trastuzumab emtansine; trastuzumab-DM1; T-DM1; trastuzumab-MCC-DM1; herceptin-DM1 conjugate) granted on Feb. 22, 2013 to Genentech/Roche for treatment of HER2-positive metastatic breast cancer (mBC).
70. apolipoprotein B, antisense (mipomersen—Kynamro; ISIS 301012) granted on Jan. 17, 2013 to Isis Pharmaceuticals and Genzyme/Sanofi for use as an adjunct to lipid-lowering drugs and diet to reduce low-density lipoprotein-cholesterol (LDL-C), apolipoprotein B (Apo B), total cholesterol (TC), and non-high-density lipoprotein-cholesterol (non HDL-C) in patients with homozygous familial hypercholesterolemia (HoFH).
71. Plasma SD/Octapharma (Octaplas—Plasma, solvent-detergent inactivated) granted on Jan. 17, 2013 to Octapharma AG for needed replacement of clotting proteins (coagulation factors).
72. Influenza vaccine, rHA, rDNA (Influenza Vaccine, Purified Recombinant Influenza Hemagglutinin—FluBlok; influenza hemagglutinin vaccine, insect cell-cultured, recombinant) granted on Jan. 17, 2013 to Protein Sciences Corp., with marketing by Emergent Biosolutions, Inc., for prevention of influenza in persons 18-49 years old.
73. Glucagon-like peptide 2, rDNA (teduglutide (rDNA origin)—GATTEX) granted on Dec. 21, 2012 to NPS Pharmaceutical for treatment of adults with short bowel syndrome (SBS) who need additional nutrition from intravenous feeding (parenteral nutrition).
74. Immune Globulin (IGIV)/Biotest (Immune Globulin Intravenous (Human)—Bivigam) granted on Dec. 20, 2012 to Biotest Pharmaceuticals Corp. for treatment of primary immune deficiency disorders (PIDD).
75. Varicella-Zoster Immune Globulin/Cangene (Varicella-Zoster Immune Globulin (Human)—VariZIG; VZVIG) granted on Dec. 19, 2012 to Cangene Corp. for post-exposure prophylaxis of varicella in high risk individuals to reduce the severity of varicella.
76. Fluarix Quadravalent (Influenza Virus Vaccine—Fluarix Quadravalent) granted on Dec. 17, 2012 to GlaxoSmithKline (GSK), for prevention of disease caused by the four seasonal influenza (flu) virus subtypes A and type B strains represented by antigens in the vaccine.
77. Anthrax Mab, rDNA/HGSI (raxibacumab—ABthrax; Bacillus anthracis protective antigen human monoclonal antibody, recombinant) granted on Dec. 14, 2012 to Human Genome Sciences Inc., a subsidiary of GlaxoSmithKline (GSK), for treatment of inhalational anthrax and prevention of inhalational anthrax when alternative therapies are not available or not appropriate.
78. Fibrin Sealant Patch/J&J (Fibrin Sealant Patch-Human Fibrinogen and Human Thrombin—EVARREST Fibrin Sealant Patch) granted on Dec. 7, 2012 to Ethicon Biosurgery, Johnson & Johnson (J&J), with manufacture by Omrix Biopharmaceuticals Ltd. (Israel) for use as an aid in stopping problematic bleeding during surgery.
79. Influenza vaccine, MDCK cultured/Novartis (Flucelvax; Optaflu; Influenza virus vaccine, inactivated) granted on Nov. 20, 2012 to Novartis for prevention of seasonal influenza in people ages 18 years and older (the first cell cultured influenza vaccine in the U.S.).
80. Microplasmin, rDNA (Ocriplasmin—Jetrea) granted on Oct. 18, 2012 to ThromboGenics for treatment of symptomatic vitreomacular adhesion.
81. G-CSF, rDNA/Teva (filgrastim; same active agent as Neupogen and TevaGrastin, approved as a biosimilar of Neupogen in the EU) Full BLA granted on Aug. 30, 2012 to Sicor Biotech (Teva Pharmaceuticals) to reduce the duration of severe neutropenia in patients with certain types of cancer (non-myeloid malignancies) receiving chemotherapy that affects the bone marrow.
82. VEGF Trap, rDNA (ziv-aflibercept—Zaltrap) granted on Aug. 3, 2012 to Sanofi (with Regeneron) for use in combination with 5-flourouracil, leucovorin, irinotecan-(FOLFIRI) for treatment of metastatic colorectal cancer (mCRC) resistant to or having progressed following an oxaliplatin-containing regimen.
83. MenC-Hib vaccine (Meningococcal Groups C and Y and Haemophilus b Tetanus Toxoid Conjugate Vaccine—MenHibrix) granted on Jun. 14, 2012 to GlaxoSmithKline for prevention of invasive disease caused by Neisseria meningitidis serogroups C and Y and Haemophilus influenzae type b (a combination of prior-approved vaccines).
84. HER2 receptor Mab, rDNA/2C4 (pertuzumab—Perjeta; Omnitarg; 2C4) granted on Jun. 8, 2012 to Genentech/Roche for use combination with Herceptin (trastuzumab) and docetaxel chemotherapy for the first-line treatment of HER2-positive metastatic breast cancer.
85. Glucocerebrosidase, rDNA/Protalix (taliglucerase alfa—Elelyso; Uplyso; beta-glucocerebrosidase, recombinant (carrot expressed); prGCD) granted on May 1, 2012 to Protalix BioTherapeutics Inc. and Pfizer (the BLA holder) for treatment of Gaucher disease (Could be considered a biobetter version of Cerezyme from Genzyme/Sanofi).
86. Human cells, autologous/bovine collagen substrate (Allogeneic Cultured Keratinocytes and Fibroblasts in Bovine Collagen—GINTUIT) granted on Mar. 9, 2012 to Organogenesis Inc. for topical application to a surgically created vascular wound bed in the treatment of mucogingival (gums; oral tissue) conditions (first cell-based product for an oral tissue application).
87. Pancreatic enzymes (pancrelipase—Ultresa; Viokase) granted on Mar. 1, 2012 to Aptalis Pharma for treatment of orphan pancreatic insufficiency indications (First full approval for a long-marketed, grandfathered drug product).
88. Influenza vaccine, live intranasal quadravalent (FluMist Quadrivalent; Influenza Vaccine Live, Intranasal) sBLA granted on Feb. 29, 2012 to MedImmune (AstraZeneca) for prevention of seasonal influenza.
89. Carboxypeptidase, rDNA—(Glucarpidase—Voraxaze; CPG2; carboxypeptidase G2, recombinant) granted on Jan. 18, 2012 to BTG plc (formerly Protherics) for treatment of methotrexate toxicity.
90. VEGF Trap, rDNA—(Aflibercept—Eylca; VEGF Trap-Eye) granted on Nov. 18, 2011 to Regeneron Pharmaceuticals (with international marketing by Bayer) for treatment of wet (neovascular) age-related macular degeneration (AMD)
91. Asparaginase/Erwinia—(Erwinaze—asparaginase Erwinia chrysanthemi; Erwinase; L-asparagine aminohydrolase; L-asparaginase) granted on Nov. 18, 2011 to EUSA Pharma Inc. for treatment of acute lymphoblastic leukemia (ALL).
92. cord blood stem cells—(HEMACORD; Hematopoietic progenitor cells-cord (HPC-C) cell therapy) approval granted on Nov. 10, 2011 to the New York Blood Center, Inc. for use in hematopoietic stem cell transplantation procedures in patients with disorders affecting the hematopoietic (blood forming) system.
93. CD30 mAb-monomethyl auristatin E—(Adcetris; brentuximab vedotin; CD30 mAb-cytotoxin conjugate) accelerated approval with orphan status granted on Aug. 19, 2011 to Seattle Genetics, Inc. for treatment of Hodgkin lymphoma; currently the only immunotoxin in the U.S. market.
94. Centruroides (Scorpion) Immune F(ab)2 (Equine) Injection (Anascorp) granted on Aug. 3, 2011 to Rare Disease Therapeutics Inc. for treatment of scorpion stings, with manufacture by Instituto Bioclon, S.A. (Mexico).
95. Fibroblasts, autologous (azfibrocel-T—laViv; Isolagen Therapy) granted on Jun. 22, 2011 to Fibrocell Science, Inc. for treatment of the appearance of nasolabial fold wrinkles (smile lines).
96. CTLA4-Ig, rDNA (belatacept—Nulojix; BMS-224818; CTLA4-Ig mutant; cytotoxic T-lymphocyte-associated antigen 4 (CTLA-4)—Immunoglobulin G1 fragment fusion protein, recombinant) granted on Jun. 15, 2011 to Bristol-Myers Squibb (BMS) for prevention of acute rejection in adult kidney transplant patients.
97. Albumin, human—(Kedbumin) granted on Jun. 3, 2011 to Kedrion, S.p.A. for treatment of hypovolemic shock, hypoalbuminemia, prevention of central volume depletion after paracentesis due to cirrhotic ascites, Ovarian Hyperstimulation Syndrome (OHSS), Adult Respiratory Distress Syndrome (ARDS), burns, hemodialysis patients undergoing long term dialysis, patients who cannot tolerate substantial volumes of salt solution and as a priming solution for cardiopulmonary bypass.
98. CTLA-4 Mab, rDNA/Medarex (Yervoy; Ipilimumab; MDX-010; cytotoxic T lymphocyte-associated antigen-4 (CTLA-4) monoclonal antibody, recombinant) granted on Mar. 25, 2011 to Bristol-Myers Squibb (BMS) for treatment of late-stage melanoma.
99. Adenovirus Type 4 and Type 7 Vaccine, Live, Oral (composed of Adenovirus Vaccine, type 4 and Adenovirus Vaccine, type 7) granted on Mar. 16, 2011 to Teva Pharmaceuticals for immunization of U.S. military personnel only
100. B-cell-activating factor Mab, rDNA: (belimumab—Benlysta; LymphoStat-B) granted on Mar. 9, 2011 to Human Genome Sciences, Inc. (for marketing along with GlaxoSmithKline/GSK) for treatment of treatment of adults with active, autoantibody-positive systemic lupus erythematosus.
101. Factor XIII, human: (Cortifact) granted on Feb. 17, 2011 to CSL Behring for treatment of Factor XIII deficiency.
102. Urate oxidase, rDNA, PEG−: (Pegloticase—Krystexxa; Puricase; PEG-uricase; pig-baboon uric acid oxidase, recombinant, pegylated) granted on Sep. 14, 2010 to Savient Pharmaceuticals for treatment of chronic refractory gout.
103. Botulinum Toxin A/Merz: (Xeomin; Clostridium botulinum toxin type A; NT 201) granted on Jul. 30, 2010 to Merz Pharmaceuticals for treatment of adults with cervical dystonia or blepharospasm.
104. Antitrypsin, alpha-1/Kamada: (alpha-1-Proteinase Inhibitor (Human)—Glassia; Respira; alpha-1 antitrypsin; AAT; A1P1) granted on Jul. 10, 2001 to Kamada Ltd. for treatment of alpha1-antitrypsin deficiency.
105. RANKL Mab, rDNA: (Denosumab—Prolia; AMG 531; AMG 162. receptor activator of nuclear factor kappa B ligand (RANKL) monoclonal antibody, recombinant) granted on Jun. 1, 2010 to Amgen Inc. for treatment of postmenopausal women with osteoporosis at high risk for fracture
106. Glucosidase, rDNA/Lumizyme: (Alglucosidase alfa—Lumizyme; alpha glucosidase; glucosidase alpha; rhGAA)) granted on May 25, 2010 to Genzyme Corp. for treatment of Pompe disease.
107. Prostate Cancer Cellular Vaccine (rDNA): (Sipulcuccl-T—Provenge—prostatic acid phosphatase (PAP)—granulocyte macrophage-colony stimulating factor (GM-CSF) recombinant fusion protein (PAP-GM-CSF; PA2024)-sensitized autologous antigen-presenting cells (APCs); PA2024-loaded APCs; APC8015) granted on Apr. 29, 2010 to Dendreon Corp. for treatment of asymptomatic or minimally symptomatic metastatic prostate cancer resistant to standard hormone treatment.
108. Pancreatic Enzymes/J&J: (Pancreaze; Pancreatic Enzyme Product) NDA granted on Apr. 12, 2010 to Johnson & Johnson (J&J) for treatment of pancreatic insufficiency.
109. Fibrin Sealant/TachoSil (Absorbable Fibrin Sealant Patch) granted on Apr. 2, 20101 to Nycomed Austria GmbH for use as an adjunct to hemostasis in cardiovascular surgery when control of bleeding by standard surgical techniques, such as suture, ligature or cautery, is ineffective or impractical.
110. Immune globulin (SCIG) (Immune Globulin Subcutaneous (Human)—Hizentra; (Vivaglobin is an older, lower-concentration product)) granted on Mar. 4, 2010 to CSL Behring for treatment of primary immunodeficiency.
111. Glucocerebrosidase, rDNA/Shire (velaglucerase alfa; ceramidase, glucosyl-(human HT-1080 cell); human glucosylceramidase (EC 3.2.1.45 or beta-glucocerebrosidase), glycoform alpha; beta-glucocerebrosidase) granted on Feb. 26, 2010 to Shire Pharmaceutical for treatment of Gaucher Disease.
112. Pneumococcal Vaccine (13)-CRM197 (Pneumococcal 13-valent Conjugate Vaccine (Diphtheria CRM197 Protein)—Prevnar 13; Prevenar 13; Streptococcus pnuemoniae capsular antigen-Diphtheria CRM197 protein conjugate vaccine; PCV13) granted on Feb. 24, 2010 to Pfizer (developed by Wyeth) for prevention of Streptococcus pnuemoniae-related disease.
113. Meningococcal Conjugates Vaccine/Novartis (Meningococcal (Groups A, C, Y and W-135) Polysaccharide Diphtheria Toxoid Conjugate Vaccine—Menveo; MenACWY-CRM) granted on Feb. 19, 2010 to Novartis prophylaxis against invasive meningococcal disease.
114. Collagenase (clostridial collagenase for injection—Xiaflex) granted on Feb. 3, 2010 to Auxilium Pharmaceuticals Inc. for the treatment of treatment of Dupuytren's disease.
115. Glucagon-like peptide-1, rDNA (liraglutide—Victoza; Arg34-GLP-1(7-37); GLP-1 (recombinant); NN-2211) granted on Jan. 25, 2010 to Novo Nordisk for the treatment of type 2 diabetes.
116. Interleukin-6 receptor Mab, rDNA (tocilizumab; Actemra; RoActemra; interleukin-6 receptor monoclonal antibody, recombinant; IL-6r Mab) granted on Jan. 8, 2010 to Amgen for the treatment of rheumatoid arthritis (RA).
117. Influenza Vaccine, high dose (Fluzone High-Dose) granted on Dec. 23, 2009 to Sanofi Pasteur for the prevention of influenza in persons 65 years of age of older; full BLA granted; this is a higher-dose (60 Âμg vs. 15 Âμg of each influenza strain HA antigen) formulation of Fluzone, the influenza vaccine most used in the U.S.
118. VWF/Factor VIII Complex (Wilate) granted on Dec. 4, 2009 to Octapharma USA, Inc. for the treatment of von Willebrand disease (VWD).
119. Kallikrein inhibitor, rDNA (ecallantide—Kalbitor; DX-88; kallikrein inhibitor protein, recombinant) granted on Dec. 1, 2009 to Dyax Corp. for the treatment of acute attacks of hereditary angioedema (HAE) in patients 16 years of age and older.
120. Influenza vaccine/Novartis Italy (Agriflu) granted on Nov. 27, 2009 to Novartis for prophylaxis against H1N1 (swine flu) influenza; this (or much the same) conventional inactivated egg-cultured vaccine has long been manufactured at site in Siena, Italy, primarily for European markets.
121. Influenza vaccine, H1N1/GSK Canada granted on Nov. 10, 2009 to ID Biomedical, subsidiary of GlaxoSmithKline (GSK), for prophylaxis against H1N1 (swine flu) influenza; although a new and distinct product, this was approved as a supplemental BLA. (This is an H1N1 analog or biosimilar/biogeneric monovalent version of Influenza vaccine/GSK Canada (FluLaval)).
122. CD20 Mab, human, rDNA (ofatumumab—Arzerra; HuMax-CD20; CD20 monoclonal antibody, human, recombinant) granted on Oct. 26, 2009 to GlaxoSmithKline (and Genmab) for the treatment for chronic lymphocytic leukaemia in patients who have not responded to Campath (alemtuzumab) or fludarabine.
123. Antitrypsin, alpha-1/Talecris (Alpha-1-Proteinase Inhibitor (Human)—Prolastin-C; alpha1-antitrypsin) granted on Oct. 19, 2009 to Talecris Biotherapeutics for the treatment of alpha1-antitrypsin (AAT) deficiency.
124. HPV vaccine, rDNA/GSK (Cervarix MEDI 501; human papilloma virus (HPV) vaccine types 16 and 18 L1 virus-like particles, recombinant) granted on Oct. 16, 2009 to GlaxoSmithKline, Inc. for prophylaxis against cervical cancer in females.
125. C1-esterase inhibitor/CSL (Berinert P; C1INH; C1-INH; complement C1 esterase inhibitor, plasma-derived) granted on Oct. 9, 2009 to CSL Behring LLC for the acute treatment of hereditary angioedema (HAE).
126. IL-12/23 p40 Mab, rDNA (ustekinumab—STELARA; CNTO 1275; interleukin-12 (IL-12) and interleukin-23 (IL-23) p40 subunit monoclonal antibody, human, recombinant) granted on Sep. 25, 2009 to Centocor Ortho Biotech Inc. (Johnson & Johnson) for treatment of moderate to severe plaque psoriasis.
127. Immune Globulin (IGIV)/Bio Products (Immune Globulin Intravenous (Human)—Gammaplex) granted on Sep. 17, 2009 to Bio Products Lab. for the treatment of primary humoral immunodeficiency.
128. Influenza vaccine, H1N1/Novartis granted on Sep. 15, 2009 to Novartis AG for prophylaxis against H1N1 (swine flu) influenza; although a new and distinct product, this was approved as a supplemental BLA.
129. Influenza vaccine, H1N1/Sanofi granted on Sep. 15, 2009 to Sanofi-Pasteur for prophylaxis against H1N1 (swine flu) influenza; although a new and distinct product, this was approved as a supplemental BLA.
130. Influenza vaccine, H1N1/CSL granted on Sep. 15, 2009 to CSL Ltd. for prophylaxis against H1N1 (swine flu) influenza; although a new and distinct product, this was approved as a supplemental BLA.
131. Influenza Vaccine, live rDNA, H1N1 granted on Sep. 15, 2009 to MedImmune (AstraZeneca) for prophylaxis against H1N1 (swine flu) influenza; although a new and distinct product, this was approved as a supplemental BLA.
132. Haemophilus b Vaccine/GSK (Hiberix; Haemophilus influenzae Type b vaccine; Hib vaccine) granted on Aug. 19, 2009 to GlaxoSmithKline (GSK) as Hib vaccine booster dose for children 15 months through 4 years old.
133. Interferon betaser, rDNA/Novartis (Interferon beta-1b—Extavia; 2-166-Interferon beta1 (human fibroblast reduced), 17-L-serine-; interferon betaser, recombinant; NVF233) granted on Aug. 15, 2009 to Novartis Pharmaceuticals for multiple sclerosis indications.
134. Interleukin-1 Mab, rDNA (Canakinumab—Ilaris; interleukin-1 beta monoclonal antibody; ACZ885) granted on Jun. 17, 2009 to Novartis Pharmaceuticals for treatment of Cryopyrin Associated Periodic Syndrome (CAPS).
135. Pancreatic Enzyme/Solvay (pancrelipase—Creon; pancreatic enzymes, porcine-derived) granted on May 1, 2009 to Solvay for treatment of exocrine pancreatic enzyme insufficiency.
136. Botulinum Toxin A/Ipsen (abobotulinumtoxinA—Dysport; Reloxin; Clostridium botulinum toxin type A) BLA granted on Apr. 29, 2009 to Ipsen for treatment of cervical dystonia and a sBLA granted at the same time to Medicis for Reloxin (relabeled Dysport) for treatment of gabellar (frown) lines.
137. TNF Mab, rDNA, human/J&J (Simponi; golimumab; CNTO 148; tumor necrosis factor-alpha human monoclonal antibody, recombinant) granted to Centocor Ortho Biotech Inc./Johnson & Johnson on Apr. 24, 2009 for treatment of three types of immune dysfunction-related arthritis.
138. Japanese Encephalitis Vaccine/Intercell—(Ixiaro; Japanese encephalitis SA14-4-2 virus vaccine; IC51) granted to Intercell Biomedical (with marketing by Novartis) on Mar. 30, 2009 for prevention of Japanese encephalitis disease.
139. Antithrombin III, rDNA (Antithrombin III (Human)—ATryn; rhATIII; AT-III, recombinant transgenic goat) granted to GTC Biotherapeutics, Inc. (Genzyme) on Feb. 6, 2009 for prevention of blood clots in patients with antithrombin deficiency.
140. Fibrinogen/CSL (Fibrinogen Concentrate (Human)—RiaSTAT; Haemocomplettan P; Factor I) granted on Jan. 16, 2009 to CSL Behring for treatment of acute bleeding episodes in patients with congenital fibrinogen deficiency (afibrinogenemia and hypofibrinogenemia).
141. C1-esterase inhibitor/Sanquin (Cinryze; CetorA; C1INH; C1-INH; complement C1 esterase inhibitor, plasma-derived) granted on Oct. 10, 2008 to Lev Pharmaceuticals for routine prophylaxis against angioedema attacks in adolescent and adult patients with hereditary angioedema (HAE; C1 inhibitor deficiency)
142. Insulin aspart, rDNA, 50/50 mix (Novolog Mix 50/50; Biophasic Insulin Aspart 50/50) granted on Aug. 26, 2008 to Novo Nordisk for treatment of diabetes
143. Thrombopoietin peptibody, rDNA (romiplostim—NPLATE; AMG-531; Amgen Megakaryopoiesis Protein 531; thrombopoietin mimetic peptibody, recombinant) granted on Aug. 22, 2008 to Amgen for treatment of adults with chronic immune thrombocytopenic purpura (ITP).
144. DTaP-Hib-Polio Vaccine/Sanofi (Pentacel; ActHIB Reconstituted with Diphtheria and Tetanus Toxoids and Acellular Pertussis Vaccine Adsorbed Combined with Poliovirus Vaccine Inactivated; ActHIB plus Quadracel; Diphtheria & Tetanus Toxoids & Acellular Pertussis Vaccine Adsorbed plus Haemophilus influenzae type b (Hib) vaccine plus Poliovirus Vaccine Inactivated (Human Diploid Cell)), granted on Jun. 20, 2008 to Sanofi Pasteur (As the combination of two previously approved combination vaccines, with the two mixed before administration, BIOPHARMA does not consider this to be a new, distinct/unique product).
145. Interferon alfa-2b, rDNA, PEG-plus Ribavirin (PEGPAK; combination packaging of PEG-Intron and ribavirin) granted Jun. 13, 2008 to Schering-Plough for treatment of chronic hepatitis C.
146. TNF Mab Fab′, rDNA, PEG—(Certolizumab pegol—Cimzia; CDP 870; tumor necrosis factor monoclonal antibody Fab fragment, recombinant—polyethylene glycol (PEG) polymer conjugate) granted on Apr. 23, 2008 to UCB (with U.S. marketing by Bayer Schering) for treatment of refractory Crohn's disease in adults.
147. Rotavirus Vaccine, live/GSK (Rotavirus Vaccine, Live, Oral, Monovalent—Rotarix; RIX-4414) granted on Apr. 4, 2008 to GlaxoSmithKline (GSK) for prevention of rotavirus gastroenteritis in infants.
148. DTaP-IPV/GSK (Diphtheria & Tetanus Toxoids & Acellular Pertussis Vaccine Adsorbed plus Poliovirus Vaccine Inactivated (Human Diploid Cell)—Kinrix) granted on Mar. 24, 2008 to GlaxoSmithKline for pediatric vaccination.
149. Fibrin Sealant/Baxter (Fibrin Sealant, VH S/D 4—Artiss; Fibrin Sealant, Vapor Heated Solvent/Detergent Treated) granted on Mar. 19, 2008 to Baxter Healthcare for use in attaching skin grafts onto burn patients. (This appears to be a next-generation version and replacement for Tisseel Kit VH).
150. Interleukin-1 trap, rDNA (Arcalyst; rilonacept; IL-1 Trap, recombinant) granted on Feb. 27, 2008 to Regeneron Pharmaceuticals Inc. for long term treatment of two Cryopyrin-Associated Periodic Syndromes (CAPS) disorders: Familial Cold Auto-Inflammatory Syndrome (FCAS) and Muckle-Wells Syndrome (MWS).
151. Antihemophilic Factor (Recombinant), Plasma/Albumin Free (Xyntha; recombinant coagulation factor VIII; an updated version of ReFacto, now with no human or animal products used in its manufacture or formulation); full BLA granted to Wyeth on Feb. 21, 2008 for treatment of hemophilia A.
152. Somatropin, rDNA/Cangene (Somatropin (rDNA origin) for Injection—Accretropin)—505(b)(2) follow-on protein NDA granted on Jan. 24, 2008 to Cangene Corp. (marketing by Apotex) for treatment of pediatric growth failure or short stature.
153. Thrombin, rDNA (Recothrom; Thrombin, recombinant; rhThrombin)—granted on Jan. 17, 2008 to ZymoGenetics, Inc. to help halt bleeding from small blood vessels after surgery.
154. EPO, rDNA, PEG—(Continuous Erythropoietin Receptor Activator; Mircera; CERA; epoetin alpha (recombinant), pegylated; methoxy polyethylene glycol-epoetin beta))—granted on Nov. 14, 2007 to Hoffmann-La Roche Inc. for treatment of anemia associated with chronic renal failure in adults.
155. Skin, Cultured/Epicel (Cultured Epidermal Autograft—Epicel; Cultured Autologous Keratinocytes Service; CEA)—HDE granted on Oct. 29, 2007 to Genzyme Corp. for the treatment of life-threatening wounds resulting from severe burns.
156. Influenza Vaccine/CSL (Influenza Virus Vaccine, Trivalent, Types A and B—AFLURIA; Fluvax; Enzira)—granted on Sep. 28, 2007 to CSL Ltd. for prophylaxis against influenza.
157. Smallpox vaccine/Vero—granted on Aug. 31, 2007 to Acambis plc for prophylaxis against smallpox (in the biodefense stockpile).
158. Thrombin/Omrix (Evithrom)—granted on Aug. 27, 2007 to Omrix Biopharmaceuticals for control of bleeding (inaccessible/otherwise untreatable oozing of blood and minor bleeding from capillaries and small venules).
159. Fibrin Sealant/Thermogenesis (CryoSeal Fibrin Sealant System; CryoSeal FS System; cryoprecipitate plus thrombin, autologous); granted on Jul. 26, 2007 to Thermogenesis Corp. for control of bleeding during liver surgery.
160. Immune Globulin (IGIV), liquid/CSL (Immune Globulin Intravenous (Human), 10% Liquid—Privigen); granted on Jul. 26, 2007 to CSL BioPlasma Inc. for treatment of primary immunodeficiency.
161. Influenza Vaccine, H5N1/Sanofi (Influenza Virus Vaccine, H5N1; pandemic influenza vaccine; bird flu vaccine); granted on Apr. 17, 2007 to Sanofi Pasteur Inc. for active immunization of adults at increased risk of exposure to the H5N1 influenza virus (for use in case of a bird flu-related influenza epidemic/pandemic).
162. Somatropin, rDNA/BioPartners (Somatropin (rDNA origin) for Injection—Valtropin; human growth hormone, recombinant); granted on Apr. 19, 2008 to Parexel, a CRO proxy for LG Life Sciences, for treatment of growth deficiencies.
163. Protein C, plasma-derived (Ceprotin); granted on Mar. 27, 2007 to Baxter Healthcare for treatment of severe congenital Protein C deficiency.
164. Complement C5 Mab, rDNA (Eculizumab—Soliris; complement C5 monoclonal antibody, recombinant)—granted on Mar. 16, 2007 to Alexion Pharmaceuticals, Inc. for treatment of paroxysmal nocturnal hemoglobinuria (PNH).
165. Poly-4-hydroxybutyrate, rDNA (TephaFLEX Absorbable Suture; poly-4-hydroxybutyrate; P4HB; poly(4HB); PHA4400)—granted on Feb. 12, 2007 to Tepha, Inc. for use as surgical sutures.
166. Albumin (Human) (one of many Albumin products)—granted on Oct. 17, 2006 to Octapharma Pharmazeutika Produktionsgesm.b.H for the restoration and maintenance of circulating blood volume.
167. Influenza vaccine/ID Biomedical (Influenza Virus Vaccine, Trivalent—FluLaval; Fluviral) granted on Oct. 5, 2006 to GlaxoSmithKline (acquired ID Biomedical) for the active immunization of adults 18 years and older against influenza.
168. EGF receptor Mab, human, rDNA (Panitumumab—Vectibix; ABX-EGF; epidermal growth factor receptor monoclonal antibody, human, recombinant; E7.6.3; rHuMAb-EGFr; transgenic XenoMouse-derived human EGF receptor Mab); granted on Sep. 27, 2006 to Amgen Inc. “for the treatment of patients with epidermal growth factor receptor-(EGFr) expressing metastatic colorectal cancer after disease progression on, or following fluoropyrimidine-, oxaliplatin-, and irinotecan-containing chemotherapy regimens.”
169. Iduronate-2-sulfatase, rDNA (Idursulfase; Elaprase; L-iduronate 2-sulfate sulfatase precursor; recombinant; 12S; chondroitinsulfatase); granted on Jul. 24, 2006 to Shire Pharmaceuticals Group plc (through its acquisition of Transkaryotic Therapies, Inc.) for the treatment of Hunter syndrome (mucopolysaccharidosis II; MPS II).
170. VEGF Mab Fab, rDNA (Lucentis; vascular endothelial growth factor monoclonal antibody fragment, recombinant) granted on Jun. 30, 2006 to Genentech, Inc. for treatment of age-related macular degeneration.
171. HPV vaccine, rDNA/Merck (Quadrivalent Human Papillomavirus (Types 6, 11, 16, 18) Recombinant Vaccine:—Gardasil; human papilloma virus (HPV) types 6, 11, 16 and 18 L1 virus-like proteins (VLPs), recombinant); granted on Jun. 8, 2006 to Merck & Co., Inc. for vaccination in females 9-26 years of age for prevention of diseases caused by human papillomavirus (HPV) types 6, 11, 16, and 18.
172. Somatropin, rDNA/Sandoz (Somatropin (rDNA origin)—Omnitrope; human growth hormone, recombinant) granted on May 30, 2006 to Sandoz, Inc., a subsidiary of Novartis AG, for treatment of growth hormone deficiency.
173. Varicella Virus Vaccine/adult (Zoster vaccine live (Oka/Merck); Zostavax; Varicella virus vaccine for adults); granted on May 25, 2006 to Merck & Co., Inc. for prevention of herpes zoster (shingles) in persons 60 years of age or older.
174. Glucosidase, rDNA (Alglucosidase alfa—Myozyme; Pompase; alpha glucosidase; glucosidase alpha (rhGAA) (recombinant)); granted on Apr. 28, 2006 to Genzyme Corp. for treatment of Pompe disease.
175. Rotavirus Vaccine, rDNA/Merck (Rotavirus Vaccine, Quintavalent—RotaTeq; WC3 pentavalent vaccine); granted on Feb. 3, 2006 to Merck & Co., Inc. for prevention of pediatric rotavirus gastrointestinal disease.
176. Hepatitis B Immune Globulin, i.m./Cangene (Hepatitis B Immune Globulin (Human); HepaGam B); granted on Jan. 27, 2006 to Cangene Corp. for for post-exposure propylaxis following acute exposure to hepatitis B virus.
177. Insulin, rDNA, inhaled/Pfizer (Exubera Insulin, recombinant powder for inhalation); granted on Jan. 27, 2006 to Pfizer, Inc. for the treatment of adults with type 1 and type 2 diabetes.
178. Immune globulin (SCIG) (Vivaglobin); granted on Jan. 9, 2006 to ZLB Behring for treatment of primary immunodeficiency.
179. CTLA4-Ig, rDNA (Orencia; Abatacept; cytotoxic T-lymphocyte-associated antigen 4—Immunoglobulin G1 fragment fusion protein, recombinant; BMS-1886, 1967) granted on Dec. 26, 2005 to Bristol-Myers Squibb Co. for second-line treatment of rheumatoid arthritis in moderate to severe adult patients.
180. Insulin-like Growth Factor-1/IGFBP-3, rDNA (Mecasermin rinfibate—IPLEX; SomatoKine; insulin-like growth factor I—insulin-like growth factor-binding-3 protein complex, recombinant; IGF-1/IGFBP3 complex) granted on Dec. 12, 2005 to Insmed Inc. for the treatment of growth failure in children with severe primary IGF-1 deficiency (Primary IGFD) or with growth hormone (GH) gene deletion who have developed neutralizing antibodies to GH.
181. Hyaluronidase, rDNA (Hylenex; Enhanze SC; Cumulase; Chemophase; rHuPH20; PH-20 hyaluronidase, recombinant human) approval of Hyelex (formerly Enhanze SC) granted on Dec. 5, 2005 to Halozyme Therapeutics Inc. for marketing by Baxter for use as a “spreading agent” to enhance the delivery of local anesthesia, contrast agents, and for subcutaneous fluid replacement (hypodermoclysis).
182. Hyaluronidase, ovine/Primapharm (Hydase) granted on Oct. 25, 2005 to PrimaPharm, Inc. for use as a “spreading agent” to enhance the delivery of local anesthesia, contrast agents, and for subcutaneous fluid replacement (hypodermoclysis).
183. PDGF, rDNA/Bone matrix (Platelet-derived growth factor (PDGF)-BB, recombinant with inorganic bone matrix; rhPDGF-BB; GEM 21S) granted on Oct. 21, 2005 to BioMimetic Therapeutics, Inc. for marketing by Osteohealth Co. (Luitpold Pharmaceuticals, Inc., Sankyo Co., Ltd.) for the treatment of periodontal bone defects and associated gingival recession.
184. Measles Mumps Rubella & Varicella Vaccine (Measles, Mumps, Rubella and Varicella (Oka/Merck) Virus Vaccine Live—ProQuad; M-M-R II plus Varivax vaccine) granted on Sep. 6, 2005 to Merck & Co., Inc. for vaccination against measles, mumps, rubella (German measles) and varicella (chickenpox) in children 12 months to 12 years of age.
185. Influenza Vaccine/GSK Canada (Influenza Virus Vaccine, Trivalent, Types A and B—Fluarix); granted on Aug. 31, 2005 to GlaxoSmithKline Biologicals for prevention of influenza.
186. Influenza vaccine/GSK Germany (Influenza Virus Vaccine, Trivalent—Fluarix; Influsplit SSW; Alpharix); granted on Aug. 31, 2005 to Sachsische Serumwerke AG for marketing by GlaxoSmithKline for prevention of influenza.
187. Insulin-like Growth Factor-1, rDNA/Tercica (Insulin-like Growth Factor-1, recombinant—Increlex; IGF-1) granted on Aug. 31, 2005 to Tercica, Inc. (partnered with Genentech) for the long-term treatment of growth failure in children with severe primary IGF-1 deficiency (Primary IGFD) or with growth hormone (GH) gene deletion who have developed neutralizing antibodies to growth hormone
188. Calcitonin, rDNA (Calcitonin (salmon)—Fortical; calcitonin, recombinant) granted on Aug. 15, 2005 to Unigene, Inc. (marketed by Upsher-Smith Labs.) for the treatment of postmenopausal osteoporosis.
189. Insulin detemir, rDNA—(Insulin detemir, recombinant—Levemir) granted on Jun. 17, 2005 to Novo Nordisk Inc. for the treatment of diabetes mellitus (type 1 and type 2; (a long-acting recombinant insulin analog).
190. dTpa booster/Sanofi (Tetanus Toxoid, Reduced Diphtheria Toxoid and Acellular Pertussis Vaccine, Adsorbed—Adacel; dTpa; Tdap) granted on Jun. 10, 2005 to Aventis Pasteur Ltd. for use as a tetanus, diptheria and pertussis (whooping cough) booster vaccine for those ages 11-64.
191. Arylsulfatase B, rDNA (N-acetylgalactosamine 4-sulfatase—Naglazyme; Aryplase; galsulfase; chondroitinase; rhASB (recombinant))—granted on May 31, 2005 to BioMarin Pharmaceutical Inc. for treatment of mucopolysaccharidosis VI (MPS VI).
192. dTpa booster/GSK (Tetanus Toxoid, Reduced Diphtheria Toxoid and Acellular Pertussis Vaccine, Adsorbed—Boostrix; dTpa; Tdap)—granted on May 3, 2005 to GlaxoSmithKline Biologicals S.A. for use as a tetanus, diptheria and pertussis (whooping cough) booster vaccine for those ages 10-18.
193. Tetanus Toxoid/Chiron (Tetanus Toxoid Concentrate (For Further Manufacturing Use); granted on May 3, 2005 to Chiron Behring GmbH & Co. (Chiron Corp.; merging into Novartis AG) for use as a component of Boostrix combination vaccine (see above entry).
194. Vaccinia Immune Globulin, i.v./Cangene (VIG; VIVIG)—granted on May 3, 2005 to Cangene Corp. for treatment of rare complications of smallpox vaccination (systemic, severe skin or other serious infections due to the live vaccinia virus in current smallpox vaccines).
195. Hyaluronidase, rDNA (Cumulase; Enhanze SC; Chemophase; rHuPH20; PH-20 hyaluronidase, recombinant human)—device approval of Cumulase on Apr. 19, 2005; granted to Halozyme Therapeutics, Inc. for use in in vitro fertilization (IVF) procedures (preparation of oocytes prior to in vitro fertilization).
196. Vaccinia Immune Globulin, i.v./DVC (VIG; VIGIV)—granted on Feb. 18, 2005 to DynPort Vaccine Co. LLC for treatment of rare complications of smallpox vaccination (systemic, severe skin or other serious infections due to the live vaccinia virus in current smallpox vaccines).
197. Thrombin, concentrated (Thrombin (Human) (For Further Manufacturing Use))—granted on Feb. 18, 2005 to Baxter Healthcare Corp. for further manufacture of FloSeal Matrix Hemostatic Sealant, used to control bleeding.
198. Meningococcal Conjugates Vaccine (Menactra; Meningococcal (Groups A, C, Y and W-135) Polysaccharide Diphtheria Toxoid Conjugate Vaccine; MCV-4)—granted on Jan. 14, 2005 to Sanofi Pasteur Inc. for protection against meningococcal disease in adolescents and adults aged 11-55 years.
199. VEGF apatamer, PEG—(Pegaptanib sodium—Macugen; vascular endothelial growth factor/vascular permeability factor (VEGF) aptamer, synthetic oligonucleotide, PEGylated)) granted on Dec. 17, 2004 to Eyetech Pharmaceuticals, Inc. for treatment of neovascular (wet) age-related macular degeneration.
200. Keratinocyte growth factor, rDNA* (Palifermin—Kepivance; des1-23 KGF; 24-163 fibroblast growth factor 7 (human)) granted on Dec. 15, 2004 to Amgen Inc. for treatment of severe oral mucositis (mouth sores) in patients with hematologic cancers undergoing high-dose chemotherapy, followed by a bone marrow transplant.
201. Integrin Mab, rDNA (Tysabri—natalizumab; Antegren; integrin alpha(4) humanized monoclonal antibody) granted on Nov. 24, 2004 to Biogen Idec for treatment of multiple sclerosis (formerly Antegren (trade name), now Tysabri; changed at FDA request).
202. Hyaluronidase, bovine/Amphastar—(Hyaluronidase, bovine—Amphadase) granted on Oct. 24, 2004 to Amphastar Pharmaceuticals, Inc. for use as a “spreading agent,” e.g., as an adjuvant to increase the absorption and dispersion of other injected drugs; for hypodermoclysis; and as an adjunct in subcutaneous urography for improving resorption of radiopaque agents.
203. Enfuvirtide, synthetic (T-20; Fuzeon; pentafuside; DP-178) full approval (upgraded from accelerated approval granted March 2003) granted on Oct. 15, 2004 to Hoffmann-La Roche Inc. for the treatment of HIV-1 infection in in combination with other antiretroviral agents in treatment-experienced patients with evidence of HIV-1 replication despite ongoing antiretroviral therapy (synthetic peptide, not a biopharmaceutical).
204. CD15 Mab-Tc 99m radioconj. (Technetium (99m Tc) fanolesomab; Neutrospec; Leutech; TC99M-labeled CD15 monoclonal antibody)—granted on Jul. 2, 2004 to Palatin Technologies, Inc. for diagnostic imaging of appendicitis (scintigraphic imaging of patients with equivocal signs of appendicitis who are five years of age or older.)
205. Luteinizing hormone, rDNA (Lutropin alfa—Luveris; human luteinizing hormone, recombinant) granted to Scrono, Inc. on May 24, 2004 for infertility treatment (stimulation of follicular development in infertile hypogonadotropic hypogonadal women with profound LH deficiency in combination with FSH (Gonal-f)).
206. Immune Globulin (IGIV)/Octapharma (Octagam; Immune Globulin Intravenous (Human))—granted on May 21, 2004 to Octapharma AG for treatment of primary immune deficiency.
207. Hyaluronidase, ovine (Vitrase; hyaluronate 4-glycanohydrolase) granted on May 4, 2004 to ISTA Pharmaceuticals Inc. for use as a spreading agent to facilitate the dispersion and absorption of drugs, particularly local anesthetics, during ophthalmic surgery; for hypodermoclysis; and as an adjunct in subcutaneous urography for improving resorption of radiopaque agents.
208. Insulin glulisine, rDNA (Apidra; (LysB3, GluB29) insulin; insulin (human), 3B-l-lysine, 29B-l-glutamic acid-, recombinant)—granted on Apr. 16, 2004 to Aventis Pharma for use as a rapid-acting insulin for treatment of diabetes.
209. VEGF Mab, rDNA (Avastin; bevacizumab; vascular endothelial growth factor monoclonal antibody, recombinant)—granted on Feb. 26, 2004 to Genentech, Inc. for use in combination with 5-fluorouracil for treatment of metastatic cancer of the colon or rectum.
210. EGF receptor Mab, rDNA (Cetuximab—Erbitux; IMC-C225; epidermal growth factor receptor monoclonal antibody, recombinant)—granted on Feb. 12, 2004 to ImClone Systems Inc. (for marketing by Bristol-Myers Squibb Co.) for use in combination with irinotecan in the treatment of patients with epidermal growth factor receptor (EGFR)-expressing, metastatic colorectal cancer who are refractory to irinotecan-based chemotherapy, and for monotherapy treatment of patients with EGFR-expressing metastatic colorectal cancer who are intolerant to irinotecan-based chemotherapy.
211. Rho(D) Immune Globulin/ZLB (Rho(D) Immune Globulin Intravenous (Human) Rhophylac)—granted on Feb. 12, 2004 to ZLB Bioplasma AG for antepartum and postpartum prevention of Rho(D) immunization in Rho(D)-negative women.
212. Hyaluronic acid/Anika (ORTHOVISC High Molecular Weight Hyaluronan)—granted on Feb. 5, 2004 to Anika Therapeutics, Inc. for U.S. marketing by Ortho Biotech Products, L.P. (Johnson & Johnson) for the treatment of pain associated with osteoarthritis of the knee.
213. Immune Globulin Intravenous (Human) (Flebogamma)—granted on Dec. 18, 2003 to Instituto Grifols (Probitas Pharma) for treatment of primary immune deficiency.
214. Hyaluronic acid/Medicis (Restylane)—granted on Dec. 12, 2003 to Medicis Pharmaceutical Corp. for correction of moderate to severe facial wrinkles and folds, e.g., nasolabial folds (lines/folds near the nose and mouth).
215. CD11a Mab, rDNA (Efalizumab—Raptiva; CD11a monoclonal antibody, recombinant)—granted on Oct. 27, 2003 to Genentech, Inc. and Xoma Ltd. for the treatment of moderate-to-severe psoriasis in adults who are candidates for systemic or phototherapy.
216. Botulism Immune Globulin Intravenous (Human) (BabyBIG)—granted on Oct. 23, 2003 to the California Department of Health Services for treatment of infant botulism caused by type A or type B Clostridium botulinum.
217. Somatropin, rDNA/Serono (Somatropin (rDNA origin)—Serostim; human growth hormone, recombinant)—full approval granted on Aug. 29, 2003 to Serono Inc. for the treatment of HIV patients with wasting or cachexia.
218. Factor VIII, rDNA, PFM (Antihemophilic Factor (Recombinant), Plasma/Albumin Free Method—Advate; Factor VIII, recombinant; rAHF-PFM)—granted on Jul. 25, 2003 to Baxter Hyland Immuno for treatment of hemophilia A.
219. TNF Receptor-IgG Fc, rDNA (Etancrcept—Enbrel; tumor necrosis factor receptor2-immune globlulin G1 Fc fusion protein, recombinant)—supplemental BLA granted on Jul. 24, 2003 to Amgen Inc. for treatment of active ankylosing spondylitis.
220. Antitrypsin, alpha-1/Aventis (Alpha-1-Proteinase Inhibitor (Human)—Zemaira)—granted on Jul. 8, 2003 to Aventis Behring LLC for chronic augmentation and maintenance therapy in individuals with alpha1-proteinase inhibitor deficiency and evidence of emphysema.
221. CD20 Mab, rDNA—I 131 radioconj. (Iodine I 131 tositumomab—Bexxar; CD20 monoclonal antibody—Iodine I 131 radioimmune conjugate)—granted on Jun. 27, 2003 to Corixa Corp. (formerly Coulter Pharmaceutical) with marketing by GlaxoSmithKline (GSK) for the treatment of patients with CD20 positive, follicular, non-Hodgkin's lymphoma (NHL), with and without transformation, whose disease is refractory to Rituximab and has relapsed following chemotherapy.
222. Immunoglobulin E Mab, rDNA (Omalizumab—Xolair; rhuMab-E25; immunoglobulin E25 monoclonal antibody, recombinant; IgE Mab, rDNA)—granted on Jun. 20, 2003 to Genentech, Inc. (with manufacture by Tanox, Inc. and co-marketing by Novartis Pharmaceutical Corp.) for treatment of moderate-to-severe allergic asthma.
223. Hirudin, desulfato-rDNA/Aventis (Iprivask; Desirudin; Revasc; desulfatohirudin; hirudin, desulfato-recombinant)—granted on Apr. 3, 2003 to Aventis Pharma for approved for the prophylaxis of deep vein thrombosis, which may lead to pulmonary embolism, in patients undergoing elective hip replacement surgery.
224. Influenza Vaccine, live rDNA, frozen (FluMist)—granted on Jun. 17, 2003 to Medimmune Vaccines, Inc. (subsidiary of MedImmune, Inc.) for influenza prophylaxis in healthy persons from 5-50 year of age.
225. Iduronidase, rDNA (laronidase; Aldurazyme; alpha-L-iduronidase)—granted on Apr. 30, 2003 to Biomarin Pharmaceutical Inc. (and Genyzme Corp.) for treatment of Mucopolysaccharidosis I (MPS I).
226. Galactosidase, beta rDNA (Agalsidase beta—Fabrazyme; alpha-galactosidase A) granted on Apr. 24, 2003 to Genzyme Corp. for treatment of Fabry disease.
227. Somatropin antagonist, PEG−, rDNA (Pegvisomant—Somavert; somatropin antagonist, pegylated, recombinant)—granted on Mar. 25, 2003 to Pharmacia Corp. for treatment of acromegaly.
228. Enfuvirtide, synthetic (T-20; Fuzeon; pentafuside; DP-178)—granted on Mar. 15, 2003 to Hoffmann-La Roche Inc. for treatment of HIV-infection.
229. LFA-3/IgG1, rDNA (Alefacept; Amevive; leukocyte function-associated antigen-3/immune globulin G (IgG) fusion protein, recombinant)—granted on Jan. 30, 2003 to Biogen Corp. for treatment of moderate-to-severe chronic plaque psoriasis.
230. Antitrypsin, alpha-1/Baxter (alpha-1 Proteinase Inhibitor (Human); Aralast; alpha-1 antitrypsin; AAT; A1P1)—granted on Jan. 9, 2003 to Alpha Therapeutic Corp. (for marketing by Baxter) for enzyme replacement therapy in patients with heredity emphysema (AAT deficiency).
231. TNF Mab, rDNA, human (Adalimumab; Humira; D2E7; tumor necrosis factor-alpha human monoclonal antibody)—granted on Dec. 30, 2002 to Abbott Laboratories for treatment of rheumatoid arthritis.
232. DTaP & Hepatitis B & Polio Vaccine (Diphtheria and Tetanus Toxoids and Acellular Pertussis Adsorbed, Hepatitis B (Recombinant) and Inactivated Poliovirus Vaccine Combined; Pediarix; Infanrix+Engerix-B+IPOL)—a combination vaccine; approval granted on Dec. 13, 2002 to GlaxoSmithKline Inc. for prevention of diphtheria, tetanus, pertussis (whooping cough), hepatitis B, and polio; Poliovirus vaccine (mixture of three inactivated strains) is the only component not previously approved.
233. Parathyroid hormone (1-34), rDNA (teriparatide (rDNA origin); Forteo; LY333334; parathyroid hormone (1-34), recombinant)—granted on Nov. 26, 2002 to Eli Lilly & Co. for treatment of osteoporosis.
234. Interferon alfa-2a, rDNA, PEG—(Peginterferon alfa-2a; Pegasys; interferon alpha-2a, recombinant, pegylated)—granted on Oct. 16, 2002 to Hoffmann-La Roche Inc. for first-line treatment of chronic hepatitis C.
235. Urate oxidase, rDNA (uric acid oxidase, recombinant; rasburicase; re-Uox; Elitek; Fasturtec)—granted on Jul. 16, 2002 to Sanofi-Synthelabo for control of plasma uric acid levels (hyperuricaemia) in pediatric patients receiving cancer chemotherapy resulting in tumor lysis and elevation of uric acid.
236. Bone morphogenic protein-2, rDNA (bone morphogenetic protein-2, recombinant; BMP-2; INFUSE Bone Graft)—PMA granted on Jul. 2, 2002 to Medtronic Sofamor Danek using recombinant bmp-2 (from Genetics Institue/Wyeth) as part of the INFUSE Bone Graft/LT-CAGE Lumbar Tapered Fusion Device for treatment of certain types of spinal degenerative disc disease (lumbar spinal fusion).
237. DTaP Vaccine/Aventis Canada Diphtheria and Tetanus Toxoids and Acellular Pertussis Vaccine Adsorbed (DTaP) (DAPTACEL)—BLA granted on May 14, 2002 to Aventis Pasteur, Ltd. for the first 4 doses of the diphtheria and tetanus toxoids and pertussis vaccination series administered to infants and children aged 6 weeks up to 7 years.
238. Botulinum Toxin Type A Purified Neurotoxin Complex (BOTOX COSMETIC)—supplemental BLA granted on Apr. 12, 2002 to Allergan, Inc. for temporary improvement in the appearance of moderate to severe glabellar lines (“frown lines”) associated with corrugator and/or procerus muscle activity in adult patients<65 years of age.
239. Secretin, synthetic (SecreFlo; porcine secretin) granted on Apr. 5, 2002 to Repligen Corp. for diagnosis of gastrinoma (tumors that secrete gastrin) and pancreatic disorders.
240. Interferon beta-1a, rDNA/Serono (Rebif)—granted on Mar. 7, 2002 to Serono, Inc. for treatment of relapsing forms of multiple sclerosis.
241. CD20 Mab/Y-90 radioconj. (Ibritumomab Tiuxetan; Zevalin; a CD20 monoclonal antibody-chelating group conjugate)—granted on Feb. 29, 2002 to IDEC Pharmaceuticals Corp. for treatment of B-cell non-Hodgkin's lymphoma; regimen includes Rituximab, Indium-111 Ibritumomab Tiuxetan, and Yttrium-90 Ibritumomab Tiuxetan.
242. G-CSF, rDNA, PEG—(Pegfilgrastim; Neulasta; pegylated granulocyte-colony stimulating factor)—granted on Jan. 31, 2002 to Amgen, Inc. for treatment of febrile neutropenia in patients receiving chemotherapy for non-myeloid malignancies.

### I-B Container

Optionally in any embodiment, the primary drug container can be a syringe which has Plunger Breakloose Force represented by Fᵢ of less than 15N and Plunger Glide Force represented by Fₘ less than 5N while the number of particles greater than 2 micron is less than 2000 during the two year shelf life; Optionally, the syringe containing a monoclonal antibody is stored at a temperature ranging from 4° C. to 25° C.

Optionally in any embodiment, the primary drug container can be a syringe, cartridge, or vial, optionally a delivery device, optionally a prefilled syringe or prefilled cartridge.

Optionally in any embodiment, the primary drug container can be made of glass or thermoplastic, preferably injection-moldable thermoplastic, optionally selected from COC (cyclic olefin copolymer), COP (cyclic olefin polymer), polypropylene, PET (polyethylene terephthalate), polycarbonate, polystyrene, or combinations of any two or more of these. COP containers are particularly contemplated.

The container should be manufactured in such a way that it has a low intrinsic particle count. For example, the following expedients may be useful:

ISO Class 7 Manufacturing Rooms, monitored and controlled

To ensure low particle load and avoid bioburden contamination, the open product is processed under additional HEPA-air flow for part handling to achieve ISO Class 5 for particles

Minimize manual part handling using automated molding and coating cells.

In process controls at molding and coating to mitigate particle generation and cosmetic defects

Empty container inspection—Automated, on line particle inspection for empty containers. Detect 50 μm particles with false positives <5%.

Optionally in any embodiment, the secondary packaging for the container that is Tyvek-free

### I-C Drug-Contact Coating

Optionally in any embodiment, the drug-contact coating consists essentially of SiOₓC_{y}H_{z}, in which
x is between 0.5 and 2.4, optionally between 1.3 and 1.9, as measured by x-ray photoelectron spectroscopy (XPS),
y is between 0.6 and 3, optionally between 0.8 and 1.4, as measured by XPS; and
z is between 2 and 9, optionally between 2 and 6, as measured by Rutherford backscattering.

Optionally in any embodiment, the drug-contact coating thickness is between 5 nm and 1000 nm, optionally between 10 nm and 500 nm, optionally between 10 nm and 300 nm.

Optionally in any embodiment, the drug contact coating is lubricious.

Optionally in any embodiment, the drug contact coating is a solid lubricious coating.

Optionally in any embodiment, the drug contact coating is a pH protective coating.

Optionally in any embodiment, the drug contact coating is a lubricity coating of SiOₓC_{y}H_{z}, in which x is 0.5-2.4, y is 0.6-3, x and y being measured by x-ray photoelectron spectroscopy (XPS), and z is 2-9, z being measured by Rutherford backscattering analysis, applied by plasma enhanced chemical vapor deposition (PECVD). A “lubricity coating” is defined as a coating that reduces the breakloose force or maintenance force necessary to advance the plunger in the barrel of a syringe, compared to the breakloose force or maintenance force necessary in a syringe made under the same conditions but lacking the lubricity coating. This is the fourth coating of the quadlayer coating described in this specification. The nature and application of lubricity coatings is described in WO2013/071138, which is incorporated here by reference. One contemplated lubricity coating, sometimes referred to as l-OMCTS, is a PECVD coating having the molecular formula SiOₓC_{y}H_{z}, in which x is 0.5-2.4, y is 0.6-3, x and y being measured by x-ray photoelectron spectroscopy (XPS), and z is 2-9, z being measured by Rutherford backscattering analysis, made using octamethylcyclotetrasiloxane (OMCTS) as the organosilicon precursor.

Optionally in any embodiment, the drug contact coating is a gas barrier coating, an extractable barrier coating, or both.

Optionally in any embodiment, the drug contact coating is plasma-treated to provide reduced protein adhesion.

Optionally in any embodiment, the drug contact coating or treatment increases protein adhesion without releasing these adhered proteins back to the solution and thus does not increase the number of particles in the container or even reduces the number of particles in the container during prolonged shelf life time.

Optionally in any embodiment, the drug container is provided with a multilayer PECVD coating, of which the final coating is the drug-contact coating. Optionally in any embodiment, the multilayer coating contemplated here can be a trilayer coating including an adhesion or tie coating or layer of SiOₓC_{y}H_{z} as described in this specification, a barrier coating or layer of SiOₓ as described in this specification, and a pH protective coating or layer, in this case the drug contact layer, of SiOₓC_{y}H_{z} as described in this specification, each applied by plasma enhanced chemical vapor deposition (PECVD). Optionally in any embodiment, the multilayer coating contemplated here can be a quadlayer coating including an adhesion or tie coating or layer of SiOₓC_{y}H_{z}, a barrier coating or layer of SiOₓ, a pH protective coating or layer of SiOₓC_{y}H_{z}, and a lubricity coating or layer of l-OMCTS, in this case the drug contact layer, each applied by plasma enhanced chemical vapor deposition (PECVD), optionally in the manner described elsewhere in this specification.

Optionally in any embodiment, the drug contact coating is chemically homogeneous. “Homogeneous” is defined for a PECVD drug contact coating as having an atomic % standard deviation in each element (Si, C and O) of SiOₓC_{y}H_{z} in different locations of a given container of less than 5%, alternatively less than 4%, alternatively less than 3%, alternatively less than 2%, alternatively less than 1%, determined x-ray photoelectron spectroscopy (XPS) analysis.

Optionally in any embodiment, the drug contact coating is free of fluid lubricant.

Optionally in any embodiment, the drug contact coating is free of silicone oil.

### I-D Additional PECVD Coatings

Optionally in any embodiment, the primary drug container also has a barrier coating or layer providing a barrier improvement factor of at least 3, optionally at least 5, optionally at least 10, optionally at least 20, optionally at least 50.

Optionally in any embodiment, the primary drug container also has an adhesion coating or layer disposed between the internal surface and the PECVD drug-contact coating.

Optionally in any embodiment, the primary drug container also has a pH protective coating or layer for pH 5-9. Optionally in any embodiment, the pH protective coating has a silicon dissolution rate of less than 1 μg/day (microgram per day), alternatively less than 0.5 μg/day, alternatively less than 0.4 μg/day, alternatively less than 0.3 μg/day, alternatively less than 0.2 μg/day, when the lumen contains water for injection, alternatively a drug, alternatively a pH 5-8 aqueous, phosphate buffered test solution.

Optionally in any embodiment, the drug contact coating consists essentially of a PECVD SiOₓC_{y}H_{z} coating or layer, in which
x is between 0.5 and 2.4, optionally between 1.3 and 1.9, as measured by x-ray photoelectron spectroscopy (XPS),
y is between 0.6 and 3, optionally between 0.8 and 1.4, as measured by XPS; and
z is between 2 and 9, optionally between 2 and 6, as measured by Rutherford backscattering.

Optionally in any embodiment, the primary drug container further comprises a PECVD SiOₓ barrier coating or layer between the drug contact coating and the internal surface and a PECVD SiOₓC_{y}H_{z} adhesive coating or layer between the barrier coating or layer and the internal surface.

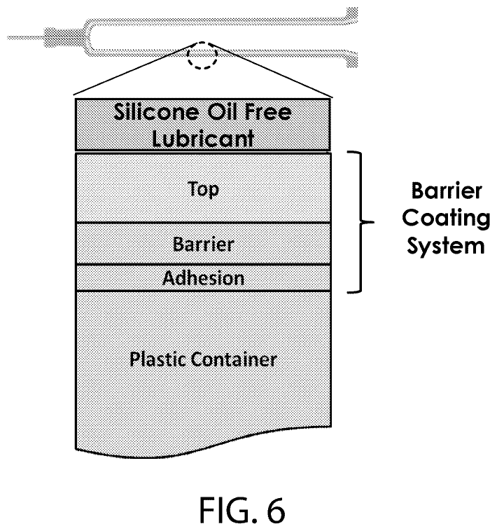

Suitable coatings, coating sets, and surface treatments are illustrated in FIG. 6 and further discussed here.

### Tie Coating or Layer

Referring to FIG. 6, the tie coating or layer is provided, sometimes referred to as an adhesion coating or layer. The tie coating or layer optionally functions to improve adhesion of a barrier coating or layer to a substrate, in particular a thermoplastic substrate, although a tie layer can be used to improve adhesion to a glass substrate or to another coating or layer.

Optionally, the tie coating or layer improves adhesion of the barrier coating or layer to the substrate or wall. For example, the tie coating or layer, also referred to as an adhesion layer or coating, can be applied to the substrate and the barrier layer can be applied to the adhesion layer to improve adhesion of the barrier layer or coating to the substrate. Optionally, the adhesion or tie coating or layer is also believed to relieve stress on the barrier coating or layer, making the barrier layer less subject to damage from thermal expansion or contraction or mechanical shock.

Optionally, the tie coating or layer applied under a barrier coating or layer can improve the function of a pH protective coating or layer applied over the barrier coating or layer.

Optionally, the adhesion or tie coating or layer is also believed to decouple defects between the barrier coating or layer and the COP substrate. This is believed to occur because any pinholes or other defects that may be formed when the adhesion or tie coating or layer is applied tend not to be continued when the barrier coating or layer is applied, so the pinholes or other defects in one coating do not line up with defects in the other. Optionally, the adhesion or tie coating or layer has some efficacy as a barrier layer, so even a defect providing a leakage path extending through the barrier coating or layer is blocked by the adhesion or tie coating or layer.

Optionally, the tie coating or layer comprises SiOₓC_{y}H_{z} or SiNₓC_{y}H_{z}, preferably can be composed of, comprise, or consist essentially of SiOₓC_{y}H_{z}, wherein x is from about 0.5 to about 2.4, y is from about 0.6 to about 3, and; and z is between 2 and 9, optionally between 2 and 6, as measured by Rutherford backscattering. The atomic ratios of Si, O, and C in the tie coating or layer 289 optionally can be:
Si 100:O 50-150:C 90-200 (i.e. x=0.5 to 1.5, y=0.9 to 2);
Si 100:O 70-130:C 90-200 (i.e. x=0.7 to 1.3, y=0.9 to 2)
Si 100:O 80-120:C 90-150 (i.e. x=0.8 to 1.2, y=0.9 to 1.5)
Si 100:O 90-120:C 90-140 (i.e. x=0.9 to 1.2, y=0.9 to 1.4), or
Si 100:O 92-107:C 116-133 (i.e. x=0.92 to 1.07, y=1.16 to 1.33).

The atomic ratio can be determined by XPS. Taking into account the H atoms, which are not measured by XPS, the tie coating or layer 289 may thus in one aspect have the formula Si_{w}OₓC_{y}H_{z} (or its equivalent SiOₓC_{y}), for example where w is 1, x is from about 0.5 to about 2.4, y is from about 0.6 to about 3, and z is from about 2 to about 9. Typically, tie coating or layer 289 would hence contain 36% to 41% carbon normalized to 100% carbon plus oxygen plus silicon.

Optionally, the tie coating or layer can be similar or identical in composition with the pH protective coating or layer 286 described elsewhere in this specification, although this is not a requirement.

Optionally, the tie coating or layer 289 is on average between 5 and 200 nm (nanometers), optionally between 5 and 100 nm, optionally between 5 and 20 nm thick. These thicknesses are not critical. Commonly but not necessarily, the tie coating or layer 289 will be relatively thin, since its function is to change the surface properties of the substrate.

The tie coating or layer 289 has an interior surface facing the lumen 212 and an outer surface facing the wall 214 interior surface. Optionally, the tie coating or layer 286 is at least coextensive with the barrier coating or layer. Optionally, the tie coating or layer is applied by PECVD, for example of a precursor feed comprising octamethylcyclotetrasiloxane (OMCTS), tetramethyldisiloxane (TMDSO), or hexamethyldisiloxane (HMDSO).

### Barrier Coating or Layer

Referring to FIG. 6, a barrier coating or layer optionally can be deposited by plasma enhanced chemical vapor deposition (PECVD) or other chemical vapor deposition processes on the vessel of a pharmaceutical package, for example a thermoplastic package, to prevent oxygen, carbon dioxide, or other gases from entering the vessel, the barrier coating optionally being effective to reduce the ingress of atmospheric gas into the lumen compared to an uncoated vessel, and/or to prevent leaching of the pharmaceutical material into or through the package wall.

The barrier coating or layer optionally can be applied directly or indirectly to the thermoplastic wall to lower the oxygen transmission rate and/or moisture transmission rate.

The barrier coating or layer optionally can be silicon oxide, titanium oxide or zinc oxide, applied directly or indirectly to the thermoplastic wall made of COP to lower the oxygen transmission rate and/or moisture transmission rate.

The barrier coating or layer optionally can be applied directly or indirectly to the thermoplastic wall of a plastic container (for example an adhesion or tie coating or layer can be interposed between them) so that in the filled pharmaceutical package or other vessel the barrier coating or layer is located between the inner or interior surface of the wall and the lumen that is adapted to contain a fluid to be stored. The barrier coating or layer of SiOₓ is supported by the thermoplastic wall of the plastic container. The barrier coating or layer as described elsewhere in this specification, or in U.S. Pat. No. 7,985,188, can be used in any embodiment.

The barrier layer optionally is characterized as an “SiOₓ” coating, and contains silicon, oxygen, and optionally other elements, in which x, the ratio of oxygen to silicon atoms, is from about 1.5 to about 2.9, or 1.5 to about 2.6, or about 2. One suitable barrier composition is one where x is 2.3, for example.

Optionally, the barrier coating or layer 288 is from 2 to 1000 nm thick, optionally from 4 nm to 500 nm thick, optionally between 10 and 200 nm thick, optionally from 20 to 200 nm thick, optionally from 20 to 30 nm thick, and comprises SiOₓ, wherein x is from 1.5 to 2.9. The barrier coating or layer 288 of SiOₓ has an interior surface 220 facing the lumen 212 and an outer surface 222 facing the interior surface of the tie coating or layer 289. For example, the barrier coating or layer such as 288 of any embodiment can be applied at a thickness of at least 2 nm, or at least 4 nm, or at least 7 nm, or at least 10 nm, or at least 20 nm, or at least 30 nm, or at least 40 nm, or at least 50 nm, or at least 100 nm, or at least 150 nm, or at least 200 nm, or at least 300 nm, or at least 400 nm, or at least 500 nm, or at least 600 nm, or at least 700 nm, or at least 800 nm, or at least 900 nm. The barrier coating or layer can be up to 1000 nm, or at most 900 nm, or at most 800 nm, or at most 700 nm, or at most 600 nm, or at most 500 nm, or at most 400 nm, or at most 300 nm, or at most 200 nm, or at most 100 nm, or at most 90 nm, or at most 80 nm, or at most 70 nm, or at most 60 nm, or at most 50 nm, or at most 40 nm, or at most 30 nm, or at most 20 nm, or at most 10 nm, or at most 5 nm thick.

Ranges of from 4 nm to 500 nm thick, optionally from 7 nm to 400 nm thick, optionally from 10 nm to 300 nm thick, optionally from 20 nm to 200 nm thick, optionally from 20 to 30 nm thick, optionally from 30 nm to 100 nm thick are contemplated. Specific thickness ranges composed of any one of the minimum thicknesses expressed above, plus any equal or greater one of the maximum thicknesses expressed above, are expressly contemplated.

The thickness of the SiOₓ or other barrier coating or layer can be measured, for example, by transmission electron microscopy (TEM), and its composition can be measured by X-ray photoelectron spectroscopy (XPS).

Optionally, the barrier coating or layer is effective to reduce the ingress of atmospheric gas into the lumen compared to a vessel without a barrier coating or layer. Optionally, the barrier coating or layer provides a barrier to oxygen that has permeated the wall. Optionally, the barrier coating or layer is a barrier to extraction of the composition of the wall by the contents of the lumen.

### pH Protective Coating or Layer

Certain barrier coatings or layers such as SiOₓ as defined here have been found to have the characteristic of being subject to being measurably diminished in barrier improvement factor in less than six months as a result of attack by certain relatively high pH contents of the coated vessel as described elsewhere in this specification, particularly where the barrier coating or layer directly contacts the contents. The inventors have found that barrier layers or coatings of SiOₓ are eroded or dissolved by some fluids, for example aqueous compositions having a pH above about 5. Since coatings applied by chemical vapor deposition can be very thin—tens to hundreds of nanometers thick—even a relatively slow rate of erosion can remove or reduce the effectiveness of the barrier layer in less time than the desired shelf life of a product package. This is particularly a problem for aqueous fluid pharmaceutical compositions, since many of them have a pH of roughly 7, or more broadly in the range of 4 to 8, alternatively from 5 to 9, similar to the pH of blood and other human or animal fluids. The higher the pH of the pharmaceutical preparation, the more quickly it erodes or dissolves the SiOₓ coating. Optionally, this problem can be addressed by protecting the barrier coating or layer 288, or other pH sensitive material, with a pH protective coating or layer 286.

The pH protective coating or layer optionally provides protection of the underlying barrier coating or layer against contents of the vessel having a pH from 4 to 8, including where a surfactant is present. For a prefilled pharmaceutical package that is in contact with the contents of the lumen from the time it is manufactured to the time it is used, the pH protective coating or layer optionally prevents or inhibits attack of the barrier coating or layer sufficiently to maintain an effective oxygen barrier over the intended shelf life of the prefilled syringe. The rate of erosion, dissolution, or leaching (different names for related concepts) of the pH protective coating or layer, if directly contacted by a fluid, is less than the rate of erosion of the barrier coating or layer, if directly contacted by the fluid having a pH of from 5 to 9. The pH protective coating or layer is effective to isolate a fluid having a pH between 5 and 9 from the barrier coating or layer, at least for sufficient time to allow the barrier coating to act as a barrier during the shelf life of the pharmaceutical package or other vessel.

The inventors have further found that certain pH protective coatings or layers of SiOₓC_{y}H_{z} or SiNₓC_{y}H_{z} formed from polysiloxane precursors as the top layer shown in FIG. 6, which pH protective coatings or layers have a substantial organic component, do not erode quickly when exposed to fluids, and in fact erode or dissolve more slowly than Type 1 borosilicate glass when the fluids have pHs within the range of 4 to 8 or 5 to 9. For example, at pH 8, the dissolution rate of a pH protective coating or layer made from the precursor octamethylcyclotetrasiloxane, or OMCTS, is quite slow. These pH protective coatings or layers of SiOₓC_{y}H_{z} or SiNₓC_{y}H_{z} can therefore be used to cover a barrier layer of SiOₓ, retaining the benefits of the barrier layer by protecting it from the fluid in the pharmaceutical package. The protective layer is applied over at least a portion of the SiOₓ layer to protect the SiOₓ layer from contents stored in a vessel, where the contents otherwise would be in contact with the SiOₓ layer.

Although the present invention does not depend upon the accuracy of the following theory, it is further believed that effective pH protective coatings or layers for avoiding erosion can be made from siloxanes and silazanes as described in this disclosure. SiOₓC_{y}H_{z} or SiNₓC_{y}H_{z} coatings deposited from cyclic siloxane or linear silazane precursors, for example octamethylcyclotetrasiloxane (OMCTS), are believed to include intact cyclic siloxane rings and longer series of repeating units of the precursor structure. These coatings are believed to be nanoporous but structured and hydrophobic, and these properties are believed to contribute to their success as pH protective coatings or layers, and also protective coatings or layers. This is shown, for example, in U.S. Pat. No. 7,901,783. SiOₓC_{y}H_{z} or SiNₓC_{y}H_{z} coatings also can be deposited from linear siloxane or linear silazane precursors, for example hexamethyldisiloxane (HMDSO) or tetramethyldisiloxane (TMDSO).

The inventors offer the following theory of operation of the pH protective coating or layer described here. The invention is not limited by the accuracy of this theory or to the embodiments predictable by use of this theory.

The dissolution rate of the SiOₓ barrier layer is believed to be dependent on SiO bonding within the layer. Oxygen bonding sites (silanols) are believed to increase the dissolution rate.

It is believed that the OMCTS-based pH protective coating or layer bonds with the silanol sites on the SiOₓ barrier layer to “heal” or passivate the SiOₓ surface and thus dramatically reduces the dissolution rate. In this hypothesis, the thickness of the OMCTS layer is not the primary means of protection—the primary means is passivation of the SiOₓ surface. It is contemplated that a pH protective coating or layer as described in this specification can be improved by increasing the crosslink density of the pH protective coating or layer.

The pH protective coating or layer optionally is effective to keep the barrier coating or layer at least substantially undissolved as a result of attack by the fluid 218 for a period of at least six months.

The pH protective coating or layer optionally can prevent or reduce the precipitation of a compound or component of a composition in contact with the pH protective coating or layer, in particular can prevent or reduce insulin precipitation or blood clotting, in comparison to the uncoated surface and/or to a barrier coated surface using HMDSO as precursor.

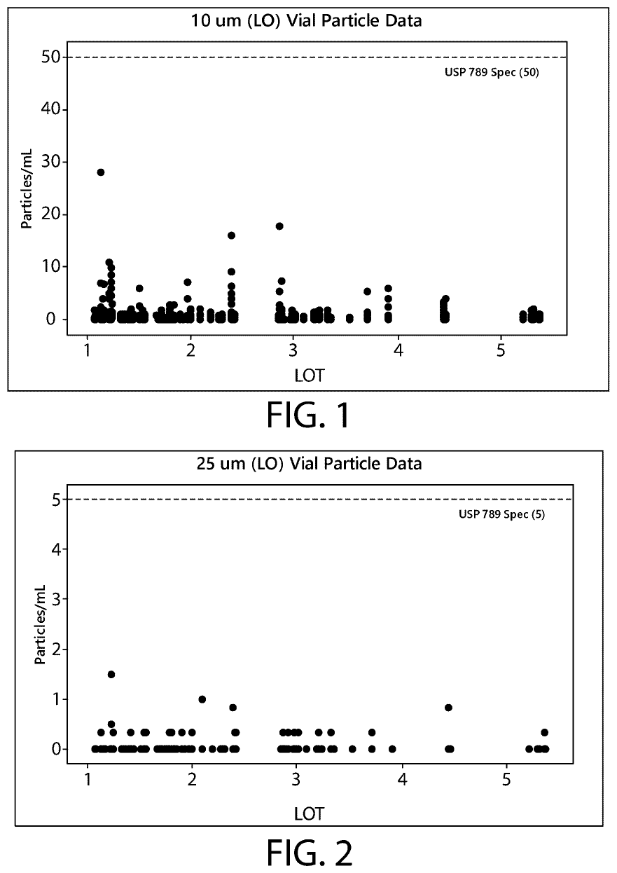

Referring to FIGS. 1 and 2, the pH protective coating or layer 286 can be composed of, comprise, or consist essentially of Si_{w}OₓC_{y}H_{z} (or its equivalent SiOₓC_{y}) or Si_{w}NₓC_{y}H_{z} or its equivalent SiNₓC_{y}), each as defined previously, preferably SiOₓC_{y}H_{z}, wherein x is from about 0.5 to about 2.4 and y is from about 0.6 to about 3; and z is between 2 and 9, optionally between 2 and 6, as measured by Rutherford backscattering. The atomic ratios of Si, O, and C in the pH protective coating or layer 286 optionally can be:
Si 100:O 50-150:C 90-200 (i.e. x=0.5 to 1.5, y=0.9 to 2);
Si 100:O 70-130:C 90-200 (i.e. x=0.7 to 1.3, y=0.9 to 2)
Si 100:O 80-120:C 90-150 (i.e. x=0.8 to 1.2, y=0.9 to 1.5)
Si 100:O 90-120:C 90-140 (i.e. x=0.9 to 1.2, y=0.9 to 1.4), or
Si 100:O 92-107:C 116-133 (i.e. x=0.92 to 1.07, y=1.16 to 1.33) or
Si 100:O 80-130:C 90-150.

Alternatively, the pH protective coating or layer can have atomic concentrations normalized to 100% carbon, oxygen, and silicon, as determined by X-ray photoelectron spectroscopy (XPS) of less than 50% carbon and more than 25% silicon. Alternatively, the atomic concentrations are from 25 to 45% carbon, 25 to 65% silicon, and 10 to 35% oxygen. Alternatively, the atomic concentrations are from 30 to 40% carbon, 32 to 52% silicon, and 20 to 27% oxygen. Alternatively, the atomic concentrations are from 33 to 37% carbon, 37 to 47% silicon, and 22 to 26% oxygen.

Optionally, the atomic concentration of carbon in the pH protective coating or layer, normalized to 100% of carbon, oxygen, and silicon, as determined by X-ray photoelectron spectroscopy (XPS), can be greater than the atomic concentration of carbon in the atomic formula for the organosilicon precursor. For example, embodiments are contemplated in which the atomic concentration of carbon increases by from 1 to 80 atomic percent, alternatively from 10 to 70 atomic percent, alternatively from 20 to 60 atomic percent, alternatively from 30 to 50 atomic percent, alternatively from 35 to 45 atomic percent, alternatively from 37 to 41 atomic percent.

Optionally, the atomic ratio of carbon to oxygen in the pH protective coating or layer can be increased in comparison to the organosilicon precursor, and/or the atomic ratio of oxygen to silicon can be decreased in comparison to the organosilicon precursor.

Optionally, the pH protective coating or layer can have an atomic concentration of silicon, normalized to 100% of carbon, oxygen, and silicon, as determined by X-ray photoelectron spectroscopy (XPS), less than the atomic concentration of silicon in the atomic formula for the feed gas. For example, embodiments are contemplated in which the atomic concentration of silicon decreases by from 1 to 80 atomic percent, alternatively by from 10 to 70 atomic percent, alternatively by from 20 to 60 atomic percent, alternatively by from 30 to 55 atomic percent, alternatively by from 40 to 50 atomic percent, alternatively by from 42 to 46 atomic percent.

As another option, a pH protective coating or layer is contemplated in any embodiment that can be characterized by a sum formula wherein the atomic ratio C:O can be increased and/or the atomic ratio Si:O can be decreased in comparison to the sum formula of the organosilicon precursor.

The atomic ratio of Si:O:C or Si:N:C can be determined by XPS (X-ray photoelectron spectroscopy). Taking into account the H atoms, the pH protective coating or layer may thus in one aspect have the formula Si_{w}OₓC_{y}H_{z}, or its equivalent SiOₓC_{y}, for example where w is 1, x is from about 0.5 to about 2.4, y is from about 0.6 to about 3, and z is from about 2 to about 9, optionally from about 2 to about 6.

The thickness of the pH protective coating or layer as applied optionally is between 10 and 1000 nm; alternatively from 10 nm to 900 nm; alternatively from 10 nm to 800 nm; alternatively from 10 nm to 700 nm; alternatively from 10 nm to 600 nm; alternatively from 10 nm to 500 nm; alternatively from 10 nm to 400 nm; alternatively from 10 nm to 300 nm; alternatively from 10 nm to 200 nm; alternatively from 10 nm to 100 nm; alternatively from 10 nm to 50 nm; alternatively from 20 nm to 1000 nm; alternatively from 50 nm to 1000 nm; alternatively from 50 nm to 800 nm; optionally from 50 to 500 nm; optionally from 100 to 200 nm; alternatively from 100 nm to 700 nm; alternatively from 100 nm to 200 nm; alternatively from 300 to 600 nm. The thickness does not need to be uniform throughout the vessel, and will typically vary from the preferred values in portions of a vessel.

The pH protective coating or layer can have a density between 1.25 and 1.65 g/cm³, alternatively between 1.35 and 1.55 g/cm³, alternatively between 1.4 and 1.5 g/cm³, alternatively between 1.4 and 1.5 g/cm³, alternatively between 1.44 and 1.48 g/cm³, as determined by X-ray reflectivity (XRR). Optionally, the organosilicon compound can be octamethylcyclotetrasiloxane and the pH protective coating or layer can have a density which can be higher than the density of a pH protective coating or layer made from HMDSO as the organosilicon compound under the same PECVD reaction conditions.

The pH protective coating or layer optionally can have an RMS surface roughness value (measured by AFM) of from about 5 to about 9, optionally from about 6 to about 8, optionally from about 6.4 to about 7.8. The Rₐ surface roughness value of the pH protective coating or layer, measured by AFM, can be from about 4 to about 6, optionally from about 4.6 to about 5.8. The Rₘₐₓ surface roughness value of the pH protective coating or layer, measured by AFM, can be from about 70 to about 160, optionally from about 84 to about 142, optionally from about 90 to about 130.

The interior surface of the pH protective optionally can have a contact angle (with distilled water) of from 900 to 110°, optionally from 80° to 120°, optionally from 700 to 130°, as measured by Goniometer Angle measurement of a water droplet on the pH protective surface, per ASTM D7334—08 “Standard Practice for Surface Wettability of Coatings, Substrates and Pigments by Advancing Contact Angle Measurement.”

Optionally an FTIR absorbance spectrum of the pH protective coating or layer 286 of any embodiment has a ratio greater than 0.75 between the maximum amplitude of the Si—O—Si symmetrical stretch peak normally located between about 1000 and 1040 cm-1, and the maximum amplitude of the Si—O—Si assymetric stretch peak normally located between about 1060 and about 1100 cm-1. Alternatively in any embodiment, this ratio can be at least 0.8, or at least 0.9, or at least 1.0, or at least 1.1, or at least 1.2. Alternatively in any embodiment, this ratio can be at most 1.7, or at most 1.6, or at most 1.5, or at most 1.4, or at most 1.3. Any minimum ratio stated here can be combined with any maximum ratio stated here, as an alternative embodiment of the invention of FIGS. 1-5.

Optionally, in any embodiment the pH protective coating or layer 286, in the absence of the medicament, has a non-oily appearance. This appearance has been observed in some instances to distinguish an effective pH protective coating or layer from a lubricity layer, which in some instances has been observed to have an oily (i.e. shiny) appearance.

Optionally, for the pH protective coating or layer 286 in any embodiment, the silicon dissolution rate by a 50 mM potassium phosphate buffer diluted in water for injection, adjusted to pH 8 with concentrated nitric acid, and containing 0.2 wt. % polysorbate-80 surfactant, (measured in the absence of the medicament, to avoid changing the dissolution reagent), at 40° C., is less than 170 ppb/day. (Polysorbate-80 is a common ingredient of pharmaceutical preparations, available for example as Tween®-80 from Uniqema Americas LLC, Wilmington Delaware.)

Optionally, for containers up to 10 mL, the pH protective coating or layer in any embodiment, the silicon dissolution rate is less than 160 ppb/day, or less than 140 ppb/day, or less than 120 ppb/day, or less than 100 ppb/day, or less than 90 ppb/day, or less than 80 ppb/day. Optionally, the silicon dissolution rate is more than 10 ppb/day, or more than 20 ppb/day, or more than 30 ppb/day, or more than 40 ppb/day, or more than 50 ppb/day, or more than 60 ppb/day. Any minimum rate stated here can be combined with any maximum rate stated here for the pH protective coating or layer 286 in any embodiment.

Optionally, for the pH protective coating or layer in any embodiment the total silicon content of the pH protective coating or layer and barrier coating, upon dissolution into a test composition with a pH of 8 from the vessel, is less than 66 ppm, or less than 60 ppm, or less than 50 ppm, or less than 40 ppm, or less than 30 ppm, or less than 20 ppm.

The pH protective coating or layer has an interior surface facing the lumen 212 and an outer surface facing the interior surface of the barrier coating or layer 288. Optionally, the pH protective coating or layer is at least coextensive with the barrier coating or layer 288. The pH protective coating or layer alternatively can be less extensive than the barrier coating, as when the fluid does not contact or seldom is in contact with certain parts of the barrier coating absent the pH protective coating or layer. The pH protective coating or layer 286 alternatively can be more extensive than the barrier coating, as it can cover areas that are not provided with a barrier coating.

The pH protective coating or layer 286 optionally can be applied by plasma enhanced chemical vapor deposition (PECVD) of a precursor feed comprising an acyclic siloxane, a monocyclic siloxane, a polycyclic siloxane, a polysilsesquioxane, a monocyclic silazane, a polycyclic silazane, a polysilsesquiazane, a silatrane, a silquasilatrane, a silproatrane, an azasilatrane, an azasilquasiatrane, an azasilproatrane, or a combination of any two or more of these precursors. Some particular, non-limiting precursors contemplated for such use include octamethylcyclotetrasiloxane (OMCTS).

Optionally, an FTIR absorbance spectrum of the pH protective coating or layer 286 has a ratio greater than 0.75 between the maximum amplitude of the Si—O—Si symmetrical stretch peak between about 1000 and 1040 cm⁻¹, and the maximum amplitude of the Si—O—Si assymetric stretch peak between about 1060 and about 1100 cm⁻¹.

In the presence of a fluid composition having a pH between 5 and 9 contained in the lumen, the calculated shelf life of the vessel is up to 36 months at a storage temperature of 4° C. Optionally, the rate of erosion of the pH protective coating or layer 286, if directly contacted by a fluid composition having a pH of 8, is less than 20% optionally less than 15%, optionally less than 10%, optionally less than 7%, optionally from 5% to 20%, optionally 5% to 15%, optionally 5% to 10%, optionally 5% to 7%, of the rate of erosion of the barrier coating or layer, if directly contacted by the same fluid composition under the same conditions. Optionally, the fluid composition removes the pH protective coating or layer 286 at a rate of 1 nm or less of pH protective coating or layer thickness per 44 hours of contact with the fluid composition.

Optionally, the silicon dissolution rate of the pH protective coating or layer and barrier coating or layer by a 50 mM potassium phosphate buffer diluted in water for injection, adjusted to pH 8 with concentrated nitric acid, and containing 0.2 wt. % polysorbate-80 surfactant from the vessel is less than 170 parts per billion (ppb)/day for containers up to 10 mL.

Optionally, the total silicon content of the pH protective coating or layer 286 and the barrier coating or layer 288, upon dissolution into 0.1 N potassium hydroxide aqueous solution at 40° C. from the vessel, is less than 66 ppm for containers up to 10 mL.

Optionally, the calculated shelf life of the vessel 210 (total Si/Si dissolution rate) is more than 2 years.

Optionally, the pH protective coating or layer 286 shows an O-Parameter measured with attenuated total reflection (ATR) of less than 0.4, measured as:

$$O - Parameter = \frac{Intensity at 1253 {cm}^{- 1}}{Maximum intensity in the range 1000 to 1100 {cm}^{- 1} .}$$

The O-Parameter is defined in U.S. Pat. No. 8,067,070, which claims an O-parameter value of most broadly from 0.4 to 0.9. It can be measured from physical analysis of an FTIR amplitude versus wave number plot to find the numerator and denominator of the above expression, as shown in FIG. 5 of U.S. Pat. No. 8,067,070, except annotated to show interpolation of the wave number and absorbance scales to arrive at an absorbance at 1253 cm-1 of 0.0424 and a maximum absorbance at 1000 to 1100 cm-1 of 0.08, resulting in a calculated O-parameter of 0.53. The O-Parameter can also be measured from digital wave number versus absorbance data.

U.S. Pat. No. 8,067,070 asserts that the claimed O-parameter range provides a superior pH protective coating or layer, relying on experiments only with HMDSO and HMDSN, which are both non-cyclic siloxanes. Surprisingly, it has been found by the present inventors that O-parameters outside the ranges claimed in U.S. Pat. No. 8,067,070 provide even better results than are obtained in U.S. Pat. No. 8,067,070. Alternatively in the embodiment of FIGS. 1-5, the O-parameter has a value of from 0.1 to 0.39, or from 0.15 to 0.37, or from 0.17 to 0.35.

Optionally, the pH protective coating or layer shows an N-Parameter measured with attenuated total reflection (ATR) of less than 0.7, measured as:

$$N - Parameter = \frac{Intensity at 840 {cm}^{- 1}}{Intensity at 799 {cm}^{- 1} .}$$

The N-Parameter is also described in U.S. Pat. No. 8,067,070, and is measured analogously to the O-Parameter except that intensities at two specific wave numbers are used—neither of these wave numbers is a range. U.S. Pat. No. 8,067,070 claims a passivation layer with an N-Parameter of 0.7 to 1.6. Again, the present inventors have made better coatings employing a pH protective coating or layer 286 having an N-Parameter lower than 0.7, as described above. Alternatively, the N-parameter has a value of at least 0.3, or from 0.4 to 0.6, or at least 0.53.

The protective coating or layer of Si_{w}OₓC_{y}H_{z} or its equivalent SiOₓC_{y} also can have utility as a hydrophobic layer, independent of whether it also functions as a pH protective coating or layer. Suitable hydrophobic coatings or layers and their application, properties, and use are described in U.S. Pat. No. 7,985,188. Dual functional protective/hydrophobic coatings or layers having the properties of both types of coatings or layers can be provided for any embodiment of the present invention.

### Graded Composite Layer

Another expedient contemplated here, for adjacent layers of SiOₓ and a pH protective coating or layer, is a graded composite of any two or more adjacent PECVD layers, for example the barrier coating or layer 288 and a pH protective coating or layer 286 and/or a lubricity coating or layer 281. A graded composite can be separate layers of a protective and/or barrier layer or coating with a transition or interface of intermediate composition between them, or separate layers of a protective and/or hydrophobic layer and SiOₓ with an intermediate distinct pH protective coating or layer of intermediate composition between them, or a single coating or layer that changes continuously or in steps from a composition of a protective and/or hydrophobic layer to a composition more like SiOₓ, going through the primer coating or layer in a normal direction.

The grade in the graded composite can go in either direction. For example, the composition of SiOₓ can be applied directly to the substrate and graduate to a composition further from the surface of a primer coating or layer, and optionally can further graduate to another type of coating or layer, such as a hydrophobic coating or layer or a lubricity coating or layer. Additionally, in any embodiment an adhesion coating or layer, for example Si_{w}OₓC_{y} or its equivalent SiOₓC_{y}H_{z}, optionally can be applied directly to the substrate before applying the barrier layer. A graduated primer coating or layer is particularly contemplated if a layer of one composition is better for adhering to the substrate than another, in which case the better-adhering composition can, for example, be applied directly to the substrate. It is contemplated that the more distant portions of the graded primer coating or layer can be less compatible with the substrate than the adjacent portions of the graded primer coating or layer, since at any point the primer coating or layer is changing gradually in properties, so adjacent portions at nearly the same depth of the primer coating or layer have nearly identical composition, and more widely physically separated portions at substantially different depths can have more diverse properties. It is also contemplated that a primer coating or layer portion that forms a better barrier against transfer of material to or from the substrate can be directly against the substrate, to prevent the more remote primer coating or layer portion that forms a poorer barrier from being contaminated with the material intended to be barred or impeded by the barrier.

The applied coatings or layers, instead of being graded, optionally can have sharp transitions between one layer and the next, without a substantial gradient of composition. Such primer coating or layer can be made, for example, by providing the gases to produce a layer as a steady state flow in a non-plasma state, then energizing the system with a brief plasma discharge to form a coating or layer on the substrate. If a subsequent primer coating or layer is to be applied, the gases for the previous primer coating or layer are cleared out and the gases for the next primer coating or layer are applied in a steady-state fashion before energizing the plasma and again forming a distinct layer on the surface of the substrate or its outermost previous primer coating or layer, with little if any gradual transition at the interface.

An embodiment can be carried out under conditions effective to form a hydrophobic pH protective coating or layer on the substrate. Optionally, the hydrophobic characteristics of the pH protective coating or layer can be set by setting the ratio of the O₂ to the organosilicon precursor in the gaseous reactant, and/or by setting the electric power used for generating the plasma. Optionally, the pH protective coating or layer can have a lower wetting tension than the uncoated surface, optionally a wetting tension of from 20 to 72 dyne/cm, optionally from 30 to 60 dynes/cm, optionally from 30 to 40 dynes/cm, optionally 34 dyne/cm. Optionally, the pH protective coating or layer can be more hydrophobic than the uncoated surface.

### Equipment

### PECVD Apparatus for Forming PECVD Coating or Layer

PECVD apparatus, a system and precursor materials suitable for applying any of the PECVD coatings or layers described in this specification, specifically including the tie coating or layer 289, the barrier coating or layer 288, or the pH protective coating or layer 286 is described in described in U.S. Pat. No. 7,985,188, which is incorporated by reference.

The vessels having walls 214 can be conveyed to a tie coater 302, which is suitable apparatus for applying a tie coating or layer to the interior surface of the wall, such as the PECVD apparatus described in U.S. Pat. No. 7,985,188.

The vessels can then be conveyed to a barrier coater 304, which is suitable apparatus for applying a barrier coating or layer to the interior surface of the wall, such as the PECVD apparatus described in U.S. Pat. No. 7,985,188.

The vessels can then be conveyed to a pH protective coater 306, which is suitable apparatus for applying a pH protective coating or layer to the interior surface of the wall, such as the PECVD apparatus described in U.S. Pat. No. 7,985,188. This then completes the coating set.

Optionally, further steps can be carried out by the system. For example, the coated vessels can be conveyed to a fluid filler 308 which places fluid from a fluid supply 310 into the lumens of the coated vessels.

For another example the filled vessels can be conveyed to a closure installer 312, which takes closures, for example plungers or stoppers, from a closure supply 314 and seats them in the lumens of the coated vessels.

In any embodiment of the invention, the tie coating or layer optionally can be applied by plasma enhanced chemical vapor deposition (PECVD).

In any embodiment of the invention, the barrier coating or layer optionally can be applied by PECVD.

In any embodiment of the invention, the pH protective coating or layer optionally can be applied by PECVD.

In any embodiment of the invention, the vessel can comprise or consist of a syringe barrel, a vial, cartridge or a blister package.

Reaction conditions for forming the SiOₓ barrier layer are described in U.S. Pat. No. 7,985,188, which is incorporated by reference.

The tie or adhesion coating or layer can be produced, for example, using as the precursor tetramethyldisiloxane (TMDSO) or hexamethyldisiloxane (HMDSO) at a flow rate of 0.5 to 10 sccm, preferably 1 to 5 sccm; oxygen flow of 0.25 to 5 sccm, preferably 0.5 to 2.5 sccm; and argon flow of 1 to 120 sccm, preferably in the upper part of this range for a 1 mL syringe and the lower part of this range for a 5 ml. vial. The overall pressure in the vessel during PECVD can be from 0.01 to 10 Torr, preferably from 0.1 to 1.5 Torr. The power level applied can be from 5 to 100 Watts, preferably in the upper part of this range for a 1 mL syringe and the lower part of this range for a 5 ml. vial. The deposition time (i.e. “on” time for RF power) is from 0.1 to 10 seconds, preferably 1 to 3 seconds. The power cycle optionally can be ramped or steadily increased from 0 Watts to full power over a short time period, such as 2 seconds, when the power is turned on, which may improve the plasma uniformity. The ramp up of power over a period of time is optional, however.

The pH protective coating or layer 286 coating or layer described in this specification can be applied in many different ways. For one example, the low-pressure PECVD process described in U.S. Pat. No. 7,985,188 can be used. For another example, instead of using low-pressure PECVD, atmospheric PECVD can be employed to deposit the pH protective coating or layer. For another example, the coating can be simply evaporated and allowed to deposit on the SiOₓ layer to be protected. For another example, the coating can be sputtered on the SiOₓ layer to be protected. For still another example, the pH protective coating or layer 286 can be applied from a liquid medium used to rinse or wash the SiOₓ layer.

Other precursors and methods can be used to apply the pH protective coating or layer or passivating treatment. For example, hexamethylene disilazane (HMDZ) can be used as the precursor. HMDZ has the advantage of containing no oxygen in its molecular structure. This passivation treatment is contemplated to be a surface treatment of the SiOₓ barrier layer with HMDZ. To slow down and/or eliminate the decomposition of the silicon dioxide coatings at silanol bonding sites, the coating must be passivated. It is contemplated that passivation of the surface with HMDZ (and optionally application of a few mono layers of the HMDZ-derived coating) will result in a toughening of the surface against dissolution, resulting in reduced decomposition. It is contemplated that HMDZ will react with the —OH sites that are present in the silicon dioxide coating, resulting in the evolution of NH3 and bonding of S—(CH3)3 to the silicon (it is contemplated that hydrogen atoms will be evolved and bond with nitrogen from the HMDZ to produce NH3).

It is contemplated that this HMDZ passivation can be accomplished through several possible paths.

One contemplated path is dehydration/vaporization of the HMDZ at ambient temperature. First, an SiOₓ surface is deposited, for example using hexamethylene disiloxane (HMDSO). The as-coated silicon dioxide surface is then reacted with HMDZ vapor. In an embodiment, as soon as the SiOₓ surface is deposited onto the article of interest, the vacuum is maintained. The HMDSO and oxygen are pumped away and a base vacuum is achieved. Once base vacuum is achieved, HMDZ vapor is flowed over the surface of the silicon dioxide (as coated on the part of interest) at pressures from the mTorr range to many Torr. The HMDZ is then pumped away (with the resulting NH₃ that is a by-product of the reaction). The amount of NH3 in the gas stream can be monitored (with a residual gas analyzer—RGA—as an example) and when there is no more NH3 detected, the reaction is complete. The part is then vented to atmosphere (with a clean dry gas or nitrogen). The resulting surface is then found to have been passivated. It is contemplated that this method optionally can be accomplished without forming a plasma.

Alternatively, after formation of the SiOₓ barrier coating or layer, the vacuum can be broken before dehydration/vaporization of the HMDZ. Dehydration/vaporization of the HMDZ can then be carried out in either the same apparatus used for formation of the SiOₓ barrier coating or layer or different apparatus.

Dehydration/vaporization of HMDZ at an elevated temperature is also contemplated. The above process can alternatively be carried out at an elevated temperature exceeding room temperature up to about 150° C. The maximum temperature is determined by the material from which the coated part is constructed. An upper temperature should be selected that will not distort or otherwise damage the part being coated.

Dehydration/vaporization of HMDZ with a plasma assist is also contemplated. After carrying out any of the above embodiments of dehydration/vaporization, once the HMDZ vapor is admitted into the part, a plasma is generated. The plasma power can range from a few watts to 100+ watts (similar powers as used to deposit the SiOₓ). The above is not limited to HMDZ and could be applicable to any molecule that will react with hydrogen, for example any of the nitrogen-containing precursors described in this specification.

Another way of applying the pH protective coating or layer is to apply as the pH protective coating or layer an amorphous carbon or fluorocarbon coating, or a combination of the two.

Amorphous carbon coatings can be formed by PECVD using a saturated hydrocarbon, (e.g. methane or propane) or an unsaturated hydrocarbon (e.g. ethylene, acetylene) as a precursor for plasma polymerization. Fluorocarbon coatings can be derived from fluorocarbons (for example, hexafluoroethylene or tetrafluoroethylene). Either type of coating, or a combination of both, can be deposited by vacuum PECVD or atmospheric pressure PECVD. It is contemplated that that an amorphous carbon and/or fluorocarbon coating will provide better passivation of an SiOₓ barrier layer than a siloxane coating since an amorphous carbon and/or fluorocarbon coating will not contain silanol bonds.

It is further contemplated that fluorosilicon precursors can be used to provide a pH protective coating or layer over an SiOₓ barrier layer. This can be carried out by using as a precursor a fluorinated silane precursor such as hexafluorosilane and a PECVD process. The resulting coating would also be expected to be a non-wetting coating.

It is further contemplated that any embodiment of the pH protective coating or layer processes described in this specification can also be carried out without using the article to be coated to contain the plasma. For example, external surfaces of medical articles, for example catheters, surgical instruments, closures, and others can be protected or passivated by sputtering the coating, employing a radio frequency target.

Yet another coating modality contemplated for protecting or passivating an SiOₓ barrier layer is coating the barrier layer using a polyamidoamine epichlorohydrin resin. For example, the barrier coated part can be dip coated in a fluid polyamidoamine epichlorohydrin resin melt, solution or dispersion and cured by autoclaving or other heating at a temperature between 6° and 100° C. It is contemplated that a coating of polyamidoamine epichlorohydrin resin can be preferentially used in aqueous environments between pH 5-8, as such resins are known to provide high wet strength in paper in that pH range. Wet strength is the ability to maintain mechanical strength of paper subjected to complete water soaking for extended periods of time, so it is contemplated that a coating of polyamidoamine epichlorohydrin resin on an SiOₓ barrier layer will have similar resistance to dissolution in aqueous media. It is also contemplated that, because polyamidoamine epichlorohydrin resin imparts a lubricity improvement to paper, it will also provide lubricity in the form of a coating on a thermoplastic surface made of, for example, COC or COP.

Even another approach for protecting an SiOₓ layer is to apply as a pH protective coating or layer a liquid-applied coating of a polyfluoroalkyl ether, followed by atmospheric plasma curing the pH protective coating or layer. For example, it is contemplated that the process practiced under the trademark TriboGlide®, described in this specification, can be used to provide a pH protective coating or layer that is also a lubricity layer, as TriboGlide® is conventionally used to provide lubricity.

Exemplary PECVD reaction conditions for preparing a pH protective coating or layer 286 in a 3 ml sample size syringe with a ⅛″ diameter tube (open at the end) are as follows:

For depositing a pH protective coating or layer, a precursor feed or process gas can be employed having a standard volume ratio of, for example:
from 0.5 to 10 standard volumes, optionally from 1 to 6 standard volumes, optionally from 2 to 4 standard volumes, optionally equal to or less than 6 standard volumes, optionally equal to or less than 2.5 standard volumes, optionally equal to or less than 1.5 standard volumes, optionally equal to or less than 1.25 standard volumes of the precursor, for example OMCTS or one of the other precursors of any embodiment;
from 0 to 100 standard volumes, optionally from 1 to 200 standard volumes, optionally from 1 to 80 standard volumes, optionally from 5 to 100 standard volumes, optionally from 10 to 70 standard volumes, of a carrier gas of any embodiment, for example argon.
from 0.1 to 10 standard volumes, optionally from 0.1 to 2 standard volumes, optionally from 0.2 to 1.5 standard volumes, optionally from 0.2 to 1 standard volumes, optionally from 0.5 to 1.5 standard volumes, optionally from 0.8 to 1.2 standard volumes of an oxidizing agent.
The power level can be, for example, from 0.1-500 watts.
Specific Flow rates and power levels contemplated include:
   OMCTS: 2.0 sccm
   Oxygen: 0.7 sccm
   Argon: 7.0 sccm
   Power: 3.5 watts

### Surface Treatments

“Plasma,” as referenced in any embodiment, has its conventional meaning in physics of one of the four fundamental states of matter, characterized by extensive ionization of its constituent particles, a generally gaseous form, and incandescence (i.e. it produces a glow discharge, meaning that it emits light).

“Conversion plasma treatment” refers to any plasma treatment that reduces the adhesion of one or more biomolecules to a treated surface.

“Conditioning plasma treatment” refers to any plasma treatment of a surface to prepare the surface for further conversion plasma treatment. “Conditioning plasma treatment” includes a plasma treatment that, in itself, reduces the adhesion of one or more biomolecules to a treated surface, but is followed by conversion plasma treatment that further reduces the adhesion of one or more biomolecules to a treated surface. “Conditioning plasma treatment” also includes a plasma treatment that, in itself, does not reduce the adhesion of one or more biomolecules to a treated surface.

A “remote” conversion plasma treatment, generally speaking, is conversion plasma treatment of a surface located at a “remote” point where the radiant energy density of the plasma, for example in Joules per cm3, is substantially less than the maximum radiant energy density at any point of the plasma glow discharge (referred to below as the “brightest point”), but the remote surface is close enough to some part of the glow discharge to reduce the adhesion of one or more biomolecules to the treated remote surface. “Remote” is defined in the same manner respecting a remote conditioning plasma treatment, except that the remote surface must be close enough to some part of the glow discharge to condition the surface.

The radiant energy density at the brightest point of the plasma is determined spectrophotometrically by measuring the radiant intensity of the most intense emission line of light in the visible spectrum (380 nanometer (nm) to 750 nm wavelength) at the brightest point. The radiant energy density at the remote point is determined spectrophotometrically by measuring the radiant energy density of the same emission line of light at the remote point. “Remoteness” of a point is quantified by measuring the ratio of the radiant energy density at the remote point to the radiant energy density at the brightest point. The present specification and claims define “remote” quantitatively as a specific range of that ratio. Broadly, the ratio is from 0 to 0.5, optionally from 0 to 0.25, optionally about 0, optionally exactly 0. Remote conversion plasma treatment can be carried out where the ratio is zero, even though that indicates no measurable visible light at the remote point, because the dark discharge region or afterglow region of plasma contain energetic species that, although not energetic enough to emit light, are energetic enough to modify the treated surface to reduce the adhesion of one or more biomolecules.

A “non-polymerizing compound” is defined operationally for all embodiments as a compound that does not polymerize on a treated surface or otherwise form an additive coating under the conditions used in a particular plasma treatment of the surface. Numerous, non-limiting examples of compounds that can be used under non-polymerizing conditions are the following: O₂, N₂, air, O₃, N₂O, H₂, H₂O₂, NH₃, Ar, He, Ne, and combinations of any of two or more of the foregoing. These may also include alcohols, organic acids, and polar organic solvents as well as materials that can be polymerized under different plasma conditions from those employed. “Non-polymerizing” includes compounds that react with and bond to a pre-existing polymeric surface and locally modify its composition at the surface. The essential characterizing feature of a non-polymerizing coating is that it does not build up thickness (i.e. build up an additive coating) as the treatment time is increased.

A “substrate” is an article or other solid form (such as a granule, bead, or particle).

A “surface” is broadly defined as either an original surface (a “surface” also includes a portion of a surface wherever used in this specification) of a substrate, or a coated or treated surface prepared by any suitable coating or treating method, such as liquid application, condensation from a gas, or chemical vapor deposition, including plasma enhanced chemical vapor deposition carried out under conditions effective to form a coating on the substrate.

A treated surface is defined for all embodiments as a surface that has been plasma treated as described in this specification.

The terms “optionally” and “alternatively” are regarded as having the same meaning in the present specification and claims, and may be used interchangeably.

The “material” in any embodiment can be any material of which a substrate is formed, including but not limited to a thermoplastic material, optionally a thermoplastic injection moldable material. The substrate according to any embodiment may be made, for example, from material including, but not limited to: an olefin polymer; polypropylene (PP); polyethylene (PE); cyclic olefin copolymer (COC); cyclic olefin polymer (COP); polymethylpentene; polyester; polyethylene terephthalate; polyethylene naphthalate; polybutylene terephthalate (PBT); PVdC (polyvinylidene chloride); polyvinyl chloride (PVC); polycarbonate; polymethylmethacrylate; polylactic acid; polystyrene; hydrogenated polystyrene; poly(cyclohexylethylene) (PCHE); epoxy resin; nylon; polyurethane polyacrylonitrile; polyacrylonitrile (PAN); an ionomeric resin; or Surlyn® ionomeric resin.

The term “vessel” as used throughout this specification may be any type of article that is adapted to contain or convey a liquid, a gas, a solid, or any two or more of these. One example of a vessel is an article with at least one opening (e.g., one, two or more, depending on the application) and a wall defining an interior contacting surface.

The term “stress conditions” can be of any form, such as acidic or basic condition, agitation, movement, freeze-thaw cycles, storing at extended period of time, etc.

The present method for treating a surface, optionally a surface of a substrate, includes treating the surface with conversion plasma of one or more non-polymerizing compounds, in a chamber, to form a treated surface.

A wide variety of different surfaces can be treated according to any embodiment. One example of a surface is a vessel lumen surface, where the vessel is, for example, a vial, a bottle, a jar, a syringe, a cartridge, a blister package, or an ampoule. For more examples, the surface of the material can be a fluid surface of an article of labware, for example a microplate, a centrifuge tube, a pipette tip, a well plate, a microwell plate, an ELISA plate, a microtiter plate, a 96-well plate, a 384-well plate, a centrifuge tube, a chromatography vial, an evacuated blood collection tube, or a specimen tube.

The treated surface of any embodiment can be a coating or layer of PECVD deposited SiOₓC_{y}H_{z} or SiNxCyHz, in which x is from about 0.5 to about 2.4 as measured by X-ray photoelectron spectroscopy (XPS), y is from about 0.6 to about 3 as measured by XPS, and z is from about 2 to about 9, optionally from about 2 to about 6, as measured by Rutherford backscattering spectrometry (RBS). Another example of the surface to be treated is a barrier coating or layer of SiOₓ, in which x is from about 1.5 to about 2.9 as measured by XPS, optionally an oxide or nitride of an organometallic precursor that is a compound of a metal element from Group III and/or Group IV of the Periodic Table, e.g. in Group III: Boron, Aluminum, Gallium, Indium, Thallium, Scandium, Yttrium, or Lanthanum, (Aluminum and Boron being preferred), and in Group TV: Silicon, Germanium, Tin, Lead, Titanium, Zirconium, Hafnium, or Thorium (Silicon and Tin being preferred).

The gas or gases employed to treat the surface in any embodiment can be an inert gas or a reactive gas, and can be any of the following: O₂, N₂, air, O₃, N₂O, NO₂, N₂O₄, H₂, H₂O₂, H₂O, NH₃, Ar, He, Ne, Xe, Kr, a nitrogen-containing gas, other non-polymerizing gases, gas combinations including an Ar/O₂ mix, an N₂/O₂ mix following a pre-treatment conditioning step with Ar, a volatile and polar organic compound, the combination of a C₁-C₁₂ hydrocarbon and oxygen; the combination of a C₁-C₁₂ hydrocarbon and nitrogen; a silicon-containing gas; or a combination of two or more of these. The treatment employs a non-polymerizing gas as defined in this specification.

The volatile and polar organic compound of any embodiment can be, for example water, for example tap water, distilled water, or deionized water; an alcohol, for example a C₁-C₁₂ alcohol, methanol, ethanol, n-propanol, isopropanol, n-butanol, isobutanol, s-butanol, t-butanol; a glycol, for example ethylene glycol, propylene glycol, butylene glycol, polyethylene glycol, and others; glycerine, a C₁-C₁₂ linear or cyclic ether, for example dimethyl ether, diethyl ether, dipropyl ether, dibutyl ether, glyme (CH₃OCH₂CH₂OCH₃); cyclic ethers of formula —CH₂CH₂O— such as diethylene oxide, triethylene oxide, and tetraethylene oxide; cyclic amines; cyclic esters (lactones), for example acetolactone, propiolactone, butyrolactone, valerolactone, and caprolactone; a C₁-C₁₂ aldehyde, for example formaldehyde, acetaldehyde, propionaldehyde, or butyraldehyde; a C₁-C₁₂ ketone, for example acetone, diethylketone, dipropylketone, or dibutylketone; a C₁-C₁₂ carboxylic acid, for example formic acid, acetic acid, propionic acid, or butyric acid; ammonia, a C₁-C₁₂ amine, for example methylamine, dimethylamine, ethylamine, diethylamine, propylamine, butylamine, pentylamine, hexylamine, heptylamine, octylamine, nonylamine, decylamine, undecylamine, or dodecylamine; hydrogen fluoride, hydrogen chloride, a C₁-C₁₂ epoxide, for example ethylene oxide or propylene oxide; or a combination of any two or more of these.

The C₁-C₁₂ hydrocarbon of any embodiment optionally can be methane, ethane, ethylene, acetylene, n-propane, i-propane, propene, propyne; n-butane, i-butane, t-butane, butane, 1-butyne, 2-butyne, or a combination of any two or more of these.

The silicon-containing gas of any embodiment can be a silane, an organosilicon precursor, or a combination of any two or more of these. The silicon-containing gas can be an acyclic or cyclic, substituted or unsubstituted silane, optionally comprising, consisting essentially of, or consisting of any one or more of: Si₁-Si₄ substituted or unsubstituted silanes, for example silane, disilane, trisilane, or tetrasilane; hydrocarbon or halogen substituted Si₁-Si₄ silanes, for example tetramethylsilane (TetraMS), tetraethyl silane, tetrapropylsilane, tetrabutylsilane, trimethylsilane (TriMS), triethyl silane, tripropylsilane, tributylsilane, trimethoxysilane, a fluorinated silane such as hexafluorodisilane, a cyclic silane such as octamethylcyclotetrasilane or tetramethylcyclotetrasilane, or a combination of any two or more of these. The silicon-containing gas can be a linear siloxane, a monocyclic siloxane, a polycyclic siloxane, a polysilsesquioxane, an alkyl trimethoxysilane, a linear silazane, a monocyclic silazane, a polycyclic silazane, a polysilsesquiazane, or a combination of any two or more of these, for example hexamethyldisiloxane (HMDSO), tetramethyldisiloxane (TMDSO), octamethylcyclotetrasiloxane (OMCTS), tetramethyldisilazane, hexamethyldisilazane, octamethyltrisilazane, octamethylcyclotetrasilazane, tetramethylcyclotetrasilazane, or a combination of any two or more of these.

The electrical power used to excite the plasma used in plasma treatment in any embodiment, can be, for example, from 1 to 1000 Watts, optionally from 100 to 900 Watts, optionally from 50 to 600 Watts, optionally 100 to 500 Watts, optionally from 500 to 700 Watts, optionally from 1 to 100 Watts, optionally from 1 to 30 Watts, optionally from 1 to 10 Watts, optionally from 1 to 5 Watts.

The frequency of the electrical power used to excite the plasma used in plasma treatment, in any embodiment, can be any type of energy that will ignite plasma in the plasma zone. For example, it can be direct current (DC) or alternating current (electromagnetic energy) having a frequency from 3 Hz to 300 GHz. Electromagnetic energy in this range generally includes radio frequency (RF) energy and microwave energy, more particularly characterized as extremely low frequency (ELF) of 3 to 30 Hz, super low frequency (SLF) of 30 to 300 Hz, voice or ultra-low frequency (VF or ULF) of 300 Hz to 3 kHz, very low frequency (VLF) of 3 to 30 kHz, low frequency (LF) of 30 to 300 kHz, medium frequency (MF) of 300 kHz to 3 MHz, high frequency (HF) of 3 to 30 MHz, very high frequency (VHF) of 30 to 300 MHz, ultra-high frequency (UHF) of 300 MHz to 3 GHz, super high frequency (SHF) of 3 to 30 GHz, extremely high frequency (EHF) of 30 to 300 GHz, or any combination of two or more of these frequencies. For example, high frequency energy, commonly 13.56 MHz, is useful RF energy, and ultra-high frequency energy, commonly 2.54 GHz, is useful microwave energy, as two non-limiting examples of commonly used frequencies.

The plasma exciting energy, in any embodiment, can either be continuous during a treatment step or pulsed multiple times during the treatment step. If pulsed, it can alternately pulse on for times ranging from one millisecond to one second, and then off for times ranging from one millisecond to one second, in a regular or varying sequence during plasma treatment. One complete duty cycle (one “on” period plus one “off” period) can be 1 to 2000 milliseconds (ms), optionally 1 to 1000 milliseconds (ms), optionally 2 to 500 ms, optionally 5 to 100 ms, optionally 10 to 100 ms long.

Optionally in any embodiment, the relation between the power on and power off portions of the duty cycle can be, for example, power on 1-90 percent of the time, optionally on 1-80 percent of the time, optionally on 1-70 percent of the time, optionally on 1-60 percent of the time, optionally on 1-50 percent of the time, optionally on 1-45 percent of the time, optionally on 1-40 percent of the time, optionally on 1-35 percent of the time, optionally on 1-30 percent of the time, optionally on 1-25 percent of the time, optionally on 1-20 percent of the time, optionally on 1-15 percent of the time, optionally on 1-10 percent of the time, optionally on 1-5 percent of the time, and power off for the remaining time of each duty cycle.

The plasma pulsing described in Mark J. Kushner, Pulsed Plasma-Pulsed Injection Sources For Remote Plasma Activated Chemical Vapor Deposition, J. APPL. PHYS. 73, 4098 (1993), can optionally be used.

The flow rate of process gas during plasma treatment according to any embodiment can be from 1 to 300 sccm (standard cubic centimeters per minute), optionally 1 to 200 sccm, optionally from 1 to 100 sccm, optionally 1-50 sccm, optionally 5-50 sccm, optionally 1-10 sccm.

Optionally in any embodiment, the plasma chamber is reduced to a base pressure from 0.001 milliTorr (mTorr, 0.00013 Pascal) to 100 Torr (13,000 Pascal) before feeding gases. Optionally the feed gas pressure in any embodiment can range from 0.001 to 10,000 mTorr (0.00013 to 1300 Pascal), optionally from 1 mTorr to 10 Torr (0.13 to 1300 Pascal), optionally from 0.001 to 5000 mTorr (0.00013 to 670 Pascal), optionally from 1 to 1000 milliTorr (0.13 to 130 Pascal).

The treatment volume in which the plasma is generated in any embodiment can be, for example, from 100 mL to 50 liters, preferably 8 liters to 20 liters, if a treatment vessel separate from the treated surface is used, and, for example, from 0.1 to 20 mL, optionally from 0.5 to 10 mL if the treated surface is part of the interior surface of a vessel serving also as the vessel in which the plasma is contained, for example if the treatment vessel is a syringe barrel, vial, or cartridge intended to serve as primary packaging for a drug.

The plasma treatment time in any embodiment can be, for example, from 1 to 300 seconds, optionally 3 to 300 sec., optionally 30 to 300 sec., optionally 150 to 250 sec., optionally 150 to 200 sec., optionally from 90 to 180 seconds.

The number of plasma treatment steps can vary, in any embodiment. For example one plasma treatment can be used; optionally two or more plasma treatments can be used, employing the same or different conditions.

In any embodiment, the plasma treatment apparatus employed can be any suitable apparatus. Plasma treatment apparatus of the type that employs the lumen of the vessel to be treated as a vacuum chamber, shown for example in U.S. Pat. No. 7,985,188, FIG. 2, can be used in any embodiment.

The plasma treatment process of any embodiment optionally can be combined with treatment using an ionized gas. The ionized gas can be, as some examples, any of the gases identified as suitable for plasma treatment. The ionized gas can be delivered in any suitable manner. For example, it can be delivered from an ionizing blow-off gun or other ionized gas source. A convenient gas delivery pressure is from 1-120 psi (pounds per square inch) (6 to 830 kPa, kiloPascals) (gauge or, optionally, absolute pressure), optionally 50 psi (350 kPa). The water content of the ionized gas can be from 0 to 100%. The polar-treated surface with ionized gas can be carried out for any suitable treatment time, for example from 1-300 seconds, optionally for 10 seconds.

### I-E Closure

Optionally in any embodiment, the internal surface of the primary drug container is generally cylindrical, further comprising a plunger or stopper positioned in and slidable within the internal surface. Optionally in any embodiment, the plunger is an O-ring plunger.

Optionally in any embodiment, the plunger is a two-position plunger having a first position for use while storing the primary drug container and a second position for use while dispensing a drug from the primary drug container.

Optionally in any embodiment, the plunger is a two-position plunger having a first position for use while storing the primary drug container and a second position for use while dispensing a drug from the primary drug container with lubricant.

Optionally in any embodiment, the plunger is a two-position plunger having a first position for use while storing the primary drug container and a second position for use while dispensing a drug from the primary drug container without applying lubricant to lower the umber of the particles.

Suitable plungers are described, for example, in International Application PCT/US2014/059531, filed 7 Oct. 2014; U.S. Ser. No. 62/192,192, filed Jul. 14, 2015; and U.S. Ser. No. 62/269,600, filed Dec. 18, 2015. The entire text and drawings of these applications are incorporated by reference here in its entirety by reference.

Optionally in any embodiment, the plunger can have a resilient core over which is laminated an external layer of a fluorinated polymer such as polytetrafluoroethylene. Such plungers are available commercially, for example a Datwyler Omniflex plunger composed of a bromobutyl rubber core and a fluoropolymer conformal coating applied to the exterior surface of the plunger to block potential leachates into the drug.

Optionally in any embodiment, the primary drug container includes a hypodermic needle having an internal delivery passage communicating with the lumen and a distal end. Optionally in any embodiment, the primary drug container includes a needle shield. Optionally in any embodiment, the needle distal end is buried in the needle shield.

### Particle Count During Shelf Life

Optionally in any embodiment, the particle count in the primary drug container is measured immediately, optionally one day, optionally one month, optionally three months, optionally one year after water for injection is placed in the lumen.

Optionally in any embodiment, the primary drug container can contain a polypeptide composition in the lumen in contact with the PECVD coating, in which the particle count is measured one day, optionally one month, optionally three months, optionally one year, optionally at the end of the therapeutic shelf life after the polypeptide composition is placed in the lumen.

Micro-flow imaging (MFI) is a particle analysis technique that uses flow microscopy to quantify particles contained in a solution based on size. Optionally in any embodiment, this technique can be used to characterize subvisible particles from approximately 1 μm to >50 μm.

Optionally in any embodiment, Dynamic Image Analysis (DIA) FlowCam® can be used for several measurements. A dynamic imaging particle analyzer performs three functions all in one instrument. The instrument examines a fluid under a microscope, takes images of the magnified particles within that fluid, and characterizes the particles using a variety of measurements. Dynamic imaging particle analysis combines the benefits of manual microscopy with those of volumetric techniques. Microscopic particle measurements are taken from images quickly enough to produce statistically significant results. Additionally, many different measurements are taken for each particle—providing the detailed information often needed for a thorough particle analysis. The addition of specialized software enables sophisticated post-processing of data to give a more in-depth analysis of the sample and a better understanding of the data. The ability of an imaging system to resolve details in a particle is essential for accurate measurement. The optical system and the sensor of the instrument affect its ability to size and characterize sub-visible particles. Because of this, counting in a dynamic imaging particle analysis system should be limited to particles having an equivalent spherical diameter (or ESD) of 1 μm and greater, and particle characterization (i.e. shape) should be limited to particles having an ESD of 2 μm and greater. (The ESD of an irregularly shaped object is defined for the purpose of this application as the diameter of a sphere of equivalent volume.) It is important to optimize the settings on this type of instrument specifically for the sample being analyzed to ensure accurate results. Multiple filters can be created, saved, and reused, which allow the experimenter to separate a sample into its component parts based upon particle properties. When analyzing protein therapeutics this is especially helpful when separating silicone oil from proteins.
Source Fluid Imaging Technologies
   a. Capabilities: count, size, limited morphology
   b. Range: 2-10,000 μm
   c. Concentration: 10⁶ particles/mL

Note: The presence of air, immiscible oil, semi-solids, refractive index variations and extremes of density will yield different results across the three techniques, LO, MM and DIA (Flow Cam).

### WORKING EXAMPLES

### Light Obscuration Particle Count Protocol for Example 1

The injection molded COP test articles used for this testing were 2 mL, 5 mL, 6 mL, 10 mL, or 25 mL vials with a trilayer coating made as described in Example 3 below. For example, 6 mL COP vials were provided with a trilayer coating under the following conditions:

| Gas | MW | units | | Units | Adhesion | Barrier | Protective |
|---|---|---|---|---|---|---|---|
| HMDSO | 162.38 | g/mole | Power | W | 100 | 218 | 108 |
| TMDSO | 134.32 | g/mole | O2 Flow | sccm | 2 | 50 | 2 |
| O2 | 16 | g/mole | HMDSO Flow | sccm | 4 | 1 | 0 |
| Ar | 40 | g/mole | TMDSO Flow | sccm | 0 | 0 | 4 |
| | | | Argon Flow | sccm | 80 | 0 | 80 |
| | | | W/FM | KJ/kg | 41460 | 303539 | 38395 |
| | | | W/FM | J/kg | 4.15E+07 | 3.04E+08 | 3.84E+07 |

The sub-visible particle counts determined in Example 1 below were measured generally in accordance with The United States Pharmacopeia (USP), Chapter 788 (Particulate Matter in Injections), Method 1 (Light Obscuration Particle Count).

The medium for collecting particles was Particle Free Water (PFW). PFW is Type I water (ultra-pure, with a resistivity of 18.2 MΩ) that has been filtered through a 0.22 μm pore-size filter, such as can be obtained from a Millipore MilliQ or equivalent water filtration system.

Particle counting was carried out using a Beckman Coulter HIAC 9703+ Liquid Particle Counter, although one could substitute an equivalent instrument qualified to run USP 788.

### Example 1—Light Obscuration Analysis

A 50 mL polypropylene (PP) sample tube was cleaned by rinsing the outside of the tube with PFW and placing it in a laminar flow hood to dry. The inside of the 50 mL tube was rinsed by adding approximately 5 ml of PFW, capping it, vigorously shaking it, and discarding the PFW, shaking out all drops. This PFW rinse was repeated two more times. The clean PP tube was filled with enough PFW to fill all test articles in a batch and perform the necessary validation testing.

A stopper for the test article was prepared by scrubbing it with soap and water, then thoroughly rinsing with PFW, followed by an isopropyl alcohol (IPA) rinse. The stopper was air dried in the laminar flow hood.

A 5 mL pipette tip was cleaned by rinsing the outside with PFW, then internally rinsed by aspirating PFW to the second stop and discarding the rinse PFW. The internal rinse was repeated two more times.

A blank sample was drawn from the PFW in the PP sample tube and tested for particle count to assure that the blank particle count was about 1 particle/mL in the cumulative count column of the 10 μm channel.

Each test article was filled with PFW, using the pipette tip, according to the fill volumes for various sized parenterals suggested in the table below:

| | | |
|---|---|---|
| Container size | Vol. of Fill | No. to Pool |

| | | | | |
|---|---|---|---|---|
| 2 | ml | 2.5 | ml | 1 |
| 5 | ml | 5.0 | ml | 1 |
| 6 | ml | 6.0 | ml | 1 |
| 10 | ml | 10.0 | ml | 1 |
| 25 | ml | 25.0 | ml | 1 |

The test article was sealed with the prepared stopper. Before stoppering, the stopper was rinsed three more times with PFW.

The particle sample was collected by slowly inverting the test article 20 times to suspend the particles. The stopper was removed from the test article, then its contents and those of the other samples of a batch were combined in a new PP sample tube. The sample tube was allowed to stand 2 minutes before testing to degas. The sample aspiration probe was placed in the sample tube such that the end was very close to the bottom of the sample tube without touching. Then a sample was drawn and tested in the particle counter, using the appropriate USP 788 method in the system software.

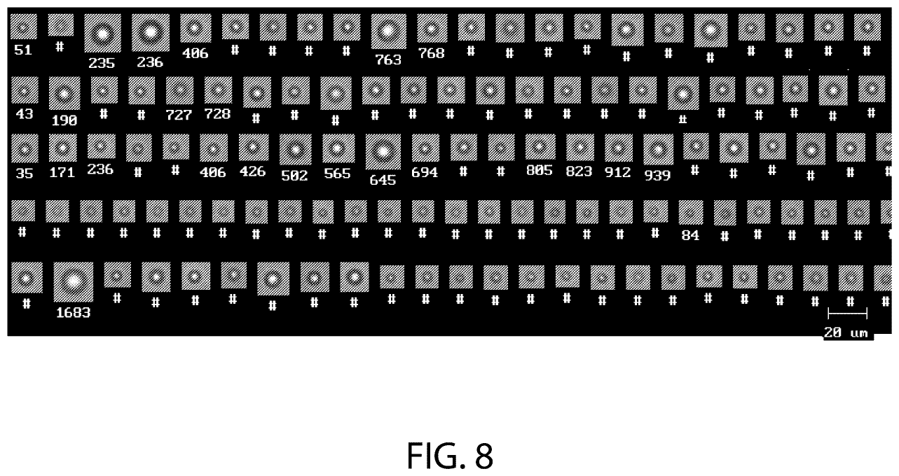

FIGS. 1-3 and 7-8 show the results of testing using the above protocol.

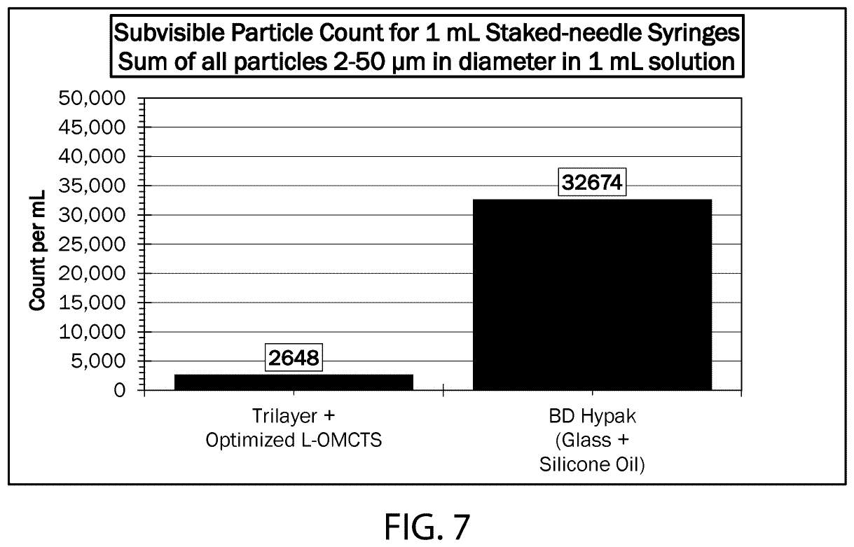

The counts of particles of the indicated sizes measured using the above protocol are for a syringe as described in FIG. 6 with a trilayer coating, further including the optional silicone oil free lubricant (“L-OMCTS”), showing results similar to the left plot in FIG. 7.

### Example 2—FlowCam® Dynamic Image Analysis (DIA)

FlowCam® is a registered trademark of Fluid Imaging Technologies, Inc., Scarborough, Maine, for a dynamic imaging particle analyzer, also known as a dynamic image analyzer. A FlowCam® dynamic imaging particle analyzer performs three functions all in one instrument. The instrument examines a fluid under a microscope, takes images of the magnified particles within that fluid, and characterizes the particles using a variety of measurements. Dynamic imaging particle analysis combines the benefits of manual microscopy with those of volumetric techniques. Microscopic particle measurements are taken from images quickly enough to produce statistically significant results. Additionally, many different measurements are taken for each particle—providing the detailed information often needed for a thorough particle analysis. The addition of specialized software enables sophisticated post-processing of data to give you a more in-depth analysis of the sample and a better understanding of the data. The ability of an imaging system to resolve details in a particle is essential for accurate measurement. The optical system and the sensor of the instrument affect its ability to size and characterize sub-visible particles. Because of this, counting in a dynamic imaging particle analysis system should be limited to particles having an Equivalent Spherical Diameter (ESD) of 1 μm or greater, and particle characterization (i.e. shape) should be limited to particles having an ESD of 2 μm or greater. (The ESD of a particle is the diameter of a sphere of the same volume as the actual particle, and is particularly useful for characterizing and comparing the sizes of non-spherical particles.) It is important to optimize the settings on this type of instrument specifically for the sample you are analyzing to ensure accurate results. Multiple filters can be created, saved, and reused, which allow the analyst to separate a sample into its component parts based upon particle properties. When analyzing protein therapeutics this is especially helpful when separating silicone oil from proteins.—Source Fluid Imaging Technologies
a. Capabilities: count, size, limited morphology
b. Range: 2-10,000 μm
c. Concentration: 106 particles/mL

This example is to evaluate the particle count and size distribution of the trilayer coated and the quadlayer coated syringes of this invention, as extracted with PFW. Typically filled sealed syringes are subject to one of two preparations:
1) 20 slow inversions {USP 788} or
2) Adapted shaker table method used for consistency. Syringes are oriented horizontally on a shaker table and agitated at 1000 rpm for 10 minutes. This allows for free linear movement of the headspace bubble from flange to hub and sufficient time for teasing out slight variations in shedding of lubricants.

The choice of volume aspirated, flow rate, objective lens and flow cell diameter is study specific and based on whether a syringe lubricant evaluation or extrinsic particle contamination characterization (counts, morphology and species).

After cleaning, set-up, focus and system suitability checks samples are dispensed and pooled into a pre-cleaned container (for pooled samples) or aspirated neat (individually) via a pre-cleaned sampling funnel and syringe pump allowing ˜10 minutes for dissipation of suspended air bubbles (verified by time series plot).

The instrument flow cell and sample funnel is cleaned between injections (aspirations).

Post analysis data sort including size as longest dimension, Estimated Spherical Diameter or (ESD) or Average Basal Diameter (ABD), aspect ratio, edge gradient, frequency, and transparency is applied based on study requirements.

The syringes are tested with the following parameters:

### Procedures for Using FlowCam® 10× in Example 2:

1 mL fill volume
20 slow inversions {USP 788}
40 syringes/pool,
10X objective lens,
100 um flow cell
efficiency factor 24.4%,
Aspirated volume 1 mL,
aspiration time 5 min 48 sec,
Dispensing syringe volumes through the needle
10 minute hold (pooled sample) for dissipation of air
bubbles,
Counting/sizing all particles >2 um by ESD
Frame rate 22 frames/sec
flash duration 21.00 microseconds
Distance to nearest neighbor 1 um;
Close hole 4 iterations

### System Calibration-Particle Size and Total Count Verification in Example 2

Perform calibration at the start of each test day to verify the state of system calibration/system performance and prior to any formal testing of samples. Perform suitability on two different reference standards:

Reference standard A—4× Bead Mix (20, 50, 100 microns) to show that the system can detect particles across a reference range.

Reference standard B—Concentration reference standard to show the system can accurately count the number of particles present in a sample of a “known concentration” of particles. (e.g 10 micron and 50 micron beads at ˜300 particles/mL).

Click once on the “visual spreadsheet” icon. Prime the system with filtered water by selecting the “set-up and focus” tab; connect a beaker containing filtered water to the inlet line and an empty waste beaker on the outlet line. On the “pump” window click “start pump” at a flow rate of ˜1 mL/min. Allow the system to flush for at least 5 minutes.

After 5 minutes click “stop pump” and load the appropriate pre-prepared Reference Standard A sample beaker onto the inlet line. Run the Reference Standard solution in the “set-up and focus” mode to flush the line with the reference solution for approximately 1 minute to prime the inlet line and flow cell with the test solution for the forthcoming analysis. Prime the flow cell prior at the start of all runs for any test solution (standard or sample).

Select “analyze” then “auto image* mode”. Select “stop after 1 mL”.

Click “OK” once, enter in the “reason for change” field the name of the test as “Calibration test” and the test date.

Select “accept”

Enter file name (enter the test reference Calibration Test Reference Standard “A—Bead Size” and the analysis date).

Select “OK”; confirm “OK”.

Repeat Steps 6.1 to 6.4.3 for the pre-prepared solution identified as Reference Standard B (e.g 10 micron and 50 micron beads at ˜300 particles/mL), but enter file name as Reference Standard B.

### Acceptance Criteria for Calibration Test in Example 2

Reference Standard “A—Bead Size”. Click each appropriate particle (bead) size to be measured (20, 50, 100 micron sizes, respectively) and observe the peaks that are highlighted on the Frequency vs. Diameter graph. If the highest peak at each measurement is highlighted, then the instrument measured the bead correctly.

Reference Standard “B—Particle Concentration”. The particles/mL value will be displayed on the Main Window and via the “statistics” menu item in the View Window. The measurement of particles should be within ±10% of the C of A's listed particle concentration (documentation from the manufacturer/supplier).

If the instrument fails Calibration notify laboratory supervision. If adjustments are necessary refer to the FlowCam® system manual for optimization of the Optical path and camera Focal Length.

### Analysis in Example 2:

Prime the system with the filtered water sample by selecting the “set-up and focus” tab in the “Setup” menu; connect a beaker containing filtered water to the inlet line and a empty waste beaker on the outlet line. On the “pump” window click “start pump” at a flow rate of ˜1 mL/min. Allow the system to flush for at least 5 minutes.

Test individual containers by inverting 20 times to suspend particles. Pooled samples with remaining volume should be hand swirled or mechanically swirled prior to testing.

After 5 minutes of priming click “pause pump” and switch the inlet line from filtered water into the test solution. Run the test solution in the “set-up and focus” mode through the inlet line by clicking “resume pump” to flush the inlet line and flow cell with the test solution for approximately 1 minute. This primes the inlet line and the flow cell with the test solution for the forthcoming analysis and should be performed prior to the start of the run and in-between runs immediately prior to any analysis. After 1 minute click “pause pump” and exit the “setup and focus” screen.

The context file establishes the appropriate operating run conditions within the Visual Spreadsheet. To open an existing Context File or Create a new Context File for sample analysis perform the following:

To load an existing Context File in the Visual Spreadsheet click the “context” tab in the “setup” pull down menu and click the “Load” tab. In this window click “Load a Context File”.

Select an appropriate Context File with the mouse from those available in the Context File menu (sorted by sample size) and click “OK”. Confirm by clicking “OK”. Review pre-set parameters within the Context File under the individual tabs such as the “sample volume”* under the “fluidics” tab and the min-max particle diameter under the “Filter” tab to make sure the settings are appropriate for your sample analysis.

Once all settings are established, if changes are required, make the necessary changes (refer to the FlowCam® system manual for optimal operating conditions) and exit the context window by pressing “OK”, you will be prompted to enter the “reason for changes” then press “Accept”.

To create a new Context File (This should only be performed by a system administrator). Refer to the FlowCam® system manual for optimal operating conditions) click on “Visual Spreadsheet” then “Setup” then “Context”. Populate the appropriate run settings under each tab within the context window.

After all changes are made Click “Save Context File As” under the “Load” tab, enter the “File Name” and click OK. Include the file name, the sample size, date created and initials of the person creating the file. Proceed to the following Sample Analysis Step.

### Adjustments to the Optical Path and the Focal Length of the Camera in Example 2.

The accuracy of the instrument is based on proper alignment of the camera with the flow cell (optical path) and proper focal length (depth of focus of the objective lens within the flow cell). The instrument will maintain proper alignment between sample runs and between calibrations. Calibration and System suitability will verify proper alignment of the instrument.

If the instrument fails Calibration notify laboratory supervision. If adjustments are necessary refer to the FlowCam® system manual for optimization of the Optical path and the Focal Length of the objective lens.

### Sample Analysis in Example 2.

Left click twice on the “visual spreadsheet icon to open. Select “analyze”, under “AutoImage Mode”. If changes were made to the “Context” file the instrument will ask “Do you want to use the modified context settings for the analysis?” click “Yes”. Under “Notes” enter sample information, study reference, analyst & date.

Enter file name (Data entry in this field should include the test reference under the file heading ex. “4× List” File names must include sample information and the test date; click “OK”, confirm by clicking “OK” a second time.

The analysis will proceed according to the run conditions described in the context file.

The instrument is very sensitive to vibrations during analysis. Bench top vibrations trick the instrument into falsely recognizing stationary flow cell contamination as particles. These false signals lead to analysis disruptions and bad data requiring the analyst to repeat the run. Bench top vibrations must be minimized while the analysis is taking place. If the instrument is capturing false signals (recognized by repetitive images of long bands in the view window or repetitive images of the same particle) the instrument requires immediate “recalibration”. To recalibrate click on the tools pull down menu and click recalibrate. This will reset the background and re-train the instrument. Runs with these false signals may still be processed with the correct filter.

### Post Run Analysis in Example 2:

### Data Sorting.

The FlowCam® system is currently utilized as a laboratory tool to collect data on particle counts and to record images of these particles to aid in describing particle morphology.

Load the individual sample run for processing by opening the data file from the file menu. The data file is displayed in the visual spreadsheet window.

### Data Review

Within the visual spreadsheet data is presented and sorted in each of four windows. The presentation of data within these four windows may be changed to present particle identified in the sample analysis by size, shape and frequency (refer to the Operations manual for instructions on data sorting).

By right clicking in each window a drop down menu will allow changes to the data presentation. For example by right clicking a window then clicking the “histogram” menu and selecting the tab “circle fit frequency” a bar chart will appear in the window with the distribution of particles based on their comparison to a perfect sphere (1.0 being a perfect sphere). This comparison is used to isolate and identify particles such as air bubbles and silicone oil which present as nearly perfect spheres in aqueous solutions.

By right clicking on the same window and selecting the “scatter plots” menu and clicking “diameter vs. circle fit” the same data is displayed as a scatter plot where each red dot represents a particle counted by the instrument. These are sorted by diameter on the X-axis and the circle fit (1.0 being a perfect sphere) on the Y-axis.

By left clicking and dragging the mouse highlighting a select group of red dots on the scatter plot two new windows will open, the first is a summary window with details related to total number and statistical analysis of the highlighted particles. The second is the images of the selected particles themselves.

This technique can be used to identify for example all particles of a certain size and shape in a particular sample (For example silicone oil droplets are typically between 2-30 μm and have a circle fit >0.8).

MFI is used to measure the number of the particles of the container and the container containing the drug at t=0. Then measurement of the number of the particles is taken at different time-intervals to evaluate the increased particle count. In addition, MFI enables an evaluation of particle morphology. Using MFI, one can determine if the particles are associated with the container or with the drug. In the case of a biological drug, MFI can identify aggregated proteins which has a specific morphology which container particles don't have.

### Example 3—Prophetic Example

SiO2 Medical Products, Inc. Containers Lower the Immune Responses to OVA

This example shows that the containers (in this example, syringes) of this invention are expected to lower unwanted immune responses to certain drugs or proteins (e.g. ovalbumin (OVA)) as compared to borosilicate glass containers (in this example, syringes) with silicone oil as the lubricant.

The syringes of the invention are staked-needle, 1 mL long syringe barrels made of cyclic olefin polymer (COP) with a trilayer or quadlayer barrier coating system on the interior surface defining a lumen. The trilayer barrier coating system is deposited as a series of three independent coatings—an adhesion or tie coating or layer, a barrier coating or layer, and a pH protective coating or layer—under the conditions and using the materials specified below:

| Gas | MW | units | | Units | Adhesion | Barrier | Protective |
|---|---|---|---|---|---|---|---|
| HMDSO | 162.38 | g/mole | Power | W | 100 | 150 | 100 |
| TMDSO | 134.32 | g/mole | O2 Flow | sccm | 4 | 200 | 4 |
| O2 | 16 | g/mole | HMDSO Flow | sccm | 0 | 2 | 0 |
| Ar | 40 | g/mole | TMDSO Flow | sccm | 8 | 0 | 8 |
| | | | Argon Flow | sccm | 95 | 0 | 95 |
| | | | W/FM | KJ/kg | 27133 | 57025 | 27133 |
| | | | W/FM | J/kg | 2.71E+07 | 5.70E+07 | 2.71E+07 |

Where a quadlayer barrier coating system is used, the trilayer coating specified above is applied, followed by a fourth, lubricity layer formed by introducing octamethylcyclotetrasiloxane—OMCTS—as a precursor, reacted with a concurrent flow of oxygen to form a crosslinked, lubricious coating firmly attached to the trilayer barrier coating system.

The plunger used is a commercially available Datwyler Omniflex plunger composed of a bromobutyl rubber core and a fluoropolymer conformal coating is applied to the exterior surface of the plunger to block potential leachates into the drug.

The borosilicate glass syringes used as a comparative example are BD Hypak™ Glass Prefillable Syringes with Fixed Needle. The syringe is a 1 ml long staked needle syringe with a 27 gauge thin wall needle and used with a Datwyler Omniflex Plunger, siliconized according to normal commercial pharmaceutical practice with silicone oil.

The OVA samples for injection are prepared at 0.25 mg/mL in 20 mM sodium phosphate buffer (pH 7.4) containing 9% (w/v) sucrose.

Materials used to prepare samples for injection are of United States Pharmacopeia grade or higher. OVA is purchased from Fisher Scientific (Waltham, Massachusetts) and/or OVA Laboratories, Inc. (Wilmington, Massachusetts).

Clinical protocol: Adult female CB6F1 (BALB/c×C57 BL/6) mice greater than six weeks of age are employed, which can be purchased from Charles River. Laboratories, Inc. (Wilmington, Massachusetts). Four or five mice are housed in each sterile, air-filtered cage with food and water available ad libitum. Mice are allowed to acclimate for a minimum of one week before the start of the study. Subcutaneous injections are administered to mice in the scruff of the neck on days 1 and 15. Samples are administered using non-siliconized syringes, and each injection of 200 μL contained 50 μg of OVA. Groups of five to eight mice are treated with OVA, OVA that contains emulsified silicone oil microdroplets, OVA that contains syringe-extracted silicone oil microdroplets, and OVA that contains alum microparticles. Also, control groups of mice are injected with buffer or with protein-free buffer that contains emulsified silicone oil microdroplets. The first group of mice are treated with OVA in glass syringes with silicone oil as the lubricant and the second group of mice are treated with OVA in the syringes of this invention which is free of silicone oil.

### Antibody Testing Protocol in Example 3:

Submandibular blood draws are performed before the start of the study to serve as a baseline for each mouse, as well as on day 11 and 29 to capture primary and secondary immune responses. Blood samples are collected in sterile microcentrifuge tubes and placed on ice. Subsequently, samples are centrifuged at 15,000 rpm for 10 min at 4° C. Serum is then obtained and stored in aliquots at −80° C. until further analysis.

Antibodies specific to OVA are measured using indirect ELISA. Immulon® 4HBX plates are coated with 10 μg/mL OVA in 20 mM Tris at pH 8.5 (100 μL/well) and incubated overnight at room temperature with gentle agitation. Plate wells are drained and then treated with 300 μl of blocking solution (PBS (pH 7.4), 2% BSA, 0.05% Tween 20*) for 1.5 h. Plates are washed three times with wash buffer (PBS, 0.05% Tween 20®) using an EL×50 plate washer (BioTek, Winooski, Vermont). Dilution buffer (PBS (pH 7.4), 2% BSA, 0.05% Tween 20®) is then added to plates (50 μL/well). Serum samples are pretreated in 300 mM acetic acid for 1 h.⁴¹ After pH adjustment to 7.4 with 1 M Tris buffer (pH 9.5), 50 μL of diluted serum samples are immediately transferred to the first row of the plate. Samples are serially diluted down the plate in dilution buffer and incubated for 1 h. Plates are washed five times with wash buffer. Next, goat anti-mouse antibodies conjugated to horseradish peroxidase of subclass IgG1, IgG2a, IgG2b, IgG2c, IgG3, or IgM are diluted in blocking solution and added to the wells (50 μL/well). The CB6F1 (the F1 generation from a BALB/c×C57BL/6 cross) mouse strain can produce both IgG2a and IgG2c immunoglobulin isotypes as the IgG2a immunoglobulin isotype is encoded by the parental BALB/c mouse strain and the IgG2c isotype is derived from the C57BL/6 strain. After secondary incubation for 1 h, the plates are washed five times. Substrate solution 1-Step™ Ultra TMB is added (50 μL/well). After 25 min, the reaction is stopped by addition of 30 μl of 0.5 M sulfuric acid. Absorbance is measured at 450 nm using a Vmax® microplate reader (Molecular Devices Corporation, Sunnyvale, California). Absorbance values are used to determine endpoint titers for each mouse. We define endpoint titer in this Example as the reciprocal of the highest dilution that gives a signal above the cutoff. Cutoff values are calculated for each mouse using pretreatment blood drawn at day 0 and a statistically defined endpoint titer determination method.

The unwanted immune response is characterized by the number of mice in each group after the final injection at day 29 showing anti-OVA antibodies.

The results show that more mice injected with OVA in the glass syringes with silicone oil develop one or more of anti-OVA IgG1, IgG2a and IgG3 antibodies, compared to the mice injected with OVA in the syringes of this invention free of silicone oil.

### Example 4—Working Example

SiO2 Medical Products, Inc. Containers Lower the Particle Count
Resulting from Particle Activation, Versus Borosilicate Glass Containers

Example 4 is a summary of experimental work carried out by Carly F. Chisholm, William Behnkel, Yekaterina Matskiv, and Theodore W. Randolph, each from the Center for Pharmaceutical Biotechnology, Dept. Chemical and Biological Engineering, University of Colorado, Boulder, Colorado 80309, and Ashley A. Frazer-Abel from Exsera Biolabs, Anschutz Medical Campus, Aurora, Colorado, 80045. They have written a paper entitled, “Subvisible Particles in IVIg Formulations Activate Complement in Human Serum.” Funding for this work was provided by the NIH under RO1 EB006006, and by SiO2 Medical Products, Inc., which provided the test protocol carried out in this experimental work.

Materials used for Example 4 were of USP grade or higher. Intravenous immunoglobulin (IVIG; GAMMAGARD LIQUID®, Shire US Inc., Lexington, MA) was purchased from Wardenburg Pharmacy at the University of Colorado at Boulder. Chemicals purchased from Sigma Aldrich (St. Louis, Missouri) included sodium phosphate monobasic, sodium phosphate dibasic, and glycine. Chemicals purchased from Fisher Scientific (Waltham, Massachusetts) included polysorbate 20 (Tween 20™, N.F., Multi-Compendial, J.T. Baker), 10× phosphate buffered saline, and HyClone™ water for injection (WFI). Siliconized glass syringes were BD Hypak SCF 1 mL long 27G1/2 (BD Medical-Pharmaceutical Systems, Franklin Lakes, NJ). SiOPlas™ syringes (1 ml), (SiO2 Medical Products, Inc., Auburn, AL) were composed of cyclic olefin polymer (COP) syringe barrels whose interior surfaces were coated with a silica-based barrier coating system and a crosslinked organosiloxane lubricant as described in Example 3, both applied by plasma enhanced chemical vapor deposition. SiOPlas™ vials (6 ml) were composed of COP that was coated on the vial interior with a barrier coating system. 6 mL Ompi EZ-fill® borosilicate glass vials (Schott, AG) were provided by SiO2 Medical Products, Inc. (Auburn, AL).

### Protein Formulation

Prior to use, IVIg was formulated at a protein concentration of 1 mg/mL in either phosphate-buffered saline at pH 7.4 (PBS), or in 250 mM glycine pH 4.25 with 0.02% (v/v) polysorbate 20 (PS20). To remove any pre-existing particulates, GAMMAGARD LIQUID® samples containing 100 mg/mL immunoglobulin G in 250 mM glycine were centrifuged at 20,000×g for 20 minutes at 4° C. and the supernatant was used as a stock solution. The IVIg stock solution was diluted 1:100 into either 0.22 micron filtered PBS pH 7.4 or 250 mM glycine pH 4.25 with 0.02% (v/v) polysorbate 20 to yield a final concentration of 1 mg/mL IVIg.

### Accelerated Stress Testing of IVIg Formulations Using Agitation

Siliconized glass and SiOPlas™ syringes, the latter 1 mL long syringes made as described in Example 3, were filled with 1 mg/mL IVIg in 250 mM glycine pH 4.25 with 0.02% (v/v) PS20. Syringes were filled so that a uniform headspace gap of 4 mm was present between the liquid and the stopper (Datwyler Pharma Packaging, Pennsauken, NJ). These syringes were rotated end-over end at room temperature for 10 days, causing the air bubble in the headspace gap to move from one end of syringe to other end with each rotation. In a few of the syringes that were tested, the air bubble became lodged at one end of the syringe and failed to move as the syringe was rotated. These syringes were excluded from further analysis. After the 10 days of end-over-end rotation, each formulation was expelled from the syringe through the needle using an automated syringe pump at 150 mm/min and sample was collected in pre-rinsed polypropylene tubes and tested for sub-visible particle concentrations and for complement activation capacity.

Another set of identical siliconized glass and SiOPlas™ syringes were filled with 1 mg/mL IVIg in PBS. These syringes were placed horizontally on an orbital shaker and shaken overnight at room temperature. Following shaking, the formulations were expelled from the syringes through the needle using an automated syringe pump at 150 mm/min, collected in pre-rinsed polypropylene tubes, and tested for sub-visible particle concentrations and complement activation capacity.

### Accelerated Stress Testing of IVIg Formulations Using Freeze Thawing

Borosilicate vials (6 mL) and SiOPlas™ vials (6 mL) made as described in Example 1 were filled with 4 mL of a formulation containing 1 mg/mL IVIg in PBS pH 7.4. The contents of the vials were subjected to 1 or 6 freeze-thaw cycles. In each freeze-thaw cycle, the vials were first dipped in liquid nitrogen for 2 minutes, then thawed in a water bath at 30° C. for 14.5 minutes. Each vial was swirled gently for mixing prior to next freeze-thaw cycle.

### Analysis of Sub-Visible Particle Concentrations

Particle concentrations and images of particles in the various stressed formulations were obtained using flow imaging microscopy (FlowCAM®, Fluid Imaging Technologies, Scarborough, Maine), as previously described. 15 Samples that had been subjected to 6 freeze-thaw cycles contained concentrations of particles that approached or exceeded the upper limit for the flow imaging microscopy instrument, and so these formulations were diluted 100-fold with phosphate buffered saline, pH 7.4 before analysis. Particle counts were measured for formulations expelled from individual syringes and vials that were tested for complement activation.

### Analysis of Soluble Protein Fractions by Size Exclusion Chromatography

Size exclusion chromatography was used to monitor the retention of monomeric protein and the appearance of any soluble aggregates in IVIG samples after application agitation or freeze-thawing stresses. A TSKgel G3000SWXL column (TOSOH Biosciences, Montgomeryville Pennsylvania) was used with an Agilent 1100 series system (Santa Clara, CA). The eluent was monitored at an absorbance of 280 nm in the Agilent ChemStation software. The mobile phase of 100 mM sodium sulfate, 100 mM sodium phosphate, and 0.05% (w/v) sodium azide at pH 6.7 was passed through the system at 0.6 mL/min. Peak areas in chromatograms were quantified in GRAMS/AI software version 9.1 (Thermo Fisher Scientific Inc., Waltham, Massachusetts).

### Complement Activation in Human Serum Samples

Samples pooled from three vials or syringes of the various stressed IVIg formulations as well as unstressed control samples of each formulation were delivered to Exsera Biolabs (Denver, CO) for analysis of their capacity to activate complement. Complement activation was measured in normal human serum pooled from three individual donors that had been previously screened for normal complement function. Test samples of stressed IVIg formulations were diluted tenfold into pooled human serum, mixed and allowed to incubate at 37° C. for 30 minutes. After incubation, samples were stored at −80° C. until further testing was conducted. For analysis of complement activation, concentrations of four complement cascade proteins, C3a, Bb, C4a and C5a, were measured by ELISA using kits purchased from Quidel Corporation (San Diego, CA). C4a was chosen because it is a marker of activation of the classical or lectin pathway, Bb as a distinctive marker of the alternative pathway for complement activation, C3a as the central point of complement activation and C5a as a marker of terminal complement pathway activation.40 Triplicate ELISA measurements were conducted for each of the for complement cascade proteins. In addition to testing of stressed IVIG samples, several controls were analyzed. These control samples contained pooled serum alone, or samples of serum to which saline solution, phosphate buffered saline with zymosan or phosphate buffered saline with heat aggregated gammaglobulins were added in a 1:9 ratio. The average of the measurements were plotted versus particle sample counts as a fold increase compared to concentrations measured in the saline control.

As expected, each of the accelerated stress testing methods generated microparticles within the tested formulations (Table 1). Freeze thawing of IVIg in PBS pH 7.4 resulted in the highest number of subvisible particles. A single freeze-thaw cycle produced 3.2×10⁵ and 7.1×10⁵ particles of size greater than 2 micron in borosilicate and SiOPlas™ vials, respectively. Particles larger than 10 microns were also produced, with 8×10³ particles detected in borosilicate vials and 5.1×10⁴ particles found in SiOPlas™ vials.

Application of multiple freeze thaw cycles further increased particle numbers by about an order of magnitude. After six cycles, particle concentrations in borosilicate vials and SiOPlaS™ vials had increased to 4.8×10⁶ and 2.1×10⁶ particles/mL of size greater than 2 microns, respectively. Particles larger than 10 micron were also generated, with 2.3×10⁵ and 3.6×10⁵ in borosilicate and SiOPlas™ vials, respectively.

The number of particles produced as a result of agitation stresses depended on the container type and also on the type of agitation that was applied⁴¹. Overnight agitation of IVIg formulations in PBS pH 7.4 using an orbital shaker generated 2.4×10⁶ particles of size greater than 2 microns in siliconized glass syringes, and 6.0×10⁵ particles/mL in corresponding SiOPlas™ syringes. As was the case with the freeze-thaw studies, roughly an order of magnitude fewer large particles (>10 micron) were formed in syringes of both types.

End-over end rotation for ten days was the gentlest accelerated stability test that was applied. After IVIg formulations in glycine pH 4.25 underwent end-over-end rotation for 10 days, only 3×10³ and 1.03×10⁵ particles/mL in the particle size range greater than 2 microns were detected in SiOPlas™ and siliconized glass syringes, respectively. Correspondingly few particles larger than 10 micron were also generated, with 2.0×10³ and 90 particles/mL detected in siliconized glass and SiOPlas™ syringes, respectively.

Collections of typical flow imaging microscopy images of particles produced by the various accelerated stress methods are presented in FIG. 1. Samples stressed by freeze-thawing in vials predominately contained aspherical microparticles characteristic of proteinaceous aggregates (FIG. 1a), whereas samples stressed by agitation in siliconized glass syringes (FIG. 1b) contained numerous spherical particles characteristic of droplets of lubricants. Agitation in the SiOPlas™ syringes (FIG. 1c) produced particles of various morphologies that may have comprised both irregularly-shaped protein aggregates and spherical droplets of silicone oil.

For all of the accelerated stress conditions tested, size exclusion chromatographic analysis showed that only minimal (<5%) amounts of insoluble protein were formed. No soluble aggregates were detected under any condition. Application of six freeze-thaw cycles to IVIg in borosilicate glass vials produced the highest numbers of particles per mL—nearly 5 million particles greater than 2 microns—but this represented a loss of only 3.7% of the original monomeric protein.

### Complement Activation in Human Serum in Response to Particles Produced Under Accelerated Stress Conditions.

Particles of size greater than 2 microns within IVIg formulations that had been subjected to accelerated stress conditions activated complement in a linear, dose-dependent fashion when the IVIg formulations were diluted ten-fold into human serum. Particles of size greater than 10 microns within IVIg formulations did not correlate with activated complement.

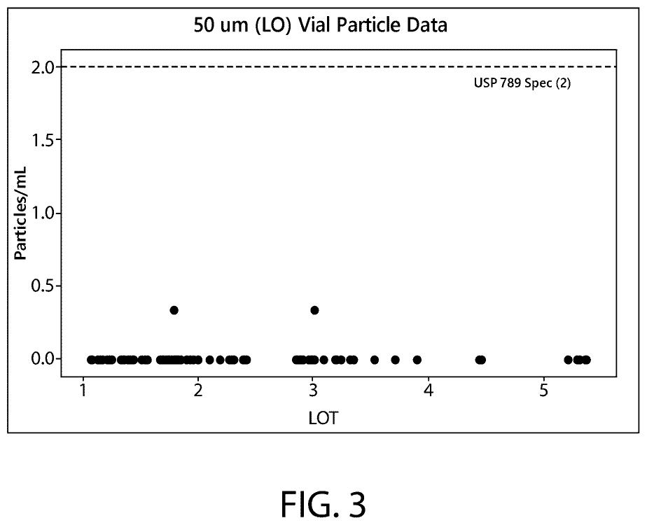

The observed complement activation was consistent with activation through the alternative pathway. No increase over saline control levels was seen for C4a, which is a marker for the classical or lectin pathways (FIG. 2). In contrast, Bb, a marker of the alternative pathway for complement activation, increased linearly (r²=0.94) as the concentrations of particles in the size range greater than 2 microns increased (FIG. 3).

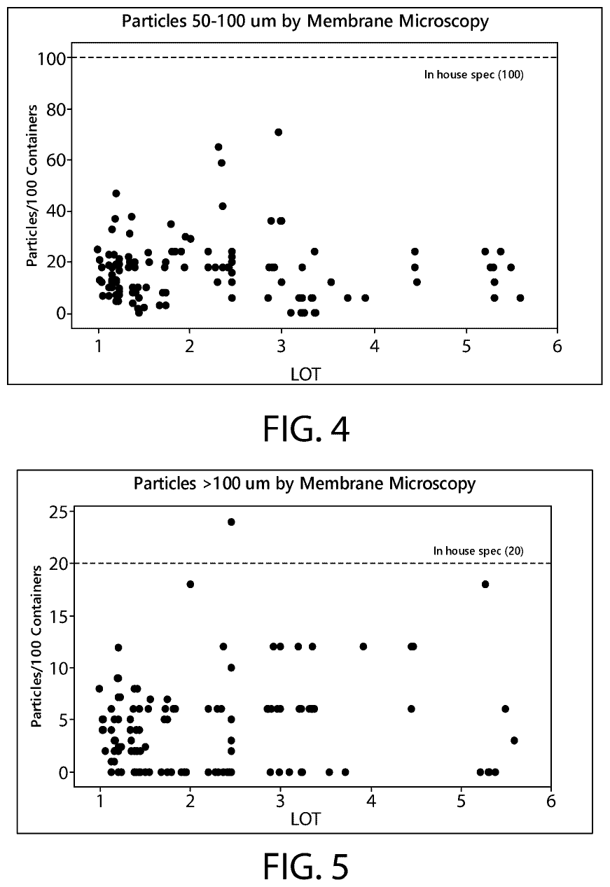

Increases in concentrations of the anaphylatoxins C3a and C5a were also observed when particles were diluted into serum (FIGS. 4 and 5). As with the Bb response, the fold increases vs. saline control were linearly dependent on particle dose, with correlation coefficients r² of 0.85 and 0.99 for C3a and C5a, respectively. Responses to the IVIg formulations that were subjected to 6 freeze-thaw cycles in borosilicate glass vials produced increases in C3a and C5a concentrations that were 2.4 and 8.9 fold higher than those observed in saline controls.

For comparison, positive controls containing 1 mg/mL zymosan or 1 mg/L heat aggregated gamma globulins stimulated ca. 11-fold increases (vs. saline) of C3a, Bb, and C4a, and ca 32-fold increases (vs. saline) in C5a levels.

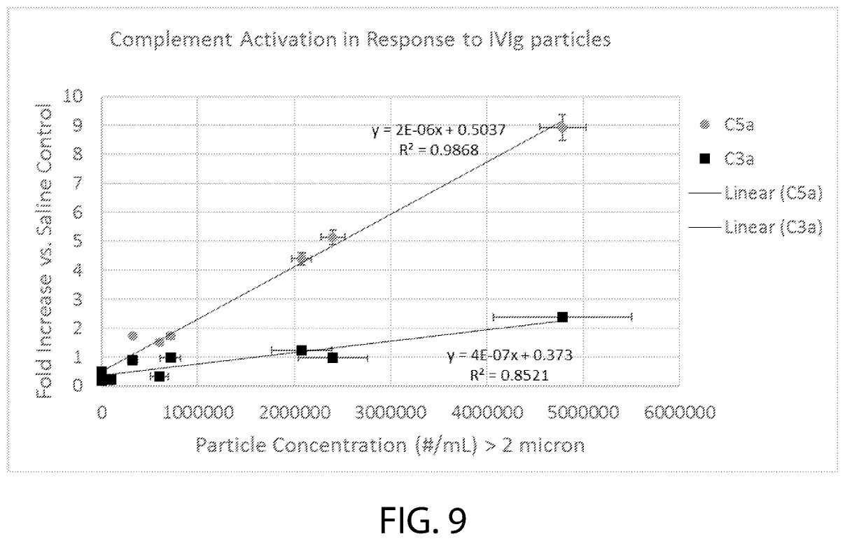

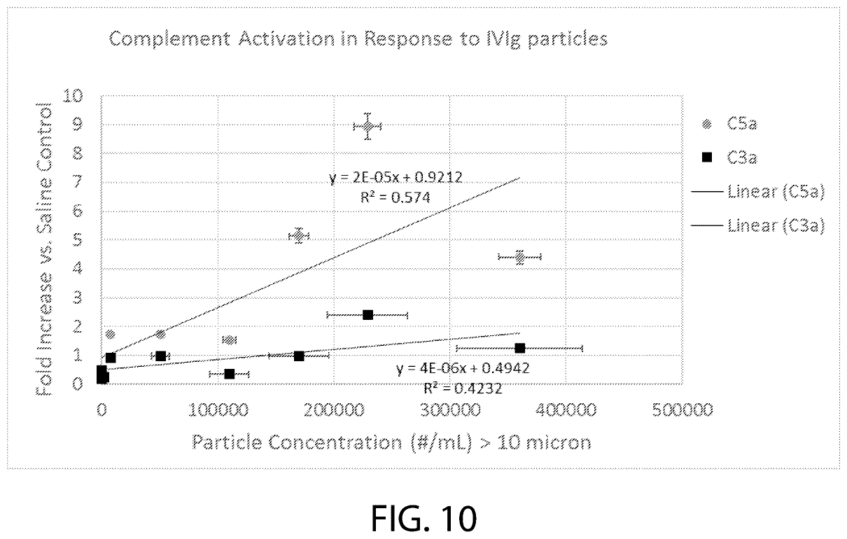

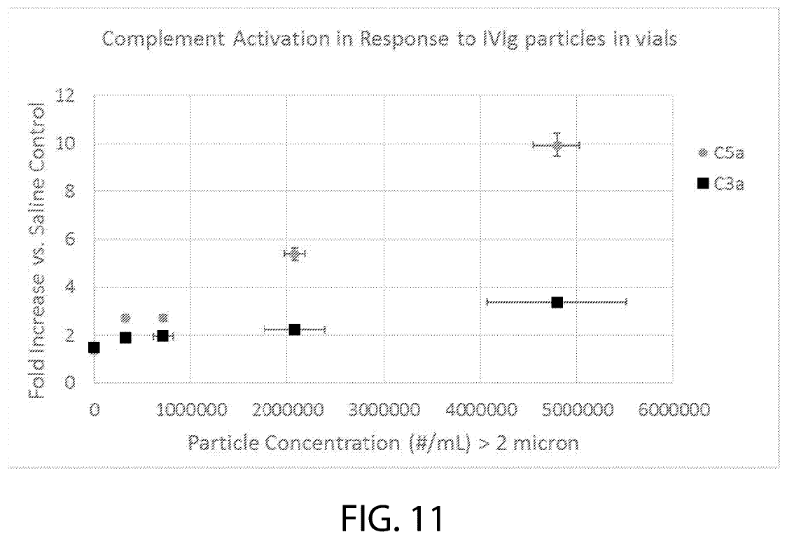

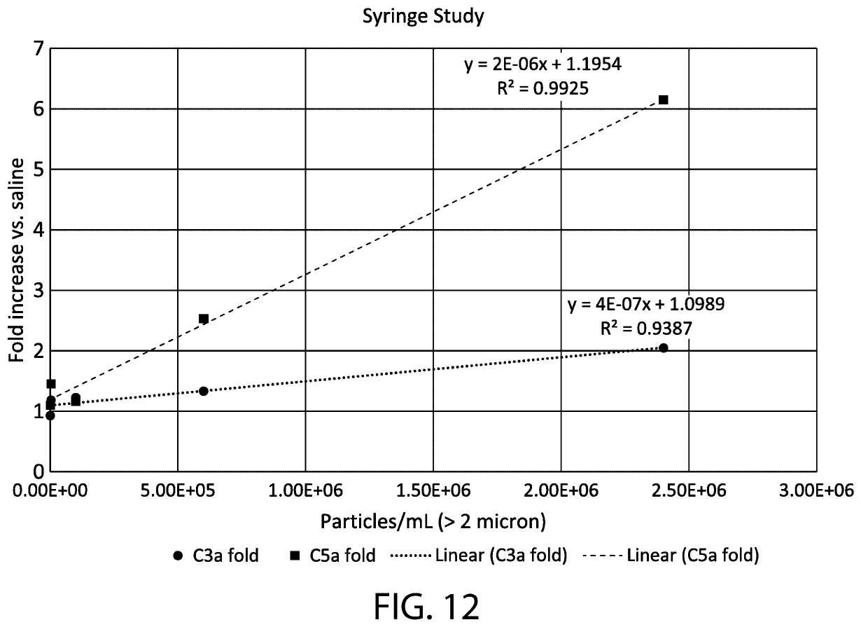

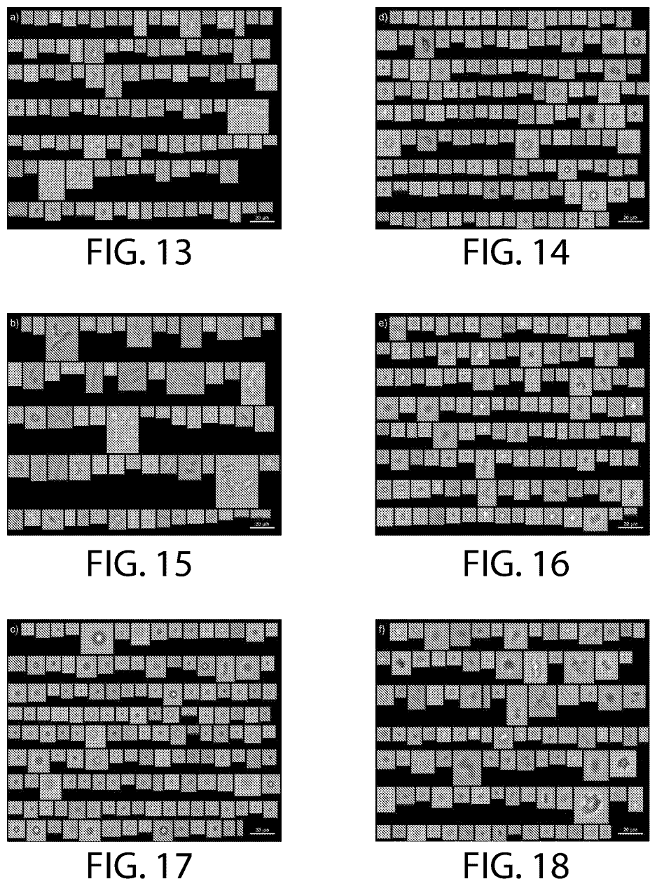

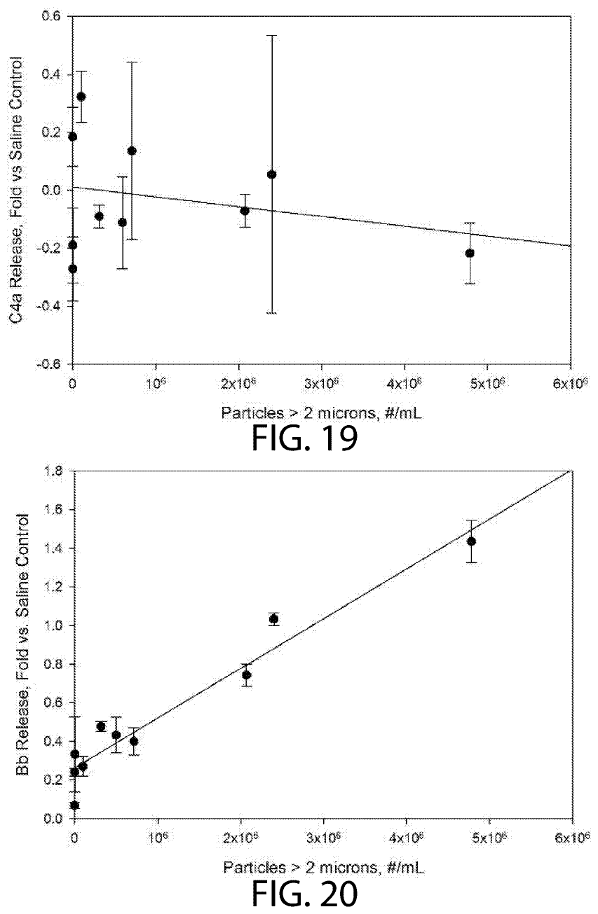

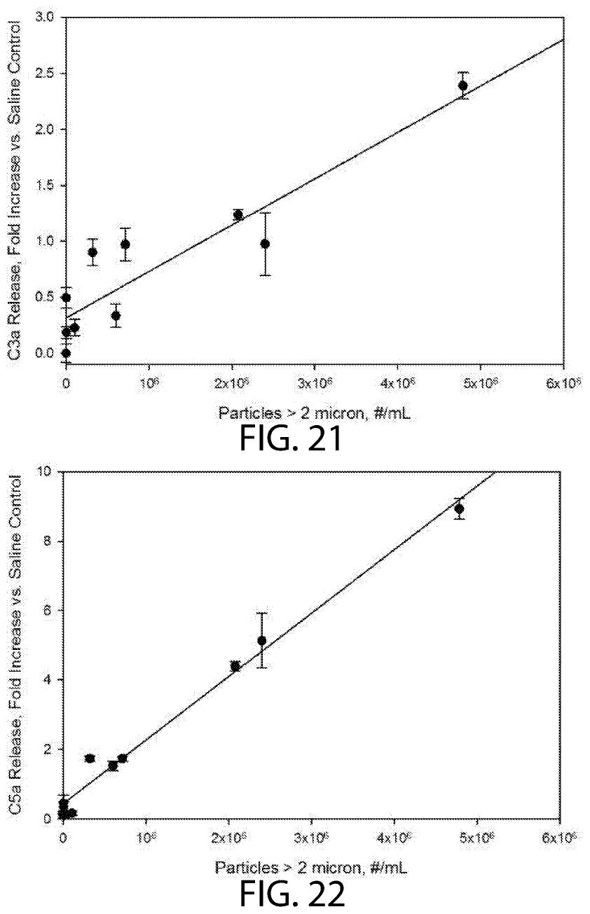

The experimental results are tabulated in Tables 1-2 and FIGS. 9-22. Note that the first row of data in Table 2 does not correspond to any data in Table 1, but otherwise the respective tables are different presentations of the same experimental data. Table 1 shows concentrations of subvisible particles in stressed samples submitted for complement activation testing. Samples were pooled from three samples from each stress/container combination. Container-to-container variation in particle concentrations in samples prior to pooling was <15%.

TABLE 1

| | Container Type/ | Particles of size >2 | Particles of size >10 |
|---|---|---|---|
| Applied Stress | Buffer Condition | micron, #/mL | micron, #/mL |
| Unstressed Control | Polypropylene/250 | 300 | 10 |
| | mM glycine pH 4.25, | | |
| | 0.02% (v/v) | | |
| | polysorbate 20 | | |
| Single Freeze Thaw | SiOPlas ™ Vial/250 | 7.1 × 10⁵ | 5.1 × 10⁴ |
| Cycle | mM glycine pH 4.25, | | |
| | 0.02% (v/v) | | |
| | polysorbate 20 | | |
| Single Freeze Thaw | Borosilicate Glass | 3.2 × 10⁵ | 8.0 × 10⁴ |
| Cycle | Vial/250 mM | | |
| | glycine pH 4.25, | | |
| | 0.02% (v/v) | | |
| | polysorbate 20 | | |
| Six Freeze Thaw | SiOPlas ™ Vial/250 | 2.1 × 10⁶ | 3.6 × 10⁵ |
| Cycles | mM glycine pH 4.25, | | |
| | 0.02% (v/v) | | |
| | polysorbate 20 | | |
| Six Freeze Thaw | Borosilicate Glass | 4.8 × 10⁶ | 2.3 × 10⁵ |
| Cycles | Vial/250 mM | | |
| | glycine pH 4.25, | | |
| | 0.02% (v/v) | | |
| | polysorbate 20 | | |
| Orbital Shaker, | SiOPlas ™ Syringe/ | 2.4 × 10⁶ | 1.7 × 10⁵ |
| Overnight | PBS pH 7.4 | | |
| Orbital Shaker, | Siliconized Glass | 6.0 × 10⁵ | 1.1 × 10⁵ |
| Overnight | Syringe/PBS pH 7.4 | | |
| End-over-end | SiOPlas ™ Syringe/ | 3 × 10³ | 90 |
| rotation, 10 days | 250 mM glycine pH | | |
| | 4.25, 0.02% (v/v) | | |
| | polysorbate 20 | | |
| End-over-end | Siliconized Glass | 1.0 × 10⁵ | 2 × 10³ |
| rotation, 10 days | Syringe/250 mM | | |
| | glycine pH 4.25, | | |
| | 0.02% (v/v) | | |
| | polysorbate 20 | | |

TABLE 2

| | | | Particle | Particle | |
|---|---|---|---|---|---|
| | | | Concentration | Concentration | Meets USP |
| Sample Name | Sample Details | Container Type | (#/mL) >2 micron | (#/mL) >10 micron | <788>/<787> |
| Control Article | 1 mg/mL IVlg in PBS pH 7.4 | none | 3000 | 300 | Meets |
| Test Article #1 | 1 FT of 1 mg/mL IVIg in PBS | Silica-coated COP | 714000 | 51000 | Meets |
| | pH 7.4 | vial | | | |
| Test Article #2 | 1 FT of 1 mg/mL IVIg in PBS | Borosilicate glass | 320000 | 8000 | Meets |
| | pH 7.4 | vial | | | |
| Test Article #3 | 6 FT of 1 mg/mL IVIg in PBS | Silica-coated COP | 2075000 | 360000 | Exceeds |
| | pH 7.4 | vial | | | |
| Test Article #4 | 6 FT of 1 mg/mL IVIg in PBS | Borosilicate glass | 4785600 | 229000 | Exceeds |
| | pH 7.4 | vial | | | |
| Control Article | 1 mg/mL IVIg in PBS pH 7.4 | none | 300 | 10 | Meets |
| Test Article #1 | 1 mg/mL IVIg in siliconized | Siliconized glass | 2400000 | 170000 | Exceeds |
| | glass syringes shaken | syringe | | | |
| | overnight | | | | |
| Test Article #2 | 1 mg/mL IVIg in quadlayer | Quadlayer SiO2 | 600000 | 110000 | Meets |
| | SiO2 syringes shaken | syringe | | | |
| | overnight | | | | |
| Test Article #3 | 1 mg/mL IVIg in quadlayer | Quadlayer SiO2 | 3000 | 90 | Meets |
| | SiO2 syringes rotated end- | syringe | | | |
| | over-end | | | | |
| Test Article #4 | 1 mg/mL IVIg in siliconized | Siliconized glass | 103000 | 2000 | Meets |
| | glass syringes rotated end- | syringe | | | |
| | over-end | | | | |

Looking more particularly at the vial results, after 1 single freeze thaw, complement activation in IVIg samples in borosilicate and in SiO2 vials were not significantly different from that provoked by initial IVIg solution, and differences between borosilicate and SiO2 vials were not statistically significant. Large, statistically significant increases in complement activation were seen after six freeze thaws. Complement activation, as determined by measured levels of both C3a and C5a, was significantly higher in terms of IVIg freeze-thawed in borosilicate vials compared to SiO2 vials. Responses to IVIg samples in borosilicate vials treated with six freeze thaws (3.3+/−0.1 and 9.9+/−0.3 times greater than C3a and C5a responses to saline controls) were in a range that is typically associated with adverse infusion reactions such as facial flushing a potential anaphylaxis. Initial particle levels in IVIg preparation were 3000/mL greater than 2 micron, and 300/mL greater than 10 micron. After a single freeze thaw, particle levels in SiO2 vials were 7.1×10⁵ per mL greater than 2 microns, and 5.1×10⁴ greater than 10 microns; whereas in borosilicate vials the counts were lower, 3.2×10⁵ and 8×10³ for particles of size greater than 2 microns and greater than 10 microns, respectively. After six freeze thaws, particle levels in SiO2 vials were 2.1×10⁶ per mL greater than 2 microns, and 3.6×10⁵ greater than 10 microns; whereas in borosilicate vials the counts were now higher, 4.8×10⁶ and 2.3×10⁵ for particles of size great than 2 microns and greater than 10 microns, respectively.

Looking more particularly at the syringe results, complement activation was highly linear in particle content (r²>0.99) for both C3a and C5a. At each condition, complement activation, as determined by measured levels of both C3a and C5a, was significantly higher in siliconized glass syringes compared to SiO2 quadlayer syringes. Responses to IVIg samples in borosilicate vials treated with six freeze thaws (3.3+/−0.1 and 9.9+/−0.3 times greater than C3a and C5a responses to saline controls) were in a range that is typically associated with adverse infusion reactions such as facial flushing a potential anaphylaxis. After overnight agitation on shaker table, particle levels in SiO2 syringes were 6×10⁵ per mL greater than 2 microns; whereas in siliconized glass syringes the counts were 2.4×10⁶ per mL. After 10 days end-over-end rotation, particle levels in SiO2 syringes were 3×10³ per mL great than 2 microns; whereas in siliconized glass syringes the counts were 1.0×10⁵.

The following conclusions were reached from the experimentation of Example 4. First, complement activation (C5a and C3a) was directly proportional to particle concentrations for the 2-10 micron particle size range. Second, complement activation was independent of how the particles were generated including formulation pH, stress applied, and presence of surfactant. Third, for identical formulations and stresses applied, SiO2 Medical vials and syringes had generally lower particle levels than borosilicate vials and siliconized glass syringes.

1.-61. (canceled)

62. Use of a container comprising:
a thermoplastic wall having an internal surface defining a lumen; and
a PECVD drug-contact coating on or adjacent to the internal surface and positioned to contact a fluid in the lumen, in which the drug-contact coating consists essentially of SiOₓC_{y}H_{z}, in which
x is between 0.5 and 2.4 as measured by x-ray photoelectron spectroscopy (XPS),
y is between 0.6 and 3 as measured by XPS, and
z is between 2 and 9 as measured by Rutherford backscattering; and
as a primary drug container in which
a polypeptide composition is contained in the lumen in contact with the PECVD drug-contact coating; and
the primary drug container contains less than 10000 particles having effective spherical diameters between 2 and 50 micrometers (μm) per mL of solution, measured by light obscuration particle count testing or dynamic image analysis.

63. The use of the container of claim 62, wherein the primary drug container is a syringe, cartridge, or vial.

64. The use of the container of claim 63, in which the thermoplastic is selected from COC, COP, polypropylene, PET, polycarbonate, and polystyrene.

65. The use of the container of claim 64, in which the drug contact coating comprises:
a tie coating or layer comprising or consisting of SiOxCyHz in which x is from about 0.5 to about 2.4 as measured by X-ray photoelectron spectroscopy (XPS), y is from about 0.6 to about 3 as measured by XPS, and z is from about 2 to about 9 as measured by at least one of Rutherford backscattering spectrometry (RBS) or hydrogen forward scattering (HFS), the tie coating or layer having an outer surface facing the wall surface and the tie coating or layer having an interior surface;
a barrier coating or layer of SiOx, in which x is from about 1.5 to about 2.9 as measured by XPS, the barrier coating or layer positioned between the interior surface of the tie coating or layer and the lumen; and
a pH protective coating or layer of SiOxCyHz, in which x is from about 0.5 to about 2.4 as measured by XPS, y is from about 0.6 to about 3 as measured by XPS, and z is from about 2 to about 9 as measured by at least one of RBS or HFS, positioned between the barrier coating or layer and the lumen.

66. The use of the container of claim 65, having lower particle levels than borosilicate and siliconized glass containers of the same size, optionally under stress conditions.

67. The use of the container of claim 65, in which the drug contacting coating further comprises a lubricity coating or layer of SiOxCyHz on top of the pH protective coating, in which x is 0.5-2.4, y is 0.6-3, x and y being measured by x-ray photoelectron spectroscopy (XPS), and z is 2-9, z being measured by Rutherford backscattering analysis.

68. The use of the container of claim 67, in which the lubricity coating is prepared by PECVD using octamethylcyclotetrasiloxane (OMCTS) as the organosilicon precursor.

69. The use of the container of claim 62, in which the internal surface is generally cylindrical, the primary drug container further comprising a plunger positioned and slidable within the lumen.

70. The use of the container of claim 69, in which the plunger is an O-ring plunger.

71. The use of the container of claim 69, in which the plunger is a two-position plunger having a first position for use while storing the primary drug container and a second position for use while dispensing a drug from the primary drug container.

72. The use of the container of claim 69, further comprising a hypodermic needle having an internal delivery passage communicating with the lumen and a distal end.

73. The use of the container of claim 72, further comprising a needle shield.

74. The use of the container of claim 73, in which the needle distal end is buried in the needle shield.

75. The use of the container of claim 62, in which said particle count is measured one day after the polypeptide composition is placed in the lumen.
